(19) United States
(12) Patent Application Publication
(10) Pub. No.: US 2006/0142247 A1
(43) Pub. Date: Jun. 29, 2006

(54) Tricyclic heterocycles

(76) Inventors: Guy Georges, Habach (DE); Bernhard Goller, Penzberg (DE); Klaus-Peter Kuenkele, Benediktbeuern (DE); Anja Limberg, Penzberg (DE); Ulrike Reiff, Penzberg (DE); Petra Rueger, Penzberg (DE); Matthias Rueth, Penzberg (DE); Christine Schuell, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/301,993

(22) Filed: Dec. 13, 2005

(30) Foreign Application Priority Data
Dec. 17, 2004 (EP) 04030114.5

(51) Int. Cl. C07F 7/02; A61K 31/695; A61K 31/675; C07D 471/02

(52) U.S. Cl. 514/63; 514/292; 546/82; 514/80; 546/22; 546/14

(57) ABSTRACT

The present invention relates to the compounds of formula I 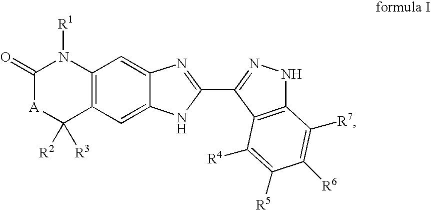 their pharmaceutically acceptable salts or esters, enantiomeric forms, diastereoisomers and racemates, the preparation of the above-mentioned compounds, pharmaceutical compositions containing such compounds and their manufacture, as well as the use of such compounds in the control or prevention of illnesses such as cancer.

### PRIORITY TO RELATED APPLICATIONS

This application claims the benefit of European Application No. 04030114.5, filed Dec. 17, 2004, which is hereby incorporated by reference in its entirety.

### FIELD OF THE INVENTION

The present invention relates to novel tricycles, to a process for their manufacture, pharmaceutical compositions containing them and their manufacture as well as the use of these compounds as pharmaceutically active agents.

### BACKGROUND OF THE INVENTION

Protein kinases regulate many different signaling processes by adding phosphate groups to proteins (Hunter, T., Cell 50 (1987) 823-829); particularly serine/threonine kinases phosphorylate proteins on the alcohol moiety of serine or threonine residues. The serine/threonine kinase family includes members that control cell growth, migration, differentiation, gene expression, muscle contraction, glucose metabolism, cellular protein synthesis, and regulation of the cell cycle.

The Aurora kinases are a family of serine/threonine kinases that are believed to play a key role in the protein phosphorylation events that are essential for the completion of essential mitotic events. The Aurora kinase family is made up of three key members: Aurora A, B and C (also known as Aurora-2, Aurora-1 and Aurora-3 respectively). Aurora-1 and Aurora-2 are described in U.S. Pat. No. 6,207,401 and in related patents and patent applications, e.g. EP 0 868 519 and EP 1 051 500.

For Aurora A there is increasing evidence that it is a novel proto-oncogene. Aurora A gene is amplified and transcript/protein is highly expressed in a majority of human tumor cell lines and primary colorectal, breast and other tumors. It has been shown that Aurora A overexpression leads to genetic instability shown by amplified centrosomes and significant increase in aneuploidy and transforms Rat1 fibroblasts and mouse NIH3T3 cells in vitro. Aurora A-transformed NIH3T3 cells grow as tumors in nude mice (Bischoff, J. R., and Plowman, G. D., Trends Cell Biol. 9 (1999) 454-459; Giet, R., and Prigent, C., J. Cell Sci. 112 (1999) 3591-3601; Nigg, E. A., Nat. Rev. Mol. Cell Biol. 2 (2001) 21-32; Adams, R. R., et al., Trends Cell Biol. 11 (2001) 49-54). Moreover, amplification of Aurora A is associated with aneuploidy and aggressive clinical behavior (Sen, S., et al., J. Natl. Cancer Inst. 94 (2002) 1320-1329) and amplification of its locus correlates with poor prognosis for patients with node-negative breast cancer (Isola, J. J., et al., Am. J. Pathology 147 (1995) 905-911). For these reasons it is proposed that Aurora A overexpression contributes to cancer phenotype by being involved in chromosome segregation and mitotic checkpoint control.

Human tumor cell lines depleted of Aurora A transcripts arrest in mitosis. Accordingly, the specific inhibition of Aurora kinase by selective inhibitors is recognized to stop uncontrolled proliferation, re-establish mitotic checkpoint control and lead to apoptosis of tumor cells. In a xenograft model, an Aurora inhibitor therefore slows tumor growth and induces regression (Harrington, E. A., et al., Nat. Med. 10 (2004) 262-267).

Low molecular weight inhibitors for protein kinases are widely known in the state of the art. For Aurora inhibition such inhibitors are based on for example: quinazoline derivatives as disclosed in the following patent application publications: WO 00/44728; WO 00/47212; WO 01/21594; WO 01/21595; WO 01/21596; WO 01/21597; WO 01/77085; WO 01/55116; WO 95/19169; WO 95/23141; WO 97/42187; and WO 99/06396; pyrazole and triazole derivatives as disclosed in the following patent application publications: WO 02/22601; WO 02/22602; WO 02/22603; WO 02/22604; WO 02/22605; WO 02/22606; WO 02/22607; WO 02/22608; WO 02/50065; WO 02/50066; WO 02/057259; WO 02/059112; WO 02/059111; WO 02/062789; WO 02/066461; and WO 02/068415; pyrimidine derivatives as disclosed in the following patent application publications: WO 03/077921; WO 03/078423; WO 03/078426; WO 03/078427; WO 04/000833; and imidazole, oxazole and thiazole derivatives as disclosed in the following patent application publications: WO 02/96905; and WO 04/005283.

Some tricycles or related compounds are known as inhibitors of erythrocyte aggregation as disclosed in Mertens, A., et al., J. Med. Chem. 30 (1987) 1279-1287; von der Saal, W., et al., J. Med. Chem. 32 (1989) 1481-1491; U.S. Pat. No. 4,666,923; U.S. Pat. No. 4,695,567; U.S. Pat. No. 4,863,945 and U.S. Pat. No. 4,954,498.

WO 03/035065 relates to benzimidazole derivatives as kinase inhibitors, especially as inhibitors against KDR, SYK and ITK tyrosine kinases. WO 01/02369 and WO 01/53268 relate to indazole derivatives as kinase inhibitors, especially as inhibitors against VGEF, LCK, FAK, TEK, CHK-1 and CDKs, with antiproliferative activity.

Many diseases are associated with abnormal cellular responses triggered by protein kinase mediated events. These diseases include autoimmune diseases, inflammatory diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents.

### SUMMARY OF THE INVENTION

The present invention relates to tricyclic heterocycles of the general formula I and all pharmaceutically acceptable salts or esters thereof wherein formula I is: 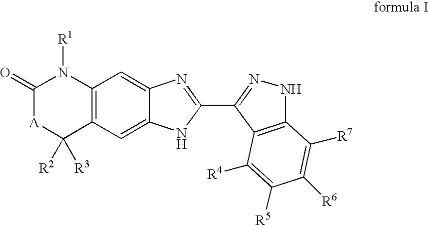
wherein:
(a) R¹ is selected from the group consisting of:
   (1) hydrogen;
   (2) alkyl, which is optionally substituted one or more times with halogen, hydroxy, alkoxy, amino, alkylamino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH—, or phenyl-S(O)₂—NH—;
   (3) alkenyl, which is optionally substituted one or more times with halogen, hydroxy, alkoxy, amino, alkylamino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH—, or phenyl-S(O)₂—NH—;
   (4) alkynyl, which is optionally substituted one or more times with halogen, hydroxy, alkoxy, amino, alkylamino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH—, or phenyl-S(O)₂—NH—;
   (5) arylalkyl, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄)alkyl, halogenated (C₁-C₄)alkoxy or alkylsulfonyl;
   (6) heteroarylalkyl,
      wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl or halogen;
   (7) heterocyclyl-C(O)—(CH₂)ₙ—;
   (8) R⁸—NH—C(O)—(CH₂)ₙ—; and
   (9) R⁹—C(O)—NH—(CH₂)ₙ—;
(b) R⁸ is selected from the group consisting of:
   (1) hydroxyl;
   (2) alkoxy;
   (3) benzyloxy;
   (4) alkyl, which is optionally substituted one to three times with halogen, hydroxy, alkoxy, amino, alkylamino or dialkylamino;
   (5) phenyl-(CH₂)ₘ—, wherein the phenyl is optionally substituted one to three times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
   (1) cycloalkyl, wherein said alkyl is optionally substituted one to three times with halogen, hydroxy, alkoxy, amino, alkylamino or dialkylamino;
   (2) heterocyclyl;
   (3) benzylamino;
   (4) alkyl, wherein said alkyl is optionally substituted one to three times with halogen, hydroxy, alkoxy, amino, alkylamino or dialkylamino;
   (5) phenyl-(CH₂)ₘ—, wherein the phenyl is optionally substituted one to three times with halogen, cyano, nitro, amino, hydroxy, (C₁-C₄)alkyl, (C₁-C₄)alkoxy, halogenated (C₁-C₄)alkyl or halogenated (C₁-C₄)alkoxy; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) n is 1, 2 or 3;
(e) m is 0or 1;
(f) R² and R³ form together with the carbon atom to which they are attached a cycloalkyl ring; or alternatively, R² is hydrogen or alkyl and R³ is hydrogen or alkyl;
(g) R⁴ is hydrogen or halogen;
(h) R⁷ is hydrogen or halogen;
(i) R⁵ is selected from the group consisting of:
   (1) hydrogen;
   (2) halogen;
   (3) cyano;
   (4) nitro;
   (5) amino;
   (6) hydroxyl;
   (7) sulfonic acid;
   (8) carboxylic acid;
   (9) CH₃O—C(O)—;
   (10) H₂N—C(O)—;
   (11) CH₃O—N(CH₃)—C(O)—;
   (12) cycloalkyl-X—, wherein the alkyl is optionally substituted one or more times with halogen;
   (13) heterocyclyl-X—;
   (14) alkyl, which is optionally substituted one or more times with halogen;
   (15) alkyl-X—, wherein the alkyl is optionally substituted one or more times with halogen;
   (16) aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, halogenated (C₁-C₄)alkoxy, or alkylsulfonyl;
   (17) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
   (18) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl; and
   (19) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(j) R⁶ is selected from group consisting of:
   (1) hydrogen;
   (2) halogen;
   (3) cyano;
   (4) nitro;
   (5) amino;
   (6) hydroxy;
   (7) sulfonic acid;
   (8) carboxylic acid;
   (9) CH₃O—C(O)—;
   (10) H₂N—C(O)—;
   (11) CH₃O—N(CH₃)—C(O)—;
   (12) cycloalkyl-X—, wherein the alkyl is optionally substituted one or more times with halogen;
   (13) heterocyclyl-X—;
   (14) alkyl, which is optionally substituted one or more times with halogen;
   (15) alkyl-X—, wherein the alkyl is optionally substituted one or more times with halogen;
   (16) aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, halogenated (C₁-C₄)alkoxy, or alkylsulfonyl;
   (17) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
   (18) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl; and
   (19) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(k) X is —NH—, —N(alkyl)-, —O—, —S(O)₂NH—, —NHS(O)₂—, —NHC(O)—, —N(alkyl)C(O)—, —C(O)—, —OC(O)NH—, —C(O)NH— or —C(O)N(alkyl)-; and
(l) A is a single bond or —CH₂—.

The compounds of the present invention show activity as protein kinase inhibitors and therefore such compounds are useful for preventing or treating diseases associated with abnormal cellular responses triggered by protein kinase mediated events. In particular, the compounds of the present invention show activity as Aurora family kinase inhibitors, especially as Aurora A kinase inhibitors, and may therefore be useful for the treatment of diseases mediated by said kinase. Aurora A inhibition leads to cell cycle arrest in the G2 phase of the cell cycle and exerts an antiproliferative effect in tumor cell lines. This indicates that Aurora A inhibitors may be useful in the treatment of hyperproliferative diseases such as cancer and in particular colorectal, breast, lung, prostate, pancreatic, gastric, bladder, ovarian, melanoma, neuroblastoma, cervical, kidney renal cancers, leukemias, and lymphomas. Aurora A inhibitors may also be useful for the treatment of acute-myelogenous leukemia (AML), acute lymphocytic leukemia (ALL) and gastrointestinal stromal tumor (GIST).

The present invention provides compounds of formula I and their tautomers, pharmaceutically acceptable salts or esters, enantiomeric forms, diastereoisomers and racemates, their use as Aurora kinase inhibitors, the preparation of the above-mentioned compounds, compositions containing them and their manufacture as well as the use of the above-mentioned compounds in the control or prevention of illnesses, especially the illnesses and disorders mentioned above, or in the manufacture of corresponding pharmaceutical compositions.

### DETAILED DESCRIPTION OF THE INVENTION

The term “alkyl” as used herein means a saturated, straight-chain or branched-chain hydrocarbon containing from 1 to 6 carbon atoms, preferably 1 to 4 carbon atoms, such as methyl, ethyl, n-propyl, isopropyl, n-butyl, 2-butyl, and t-butyl.

The term “alkenyl” as used herein means an unsaturated straight-chain or branched aliphatic hydrocarbon group containing one double bond and having 2 to 6 carbon atoms, preferably 2 to 4 carbon atoms. Examples of such “alkenyl” groups are vinyl (ethenyl), allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, preferably allyl and 3-butenyl.

The term “alkynyl” as used herein means an unsaturated straight-chain or branched aliphatic hydrocarbon group containing one triple bond and having 2 to 6, preferably 2 to 4 carbon atoms. Examples of such “alkynyl” groups are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “alkoxy” as used herein means an alkyl-O— group wherein the alkyl is defined as above.

The term “alkylamino” as used herein means an alkyl-NH— group wherein the alkyl is defined as above.

The term “dialkylamino” as used herein means an (alkyl)₂N— group wherein the alkyl is defined as above.

The term “dialkylphosphinoyl” as used herein means a (alkyl)₂P(═O)— group wherein the alkyl is defined as above.

The term “alkylsulfanyl” as used herein means an alkyl-S— group wherein the alkyl is defined as above.

The term “alkylsulfinyl” as used herein means an alkyl-S(O)— group wherein the alkyl is defined as above.

The term “alkylsulfonyl” as used herein means an alkyl-S(O)₂— group wherein the alkyl is defined as above.

The term “alkoxyalkoxy” as used herein means an alkoxy group as defined above which attached to the alkyl of a second alkoxy group. Examples of an “alkoxyalkoxy” include 2-methoxy-ethoxy, 2-ethoxy-ethoxy, 1-ethoxy-ethoxy, 3-methoxy-propoxy, 2-methoxy-propoxy, methoxy-methoxy and the like.

If the alkyl group is “optionally substituted one or more times with halogen”, it is preferably substituted by fluorine. Examples are difluoromethyl, trifluoromethyl, 2,2,2-trifluoroethyl, perfluorethyl, difluoromethoxy, trifluoromethoxy, 2,2,2-trifluoroethoxy, perfluoroethoxy and the like, especially trifluoromethyl and trifluoromethoxy.

The term “halogenated alkyl” as used herein means an alkyl group as defined above which is substituted one or more times with halogen, preferably by fluorine or chlorine, especially fluorine. Examples are difluoromethyl, trifluoromethyl, 2,2,2-trifluoroethyl, perfluorethyl, and the like, especially trifluoromethyl.

The term “halogenated alkoxy” as used herein means an alkoxy group as defined above which is substituted one or more times with halogen, preferably by fluorine or chlorine, especially fluorine. Examples are difluoromethoxy, trifluoromethoxy, 2,2,2-trifluoroethoxy, perfluoroethoxy and the like, especially trifluoromethoxy.

The term “halogen” as used in the definitions of R¹, R⁵ and R⁶ means fluorine, chlorine, bromine or iodine, preferably fluorine, chlorine or bromine and especially fluorine or chlorine.

The term “halogen” as used in the definitions of R⁴ and R⁷ means fluorine, chlorine or bromine, preferably fluorine or chlorine and especially fluorine.

The term “cycloalkyl” means a monocyclic saturated hydrocarbon ring with 3 to 7 ring atoms, preferably 3 to 6 ring atoms. Examples of such saturated carbocyclic groups are cyclopropyl, cyclobutyl, cyclopentyl, cyclohexyl and cycloheptyl, preferably cyclopropyl. The cycloalkyl ring which is formed by R² and R³ together with the carbon atom to which they are attached is preferably a cyclopentyl or cyclohexyl ring, especially a cyclopentyl ring.

The term “heterocyclyl” as used herein means a saturated, monocyclic hydrocarbon ring with 5 to 6 ring atoms which contains up to 3, preferably 1 or 2 heteroatoms selected independently from the group consisting of N, O and S with the remaining ring atoms being carbon atoms. Such a saturated heterocyclic group can be optionally substituted one to three, preferably one or two times with (a) alkyl, as defined as above, preferably methyl; (b) —C(O)-alkyl, preferably acetyl, (c) oxo; or (d) —S(O)₂-alkyl. Preferably the heterocyclic group is optionally substituted by alkyl. Examples of such saturated heterocyclic groups are pyrrolidinyl, morpholinyl, piperazinyl, N-methyl-piperazinyl, piperidyl, N-acetyl-piperazinyl, N-methanesulfonyl-piperazinyl, N-isopropyl-piperazinyl, thiazolidinyl, thiomorpholinyl, 1,1-dioxo-1λ⁶-thiomorpholin-4-yl (or 1,1-dioxido-thiomorpholin-4-yl), 1-oxo-1λ⁴-thiomorpholin-4-yl (or 1-oxido-thiomorpholin-4-yl) and the like, preferably pyrrolidinyl, morpholinyl, piperazinyl, N-methyl-piperazinyl, piperidyl and more preferably morpholinyl.

The term “aryl” as used herein means a mono- or bicyclic aromatic ring with 6 to 10 ring carbon atoms. Examples of such aryl groups are phenyl and naphthyl, preferably phenyl.

The term “heteroaryl” as used herein means a mono- or bicyclic aromatic ring with 5 to 10, preferably 5 to 6, ring atoms, which contains up to 4, preferably up to 3, more preferably 1 or 2 heteroatoms selected independently from the group consisting of N, O and S with the remaining ring atoms being carbon atoms. Examples of such heteroaryl groups are pyridyl, thienyl, benzimidazolyl, pyrimidyl, thiazolyl, tetrazolyl, quinolyl, pyridazinyl, pyrazinyl, oxazolyl, quinazolinyl, indolyl, benzothiophenyl, benzofuranyl and the like, preferably pyridyl, thienyl, benzimidazolyl, pyrimidyl, thiazolyl, tetrazolyl, quinolyl or pyridazinyl, and especially pyridyl.

In one preferred embodiment of the invention the heteroaryl in the heteroarylalkyl group as defined in R¹ is selected from the group consisting of pyridyl, thiazolyl, tetrazolyl, thienyl, pyrimidyl, and pyridazinyl; and is preferably pyridyl, thiazolyl or tetrazolyl.

In one preferred embodiment of the invention the heteroaryl in the definition of R⁸ and R⁹ is selected from the group consisting of pyridyl and thienyl; and is preferably pyridyl.

In one preferred embodiment of the invention the heteroaryl in the heteroarylalkyl group as defined in R⁵ and R⁶ is selected from the group consisting of pyridyl and thienyl, and is preferably pyridyl.

In one preferred embodiment of the invention the heteroaryl in the definition of R⁵ and R⁶ is selected from the group consisting of pyridyl and thienyl, and is preferably pyridyl.

The term “arylalkyl” as used herein means a (C₁-C₄)alkyl group as defined above in which one of the hydrogen atoms is replaced by an aryl group. Examples of arylalkyl groups are benzyl, 2-phenylethyl, 3-phenylpropyl, 4-chlorobenzyl, 4-methoxybenzyl and the like, preferably benzyl.

The term “heteroarylalkyl” as used herein means a (C₁-C₄)alkyl group as defined above, in which one of the hydrogen atoms is replaced by a heteroaryl group. Examples of heteroarylalkyl groups are pyridylmethyl, thienylmethyl and the like.

The term “optionally substituted one or more times” preferably refers to a substituent or group that is optionally substituted one to six times, and more preferably one to three times. If the aryl (or aryl part of the arylalkyl group) in the definitions of R¹, R⁵ or R⁶ is substituted one or several times it is substituted preferably one to three, and more preferably one or two times. If the heteroaryl (or heteroaryl part of heteroarylalkyl group) in the definitions of R¹, R⁵ or R⁶ is substituted one or several times it is substituted preferably one or two, and more preferably one time.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See e.g. Stahl, P. H., and Wermuth, G., (editors), Handbook of Pharmaceutical Salts, Verlag Helvetica Chimica Acta (VHCA), Zürich, (2002) or Bastin, R. J., et al., Organic Proc. Res. Dev. 4 (2000) 427-435.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases which are commercially available.

The compounds of formula I can exist in different tautomeric forms and in variable mixtures thereof. All tautomeric forms of the compounds of formula I and mixtures thereof are an objective of the invention. For example, the imidazole part of the tricyclic ring system of formula I can exist in two tautomeric forms as shown here below:

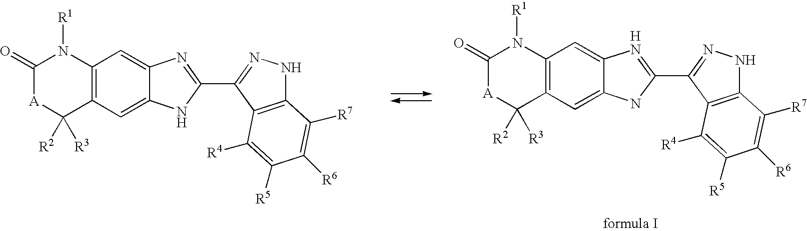

As used herein, in relation to mass spectrometry (MS) the term “API+” refers to positive atmospheric pressure ionization mode, the term “API−” refers to negative atmospheric pressure ionization mode, the term “ESI+” refers to positive electrospray ionization mode and the term “ESI−” refers to negative electrospray ionization mode.

An embodiment of the invention are the compounds of formula I and all pharmaceutically acceptable salts or esters thereof, wherein:
(a) R¹ is selected from the group consisting of:
   (1) hydrogen;
   (2) alkly, which is optionally substituted one or more times with halogen, hydroxy, alkoxy, amino, alkylamino, dialkylamino, cycloalkyl, heterocyclyl, or dialkylphosphinoyl;
   (3) alkenyl, which is optionally substituted one or more times with halogen, hydroxy, alkoxy, amino, alkylamino, dialkylamino, cycloalkyl, heterocyclyl, or dialkylphosphinoyl;
   (4) alkynyl, which is optionally substituted one or more times with halogen, hydroxy, alkoxy, amino, alkylamino, dialkylamino, cycloalkyl, heterocyclyl, or dialkylphosphinoyl;
   (5) arylalkyl, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
   (6) heteroarylalkyl, wherein the heteroaryl is optionally substituted one or more times with alkyl or halogen;
R² and R³ form together with the carbon atom to which they are attached a cycloalkyl ring; or alternatively, R² is hydrogen or alkyl and R³ is hydrogen or alkyl;
(c) R⁴ is hydrogen or halogen;
(d) R⁷ is hydrogen or halogen;
(e) R⁵ is selected from the group consisting of:
   (1) hydrogen;
   (2) halogen;
   (3) cyano;
   (4) nitro;
   (5) amino;
   (6) hydroxyl;
   (7) sulfonic acid;
   (8) carboxylic acid;
   (9) alkyl, which is optionally substituted one or more times with halogen;
   (10) alkyl-X—, wherein the alkyl is optionally substituted one or more times with halogen;
   (11) aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
   (12) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
   (13) heteroaryl-X—, wherein the heteroaryl is optionally substituted one or more times with alkyl;
(f) R⁶ is selected from group consisting of:
   (1) hydrogen;
   (2) halogen;
   (3) cyano;
   (4) nitro;
   (5) amino;
   (6) hydroxy;
   (7) sulfonic acid;
   (8) carboxylic acid;
   (9) alkyl, which is optionally substituted one or more times with halogen;
   (10) alkyl-X—, wherein the alkyl is optionally substituted one or more times with halogen;
   (11) aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
   (12) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
   (13) heteroaryl-X—, wherein the heteroaryl is optionally substituted one or more times with alkyl;
(g) X is —NH—, —N(alkyl)-, —O—, —S(O)₂NH—, —NHS(O)₂—, —NHC(O)—, —N(alkyl)C(O)—, —C(O)NH— or —C(O)N(alkyl)-; and
(h) A is a single bond or —CH₂—.

Another embodiment of the invention are the compounds of formula I, wherein A is a single bond.

Another embodiment of the invention are the compounds of formula I, wherein A is —CH₂—.

Another embodiment of the invention are the compounds of formula I, wherein: R¹ is selected from the group consisting of:
(a) hydrogen;
(b) alkyl, which is optionally substituted one or more times with halogen, hydroxy, alkoxy, amino, alkylamino dialkylamino, cycloalkyl, heterocyclyl or dialkylphosphinoyl;
(c) alkenyl, which is optionally substituted one or more times with halogen, hydroxy, alkoxy, amino, alkylamino dialkylamino, cycloalkyl, heterocyclyl or dialkylphosphinoyl; and
(d) alkynyl, which is optionally substituted one or more times with halogen, hydroxy, alkoxy, amino, alkylamino dialkylamino, cycloalkyl, heterocyclyl or dialkylphosphinoyl.

Another embodiment of the invention are the compounds of formula I, wherein A is a single bond and R¹ is selected from the group consisting of:
(a) hydrogen;
(b) alkyl, which is optionally substituted one or more times with halogen, hydroxy, alkoxy, amino, alkylamino dialkylamino, cycloalkyl, heterocyclyl or dialkylphosphinoyl;
(c) alkenyl, which is optionally substituted one or more times with halogen, hydroxy, alkoxy, amino, alkylamino dialkylamino, cycloalkyl, heterocyclyl or dialkylphosphinoyl; and
(d) alkynyl, which is optionally substituted one or more times with halogen, hydroxy, alkoxy, amino, alkylamino dialkylamino, cycloalkyl, heterocyclyl or dialkylphosphinoyl.

Another embodiment of the invention are the compounds of formula I, wherein R¹ is selected from the group consisting of:
(a) hydrogen;
(b) alkyl; and
(c) alkenyl.

Another embodiment of the invention are the compounds of formula I, wherein A is a single bond and R¹ is selected from the group consisting of:
(a) hydrogen;
(b) alkyl; and
(c) alkenyl.

Such compounds may, for example, be selected from the group consisting of:
(a) 5-Ethyl-2-(1H-indazol-3-yl)-7,7-dimethyl-5,7-dihydro-3H-imidazo[4,5-f]indol-6-one;
2-(1H-Indazol-3-yl)-5,7,7-trimethyl-5,7-dihydro-3H-imidazo[4,5-f]indol-6-one;
2-(1H-Indazol-3-yl)-7,7-dimethyl-5-propyl-5,7-dihydro-3H-imidazo[4,5-f]indol-6-one;
2-(1H-Indazol-3-yl)-5-isopropyl-7,7-dimethyl-5,7-dihydro-3H-imidazo[4,5-f]indol-6-one;
2-(1H-Indazol-3-yl)-7,7-dimethyl-5,7-dihydro-3H-imidazo[4,5-]indol-6-one;
2-(1H-Indazol-3-yl)-spiro[7,7-cyclopentan-5,7-dihydro-3H-imidazo[4,5-f]indol-6]-one; and
5-Allyl-2-(1H-indazol-3-yl)-7,7-dimethyl-5,7-dihydro-3H-imidazo[4,5-f]indol-6-one.

Another embodiment of the invention are the compounds of formula I, wherein A is —CH₂— and R¹ is selected from the group consisting of:
(a) hydrogen;
(b) alkyl; and
(c) alkenyl.

Such a compound is for example:
2-(1H-Indazol-3-yl)-8,8-dimethyl-1,5,7,8-tetrahydro-imidazo[4,5-g]quinolin-6-one.

Another embodiment of the invention are the compounds of formula I, wherein A is a single bond and R¹ is alkyl, wherein said alkyl is substituted one or more times with halogen, hydroxy, alkoxy, amino, alkylamino dialkylamino, cycloalkyl, heterocyclyl or dialkylphosphinoyl.

Such compounds, for example, may be selected from the group consisting of:
2-(1H-Indazol-3-yl)-7,7-dimethyl-5-(3-morpholin-4-yl-propyl)-5,7-dihydro-3H-imidazo[4,5-f]indol-6-one; and
5-Cyclopropylmethyl-2-(1H-indazol-3-yl)-7,7-dimethyl-5,7-dihydro-3H-imidazo[4,5-f]indol-6-one.

Another embodiment of the invention are the compounds of formula I, wherein A is a single bond and R¹ is arylalkyl, wherein the aryl is optionally substituted one or more times with halogen, cyano, nitro, amino, hydroxy, (C₁-C₄)alkyl, (C₁-C₄)alkoxy, halogenated (C₁-C₄)alkyl or halogenated (C₁-C₄)alkoxy.

Another embodiment of the invention are the compounds of formula I, wherein A is a single bond and R¹ is heteroarylalkyl, wherein the heteroaryl is optionally substituted one or more times with alkyl or halogen.

An embodiment of the invention are the compounds of formula I, wherein A is a single bond and R⁴, R⁵, R⁶ and R⁷ represent hydrogen.

Another embodiment of the invention are the compounds of formula I, wherein:
(a) R¹ is selected from the group consisting of:
   (1) hydrogen;
   (2) alkyl, which is optionally substituted one or more times with halogen, hydroxy, alkoxy, amino, alkylamino dialkylamino, cycloalkyl, heterocyclyl or dialkylphosphinoyl;
   (3) alkenyl, which is optionally substituted one or more times with halogen, hydroxy, alkoxy, amino, alkylamino dialkylamino, cycloalkyl, heterocyclyl or dialkylphosphinoyl; and
   (4) alkynyl, which is optionally substituted one or more times with halogen, hydroxy, alkoxy, amino, alkylamino dialkylamino, cycloalkyl, heterocyclyl or dialkylphosphinoyl;
(b) R⁴, R⁵, R⁶ and R⁷ represent hydrogen; and
(c) A is a single bond.

Another embodiment of the invention are the compounds of formula I, wherein:
(a) R¹ is alkyl;
(b) R⁴, R⁵, R⁶ and R⁷ represent hydrogen; and
(c) A is a single bond.

Another embodiment of the invention are the compounds of formula I, wherein:
(a) R¹ is hydrogen, alkyl, or alkenyl;
(b) R⁴, R⁵, R⁶ and R⁷ represent hydrogen; and
(c) A is a single bond.

Another embodiment of the invention are the compounds of formula I, wherein:
(a) R¹ is alkyl, wherein said alkyl is substituted one or more times with halogen, hydroxy, alkoxy, amino, alkylamino dialkylamino, cycloalkyl, heterocyclyl or dialkylphosphinoyl;
(b) R⁴, R⁵, R⁶ and R⁷ represent hydrogen; and
(c) A is a single bond.

Another embodiment of the invention are the compounds of formula I, wherein:
(a) R¹ is arylalkyl, wherein the aryl is optionally substituted one or more times with halogen, cyano, nitro, amino, hydroxy, (C₁-C₄)alkyl, (C₁-C₄)alkoxy, halogenated (C₁-C₄)alkyl or halogenated (C₁-C₄)alkoxy;
(b) R⁴, R⁵, R⁶ and R⁷ represent hydrogen; and
(c) A is a single bond.

Another embodiment of the invention are the compounds of formula I, wherein:
(a) R¹ is heteroarylalkyl, wherein the heteroaryl is optionally substituted one or more times with alkyl or halogen;
(b) R⁴, R⁵, R⁶ and R⁷ represent hydrogen; and
(c) A is a single bond.

Another embodiment of the invention are the compounds of formula I, wherein X is —NH—, —N(alkyl)- or —O—.

Another embodiment of the invention are the compounds of formula I, wherein X is —NH—, —N(alkyl)- or —O—; and A is a single bond.

Another embodiment of the invention are the compounds of formula I, wherein:
(a) R¹ is hydrogen; alkyl or alkenyl; and
(b) X is —NH—, —N(alkyl)- or —O—.

Another embodiment of the invention are the compounds of formula I, wherein:
(a) R¹ is hydrogen, alkyl or alkenyl;
(b) X is —NH—, —N(alkyl)- or —O—; and
(c) A is a single bond.

Another embodiment of the invention are the compounds of formula I, wherein X is —S(O)₂NH— or —NHS(O)₂—.

Another embodiment of the invention are the compounds of formula I, wherein X is —S(O)₂NH— or —NHS(O)₂—; and A is a single bond.

Another embodiment of the invention are the compounds of formula I, wherein:
(a) R¹ is hydrogen, alkyl or alkenyl;
(b) X is —S(O)₂NH— or —NHS(O)₂—; and
(c) A is a single bond.

Another embodiment of the invention are the compounds of formula I, wherein X is —NHC(O)—, —N(alkyl)C(O)—, —C(O)NH— or —C(O)N(alkyl)-.

Another embodiment of the invention are the compounds of formula I, wherein:
(a) X is —NHC(O)—, —N(alkyl)C(O)—, —C(O)NH— or —C(O)N(alkyl)-; and
(b) A is a single bond.

Another embodiment of the invention are the compounds of formula I, wherein:
(a) R¹ is hydrogen, alkyl or alkenyl;
(b) X is —NHC(O)—, —N(alkyl)C(O)—, —C(O)NH— or —C(O)N(alkyl)-; and
(c) A is a single bond.

Another embodiment of the invention are the compounds of formula I, wherein R⁵ is hydrogen.

Another embodiment of the invention are the compounds of formula I, wherein R⁵ is hydrogen; and A is a single bond.

Another embodiment of the invention are the compounds of formula I, wherein:
(a) R¹ is hydrogen, alkyl or alkenyl;
(b) R⁵ is hydrogen; and
(c) A is a single bond.

Another embodiment of the invention are the compounds of formula I, wherein R⁶ is hydrogen.

Another embodiment of the invention are the compounds of formula I, wherein R⁶is hydrogen; and A is a single bond.

Another embodiment of the invention are the compounds of formula I, wherein:
(a) R¹ is hydrogen, alkyl or alkenyl; and
(b) R⁶ is hydrogen.

Another embodiment of the invention are the compounds of formula I, wherein:
(a) R¹ is hydrogen, alkyl or alkenyl;
(b) R⁶ is hydrogen; and
(c) A is a single bond.

Another embodiment of the invention are the compounds of formula I, wherein:
(a) R⁵ is selected from the group consisting of:
   (1) halogen;
   (2) cyano;
   (3) nitro;
   (4) amino;
   (5) hydroxy;
   (6) sulfonic acid;
   (7) carboxylic acid; and
   (8) alkyl, wherein the alkyl group is optionally substituted one or more times with halogen;
(b) R⁶ is hydrogen; and
(c) A is a single bond.

Another embodiment of the invention are the compounds of formula I, wherein:
(a) R⁵ is alkyl-X—, wherein the alkyl group is optionally substituted one or more times with halogen;
(b) R⁶ is hydrogen; and
(c) A is a single bond.

Another embodiment of the invention are the compounds of formula I, wherein:
(a) R⁵ is aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
(b) R⁶ is hydrogen; and
(c) A is a single bond.

Another embodiment of the invention are the compounds of formula I, wherein:
(a) R⁵ is selected from the group consisting of:
   (1) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
   (2) heteroaryl-X—, wherein the heteroaryl is optionally substituted one or more times with alkyl;
(b) R⁶ is hydrogen; and
(c) A is a single bond.

Another embodiment of the invention are the compounds of formula I, wherein:
(a) R⁵ is hydrogen;
(b) R⁶ is selected from the group consisting of:
   (1) halogen;
   (2) cyano;
   (3) nitro;
   (4) amino;
   (5) hydroxy;
   (6) sulfonic acid;
   (7) carboxylic acid; and
   (8) alkyl, wherein the alkyl group is optionally substituted one or more times with halogen; and
(c) A is a single bond.

Another embodiment of the invention are the compounds of formula I, wherein:
(a) R⁵ is hydrogen;
(b) R⁶ is alkyl-X—, wherein the alkyl group is optionally substituted one or more times with halogen; and
(c) A is a single bond.

Another embodiment of the invention are the compounds of formula I, wherein:
(a) R⁵is hydrogen;
(b) R⁶ is aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl or halogenated (C₁-C₄)alkoxy; and
(c) A is a single bond.

Another embodiment of the invention are the compounds of formula I, wherein:
(a) R⁵ is hydrogen;
(b) R⁶ is selected from the group consisting of:
   (1) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
   (2) heteroaryl-X—, wherein the heteroaryl is optionally substituted one or more times with alkyl; and
(c) A is a single bond.

Another embodiment of the invention are the compounds of formula I, wherein:
(a) R¹ is selected from the group consisting of:
   (1) hydrogen;
   (2) alkyl, wherein said alkyl is optionally substituted one or more times with hydroxy, alkoxy, amino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH— or phenyl-S(O)₂—NH—;
   (3) alkenyl;
   (4) arylalkyl, wherein the aryl is a mono- or bicyclic aromatic ring with 6 to 10 ring carbon atoms and wherein the aryl is optionally substituted one or more times with alkylsulfonyl;
   (5) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
   (6) heterocyclyl-C(O)—(CH₂)ₙ—;
   (7) R⁸—NH—C(O)—(CH₂)ₙ—; and
   (8) R⁹—C(O)—NH—(CH₂)ₙ—;
(b) R⁸ is selected from the group consisting of:
   (1) hydroxyl;
   (2) alkoxy;
   (3) benzyloxy;
   (4) alkyl, wherein said alkyl is optionally substituted one to three times with hydroxy or dialkylamino;
   (5) phenyl-(CH₂)ₘ—, wherein the phenyl is optionally substituted one to three times with halogen or (C₁-C₄)alkoxy; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
   (1) cycloalkyl;
   (2) heterocyclyl;
   (3) benzylamino;
   (4) alkyl;
   (5) phenyl-(CH₂)ₘ—; and
   (6) heteroaryl-(CH₂)ₘ—; wherein the heteroaryl is a mono- or bicyclic aromatic ring with 5 to 10 ring atoms, which contains up to 4 heteroatoms selected independently from the group consisting of N, O and S, with the remaining ring atoms being carbon atoms;
(d) n is 1, 2 or 3;
(e) m is 0 or 1;
(f) R⁴ and R⁷ each represent hydrogen;
(g) R⁵ is selected from the group consisting of:
   (1) hydrogen;
   (2) halogen;
   (3) cyano;
   (4) nitro;
   (5) amino;
   (6) carboxylic acid;
   (7) CH₃O—C(O)—;
   (8) H₂N—C(O)—;
   (9) CH₃O—N(CH₃)—C(O)—;
   (10) cycloalkyl-X—;
   (11) heterocyclyl-X—;
   (12) alkyl-X—, wherein the alkyl group is optionally substituted one or more times with halogen;
   (13) aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
   (14) arylalkyl-X—, wherein the aryl is a mono- or bicyclic aromatic ring with 6 to 10 ring carbon atoms and wherein the aryl is optionally substituted one or more times with halogen, (C₁-C₄)alkyl, (C₁-C₄)alkoxy or halogenated (C₁-C₄)alkoxy;
   (15) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms; and
   (16) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(h) R⁶ is selected from the group consisting of:
   (1) hydrogen;
   (2) halogen;
   (3) carboxylic acid;
   (4) H₂N—C(O)—;
   (5) alkyl-X—;
   (6) aryl-X—, wherein the aryl is a mono- or bicyclic aromatic ring with 6 to 10 ring carbon atoms; and
   (7) arylalkyl-X—, wherein the aryl is a mono- or bicyclic aromatic ring with 6 to 10 ring carbon atoms; and
(i) X is —NH—, —O—, —S(O)₂NH—, —NHC(O)—, —C(O)—, —OC(O)NH— or —C(O)NH—.

A further embodiment of the invention is the embodiment above, wherein A is a single bond.

Another embodiment of the invention are the compounds of formula I, wherein:
(a) R¹ is selected from the group consisting of:
   (1) hydrogen;
   (2) alkyl;
   (3) alkenyl, wherein said alkyl is optionally substituted one or more times with hydroxy, alkoxy, amino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH— or phenyl-S(O)₂—NH—;
   (4) arylalkyl, wherein the aryl is a mono- or bicyclic aromatic ring with 6 to 10 ring carbon atoms and wherein the aryl is optionally substituted one or more times with alkylsulfonyl;
   (5) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
   (6) heterocyclyl-C(O)—(CH₂)ₙ—;
   (7) R⁸—NH—C(O)—(CH₂)ₙ—; and
   (8) R⁹—C(O)—NH—(CH₂)ₙ—;
(b) R⁸ is selected from the group consisting of:
   (1) hydroxy;
   (2) alkoxy;
   (3) benzyloxy;
   (4) alkyl, wherein said alkyl is optionally substituted one to three times with hydroxy or dialkylamino;
   (5) phenyl-(CH₂)ₘ—, wherein the phenyl is optionally substituted one to three times with halogen or (C₁-C₄)alkoxy; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
   (1) cycloalkyl;
   (2) heterocyclyl;
   (3) benzylamino;
   (4) alkyl;
   (5) phenyl-(CH₂)ₘ—; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) n is 1, 2 or 3;
(e) m is 0 or 1;
(f) R⁴ and R⁷ each represent hydrogen;
(g) R⁵ is selected from the group consisting of:
   (1) hydrogen;
   (2) halogen;
   (3) cyano;
   (4) nitro;
   (5) amino;
   (6) carboxylic acid;
   (7) CH₃O—C(O)—;
   (8) H₂N—C(O)—;
   (9) CH₃O—N(CH₃)—C(O)—;
   (10) cycloalkyl-X—;
   (11) heterocyclyl-X—;
   (12) alkyl-X—, wherein the alkyl group is optionally substituted one or more times with halogen;
   (13) aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
   (14) arylalkyl-X—, wherein the aryl is a mono- or bicyclic aromatic ring with 6 to 10 ring carbon atoms and wherein the aryl is optionally substituted one or more times with halogen, (C₁-C₄)alkyl, (C₁-C₄)alkoxy or halogenated (C₁-C₄)alkoxy;
   (15) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms; and
   (15) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(h) R⁶ is selected from the group consisting of:
   (1) hydrogen;
   (2) halogen;
   (3) carboxylic acid;
   (4) H₂N—C(O)—;
   (5) alkyl-X—;
   (6) aryl-X—, wherein the aryl is a mono- or bicyclic aromatic ring with 6 to 10 ring carbon atoms; and
   (7) arylalkyl-X—, wherein the aryl is a mono- or bicyclic aromatic ring with 6 to 10 ring carbon atoms;
(i) X is —NH—, —O—, —S(O)₂NH—, —NHC(O)—, —C(O)—, —OC(O)NH— or —C(O)NH—; and
(j) A is a single bond.

Another embodiment of the invention are the compounds of formula I, wherein:
(a) R¹ is selected from the group consisting of:
   (1) hydrogen;
   (2) alkyl; wherein said alkyl is optionally substituted one or more times with hydroxy, alkoxy, amino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH— or phenyl-S(O)₂—NH—;
   (3) alkenyl;
   (4) arylalkyl, wherein the aryl is a mono- or bicyclic aromatic ring with 6 to 10 ring carbon atoms and wherein the aryl is optionally substituted one or more times with alkylsulfonyl;
   (5) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
   (6) heterocyclyl-C(O)—(CH₂)ₙ;
   (7) R⁸—NH—C(O)—(CH₂)ₙ—; and
   (8) R⁹—C(O)—NH—(CH₂)ₙ—;
(b) R⁸ is selected from the group consisting of:
   (1) hydroxyl;
   (2) alkoxy;
   (3) benzyloxy;
   (4) alkyl, wherein said alkyl is optionally substituted one to three times with hydroxy or dialkylamino;
   (5) phenyl-(CH₂)ₘ—, wherein the phenyl is optionally substituted one to three times with halogen or (C₁-C₄)alkoxy; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
   (1) cycloalkyl;
   (2) heterocyclyl;
   (3) benzylamino;
   (4) alkyl;
   (5) phenyl-(CH₂)ₘ—; and
   (6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) n is 1, 2 or 3;
(e) m is 0 or 1;
(f) R⁴ and R⁷ each represent hydrogen;
(g) R⁵ is selected from the group consisting of:
   (1) hydrogen;
   (2) halogen;
   (3) cyano;
   (4) nitro;
   (5) amino;
   (6) carboxylic acid;
   (7) CH₃O—C(O)—;
   (8) H₂N—C(O)—;
   (9) CH₃O—N(CH₃)—C(O)—;
   (10) cycloalkyl-X—;
   (11) heterocyclyl-X—;
   (12) alkyl-X—, wherein the alkyl group is optionally substituted one or more times with halogen;
   (13) aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
   (14) arylalkyl-X—, wherein the aryl is a mono- or bicyclic aromatic ring with 6 to 10 ring carbon atoms and wherein the aryl is optionally substituted one or more times with halogen, (C₁-C₄)alkyl, (C₁-C₄)alkoxy or halogenated (C₁-C₄)alkoxy;
   (15) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms; and
   (16) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(h) R⁶ is selected from the group consisting of:
   (1) hydrogen;
   (2) halogen;
   (3) carboxylic acid;
   (4) H₂N—C(O)—;
   (5) alkyl-X—;
   (6) aryl-X—, wherein the aryl is a mono- or bicyclic aromatic ring with 6 to 10 ring carbon atoms; and
   (7) arylalkyl-X—, wherein the aryl is a mono- or bicyclic aromatic ring with 6 to 10 ring carbon atoms;
(i) X is —NH—, —O—, —S(O)₂NH—, —NHC(O)—, —C(O)—, —OC(O)NH— or —C(O)NH—; and
(j) A is a single bond.

Another embodiment of the invention are the compounds of formula I, wherein:
(a) R¹ is hydrogen;
(b) R⁴, R⁵, R⁶ and R⁷ each represent hydrogen; and
(c) A is a single bond.

Such compounds, for example, may be selected from the group consisting of:
2-(1H-Indazol-3-yl)-7,7-dimethyl-5,7-dihydro-3H-imidazo[4,5-f]indol-6-one;
2-(1H-Indazol-3-yl)-spiro[7,7-cyclopentan-5,7-dihydro-3H-imidazo[4,5-f]indol-6]-one or according to the actual IUPAC-nomenclature: 2-(1H-Indazol-3-yl)-spiro-5,7-dihydro[cyclopentane-1′,7-imidazo[4,5-f]indol]-6(3H)-one;
2-(1H-Indazol-3-yl)-7-methyl-5,7-dihydro-3H-imidazo[4,5-f]indol-6-one; and
7-Ethyl-2-(1H-indazol-3-yl) -5,7-dihydro-3H-imidazo[4,5-f]indol-6-one.

Another embodiment of the invention are the compounds of formula I, wherein:
(a) R¹ is alkyl or alkenyl;
(b) R⁴, R⁵, R⁶ and R⁷ each represent hydrogen; and
(c) A is a single bond.

Such compounds, for example, may be selected from the group consisting of:
5-Allyl-2-(1H-indazol-3-yl)-7,7-dimethyl-5,7-dihydro-3H-imidazo[4,5-f]indol-6-one;
5-Ethyl-2-(1H-indazol-3-yl)-7,7-dimethyl-5,7-dihydro-3H-imidazo[4,5-f]indol-6-one;
2-(1H-Indazol-3-yl)-5,7,7-trimethyl-5,7-dihydro-3H-imidazo[4,5-f]indol-6-one;
2-(1H-Indazol-3-yl)-7,7-dimethyl-5-propyl-5,7-dihydro-3H-imidazo[4,5-f]indol-6-one;
2-(1H-Indazol-3-yl)-5-isopropyl-7,7-dimethyl-5,7-dihydro-3H-imidazo[4,5-f]indol-6-one;
5,7,7-Triethyl-2-(1H-indazol-3-yl) -5,7-dihydro-3H-imidazo[4,5-f]indol-6-one; and
5-But-3-enyl-2-(1H-indazol-3-yl)-7,7-dimethyl-5,7-dihydro-3H-imidazo[4,5-f]indol-6-one.

Another embodiment of the invention are the compounds of formula I, wherein:
(a) R¹ is alkyl, wherein said alkyl is substituted one to three times with hydroxy, alkoxy, amino, dialkylamino, dialkylphosphinoyl, alkoxyalkoxy, cyano, cycloalkyl, heterocyclyl, alkylsulfanyl, alkylsulfinyl or alkylsulfonyl;
(b) R⁴, R⁵, R⁶ and R⁷ each represent hydrogen; and
(c) A is a single bond.

Such compounds, for example, may be selected from the group consisting of:
5-Cyclopropylmethyl-2-(1H-indazol-3-yl)-7,7-dimethyl-5,7-dihydro-3H-imidazo[4,5-f]indol-6-one;
2-(1H-Indazol-3-yl)-7,7-dimethyl-5-(3-morpholin-4-yl-propyl)-5,7-dihydro-3H-imidazo[4,5-f]indol-6-one;
2-(1H-Indazol-3-yl)-5-[2-(2-methoxy-ethoxy)-ethyl]-7,7-dimethyl-5,7-dihydro-3H-imidazo[4,5-f]indol-6-one;
2-(1H-Indazol-3-yl)-5-(2-methoxy-ethyl)-7,7-dimethyl-5,7-dihydro-3H-imidazo[4,5-f]indol-6-one;
2-(1H-Indazol-3-yl)-7,7-dimethyl-5-(3-piperidin-1-yl-propyl)-5,7-dihydro-3H-imidazo[4,5-f]indol-6-one;
5-(2-Diisopropylamino-ethyl)-2-(1H-indazol-3-yl)-7,7-dimethyl-5,7-dihydro-3H-imidazo[4,5-f]indol-6-one;
5-(3-Dimethylamino-propyl)-2-(1H-indazol-3-yl)-7,7-dimethyl-5,7-dihydro-3H-imidazo[4,5-f]indol-6-one;
5-(2-Diethylamino-ethyl)-2-(1H-indazol-3-yl)-7,7-dimethyl-5,7-dihydro-3H-imidazo[4,5-f]indol-6-one;
[2-(1H-Indazol-3-yl)-7,7-dimethyl-6-oxo-6,7-dihydro-3H-imidazo[4,5-f]indol-5-yl]-acetonitrile;
2-(1H-Indazol-3-yl)-7,7-dimethyl-5-(2-methylsulfanyl-ethyl)-5,7-dihydro-3H-imidazo[4,5-f]indol-6-one;
5-(2-Hydroxy-3-morpholin-4-yl-propyl)-2-(1H-indazol-3-yl)-7,7-dimethyl-5,7-dihydro-3H-imidazo[4,5-f]indol-6-one;
5-(Dimethyl-phosphinoylmethyl)-2-(1H-indazol-3-yl)-7,7-dimethyl-5,7-dihydro-3H-imidazo[4,5-f]indol-6-one;
5-(2-Hydroxy-ethyl)-2-(1H-indazol-3-yl)-7,7-dimethyl-5,7-dihydro-3H-imidazo[4,5-f]indol-6-one;
5-(2,3-Dihydroxy-propyl)-2-(1H-indazol-3-yl)-7,7-dimethyl-5,7-dihydro-3H-imidazo[4,5-f]indol-6-one;
5-(2-Amino-ethyl)-2-(1H-indazol-3-yl)-7,7-dimethyl-5,7-dihydro-3H-imidazo[4,5-f]indol-6-one;
2-(1H-Indazol-3-yl)-5-(2-methanesulfinyl-ethyl)-7,7-dimethyl-5,7-dihydro-3H-imidazo[4,5-f]indol-6-one; and
2-(1H-Indazol-3-yl)-5-(2-methanesulfonyl-ethyl)-7,7-dimethyl-5,7-dihydro-3H-imidazo[4,5-f]indol-6-one.

Another embodiment of the invention are the compounds of formula I, wherein:
(a) R¹ is alkyl, wherein said alkyl is substituted one or more times with alkyl-O—C(O)—, (alkyl)₃Si—O—, H₂N—C(S)—, HO—C(O)—, H₂N—C(O)—, alkyl-S(O)₂—NH— or phenyl-S(O)₂—NH—;
(b) R⁴, R⁵, R⁶ and R⁷ each represent hydrogen; and
(c) A is a single bond.

Such compounds, for example may be selected from the group consisting of:
[2-(1H-Indazol-3-yl)-7,7-dimethyl-6-oxo-6,7-dihydro-3H-imidazo[4,5-f]indol-5-yl]-acetic acid ethyl ester;
5-[2-(tert-Butyl-dimethyl-silanyloxy)-ethyl]-2-(1H-indazol-3-yl)-7,7-dimethyl-5,7-dihydro-3H-imidazo[4,5-f]indol-6-one;
2-[2-(1H-Indazol-3-yl)-7,7-dimethyl-6-oxo-6,7-dihydro-3H-imidazo[4,5-f]indol-5-yl]-thioacetamide;
[2-(1H-Indazol-3-yl)-7,7-dimethyl-6-oxo-6,7-dihydro-3H-imidazo[4,5-f]indol-5-yl]-acetic acid;
2-[2-(1H-Indazol-3-yl)-7,7-dimethyl-6-oxo-6,7-dihydro-3H-imidazo[4,5-f]indol-5-yl]-acetamide;
N-{2-[2-(1H-Indazol-3-yl)-7,7-dimethyl-6-oxo-6,7-dihydro-3H-imidazo[4,5-f]indol-5-yl]-ethyl}-benzenesulfonamide; compound with acetic acid; and
N-{2-[2-(1H-Indazol-3-yl)-7,7-dimethyl-6-oxo-6,7-dihydro-3H-imidazo[4,5-f]indol-5-yl]-ethyl}-methanesulfonamide.

Another embodiment of the invention are the compounds of formula I, wherein:
(a) R¹ is selected from the group consisting of:
   (1) arylalkyl, wherein the aryl is a mono- or bicyclic aromatic ring with 6 to 10 ring carbon atoms and wherein the aryl is optionally substituted one or more times with alkylsulfonyl; and
   (2) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(b) R⁴, R⁵, R⁶ and R⁷ each represent hydrogen; and
(c) A is a single bond.

Such compounds, for example, may be selected from the group consisting of:
2-(1H-Indazol-3-yl)-7,7-dimethyl-5-pyridin-3-ylmethyl-5,7-dihydro-3H-imidazo[4,5-f]indol-6-one;
5-Benzyl-2-(1H-indazol-3-yl)-7,7-dimethyl-5,7-dihydro-3H-imidazo[4,5-f]indol-6-one;
2-(1H-Indazol-3-yl)-5-(4-methanesulfonyl-benzyl)-7,7-dimethyl-5,7-dihydro-3H-imidazo[4,5-f]indol-6-one;
2-(1H-Indazol-3-yl)-7,7-dimethyl-5-thiazol-2-ylmethyl-5,7-dihydro-3H-imidazo[4,5-f]indol-6-one; and
2-(1H-Indazol-3-yl)-7,7-dimethyl-5-(1H-tetrazol-5-ylmethyl)-5,7-dihydro-3H-imidazo[4,5-f]indol-6-one.

Another embodiment of the invention are the compounds of formula I, wherein:
(a) R¹ is heterocyclyl-C(O)—(CH₂)ₙ—;
(b) R⁴, R⁵, R⁶ and R⁷ each represent hydrogen; and
(c) A is a single bond.

Such compounds are, for example, selected from the group consisting of:
2-(1H-Indazol-3-yl)-7,7-dimethyl-5-(2-morpholin-4-yl-2-oxo-ethyl)-5,7-dihydro-3H-imidazo[4,5-f]indol-6-one;
2-(1H-Indazol-3-yl)-7,7-dimethyl-5-[2-(4-methyl-piperazin-1-yl)-2-oxo-ethyl]-5,7-dihydro-3H-imidazo[4,5-f]indol-6-one; and
2-(1H-Indazol-3-yl)-7,7-dimethyl-5-(2-oxo-2-piperidin-1-yl-ethyl)-5,7-dihydro-3H-imidazo[4,5-f]indol-6-one.

Another embodiment of the invention are the compounds of formula I, wherein:
(a) R¹ is R⁸—NH—C(O)—(CH₂)ₙ—;
(b) R⁴, R⁵, R⁶ and R⁷ represent hydrogen; and
(c) A is a single bond.

Such compounds, for example, may be selected from the group consisting of:
N-(2-Dimethylamino-ethyl)-2-[2-(1H-indazol-3-yl)-7,7-dimethyl-6-oxo-6,7-dihydro-3H-imidazo[4,5-f]indol-5-yl]-acetamide;
N-Benzyl-2-[2-(1H-indazol-3-yl)-7,7-dimethyl-6-oxo-6,7-dihydro-3H-imidazo[4,5-f]indol-5-yl]-acetamide;
2-[2-(1H-Indazol-3-yl)-7,7-dimethyl-6-oxo-6,7-dihydro-3H-imidazo[4,5-f]indol-5-yl]-N-pyridin-3-ylmethyl-acetamide;
2-[2-(1H-Indazol-3-yl)-7,7-dimethyl-6-oxo-6,7-dihydro-3H-imidazo[4,5-f]indol-5-yl]-N-phenyl-acetamide;
N-(4-Fluoro-phenyl)-2-[2-(1H-indazol-3-yl)-7,7-dimethyl-6-oxo-6,7-dihydro-3H-imidazo[4,5-f]indol-5-yl]-acetamide;
N-(4-Fluoro-benzyl)-2-[2-(1H-indazol-3-yl)-7,7-dimethyl-6-oxo-6,7-dihydro-3H-imidazo[4,5-f]indol-5-yl]-acetamide;
N-(3,5-Dimethoxy-benzyl)-2-[2-(1H-indazol-3-yl)-7,7-dimethyl-6-oxo-6,7-dihydro-3H-imidazo[4,5-f]indol-5-yl]-acetamide;
N-(2,3-Dihydroxy-propyl)-2-[2-(1H-indazol-3-yl)-7,7-dimethyl-6-oxo-6,7-dihydro-3H-imidazo[4,5-f]indol-5-yl]-acetamide;
N-Hydroxy-2-[2-(1H-indazol-3-yl)-7,7-dimethyl-6-oxo-6,7-dihydro-3H-imidazo[4,5-f]indol-5-yl]-acetamide;
N-Benzyloxy-2-[2-(1H-indazol-3-yl)-7,7-dimethyl-6-oxo-6,7-dihydro-3H-imidazo[4,5-f]indol-5-yl]-acetamide; and
2-[2-(1H-Indazol-3-yl)-7,7-dimethyl-6-oxo-6,7-dihydro-3H-imidazo[4,5-f]indol-5-yl]-N-methoxy-acetamide.

Another embodiment of the invention are the compounds of formula I, wherein:
(a) R¹ is R⁹—C(O)—NH—(CH₂)ₙ—;
(b) R⁴, R⁵, R⁶ and R⁷ each represent hydrogen; and
(c) A is a single bond.

Such compounds are, for example, selected from the group consisting of:
N-{2-[2-(1H-Indazol-3-yl)-7,7-dimethyl-6-oxo-6,7-dihydro-3H-imidazo[4,5-f]indol-5-yl]-ethyl}-benzamide;
N-{2-[2-(1H-Indazol-3-yl)-7,7-dimethyl-6-oxo-6,7-dihydro-3H-imidazo[4,5-f]indol-5-yl]-ethyl}-2-phenyl-acetamide;
N-{2-[2-(1H-Indazol-3-yl)-7,7-dimethyl-6-oxo-6,7-dihydro-3H-imidazo[4,5-f]indol-5-yl]-ethyl}-nicotinamide;
Cyclopropanecarboxylic acid {2-[2-(1H-indazol-3-yl)-7,7-dimethyl-6-oxo-6,7-dihydro-3H-imidazo[4,5-f]indol-5-yl]-ethyl}-amide;
Morpholine-4-carboxylic acid {2-[2-(1H-indazol-3-yl)-7,7-dimethyl-6-oxo-6,7-dihydro-3H-imidazo[4,5-f]indol-5-yl]-ethyl}-amide;
Pyrrolidine-1-carboxylic acid {2-[2-(1H-indazol-3-yl)-7,7-dimethyl-6-oxo-6,7-dihydro-3H-imidazo[4,5-f]indol-5-yl]-ethyl}-amide;
4-Methyl-piperazine-1-carboxylic acid {2-[2-(1H-indazol-3-yl)-7,7-dimethyl-6-oxo-6,7-dihydro-3H-imidazo[4,5-f]indol-5-yl]-ethyl}-amide;
N-{2-[2-(1H-Indazol-3-yl)-7,7-dimethyl-6-oxo-6,7-dihydro-3H-imidazo[4,5-f]indol-5-yl]-ethyl}-acetamide; and
1-Benzyl-3-{2-[2-(1H-indazol-3-yl)-7,7-dimethyl-6-oxo-6,7-dihydro-3H-imidazo[4,5-f]indol-5-yl]-ethyl}-urea.

Another embodiment of the invention are the compounds of formula I, wherein:
(a) R¹ is hydrogen or alkyl;
(b) R⁴ and R⁷ each represent hydrogen;
(c) R⁵ is selected from the group consisting of:
   (1) hydrogen;
   (2) halogen;
   (3) cyano;
   (4) nitro;
   (5) amino;
   (6) carboxylic acid;
   (7) CH₃O—C(O)—;
   (8) H₂N—C(O)—;
   (9) CH₃O—N(CH₃)—C(O)—;
   (10) cycloalkyl-X—;
   (11) heterocyclyl-X—;
   (12) alkyl-X—, wherein the alkyl group is optionally substituted one or more times with halogen;
   (13) aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
   (14) arylalkyl-X—, wherein the aryl is a mono- or bicyclic aromatic ring with 6 to 10 ring carbon atoms and wherein the aryl is optionally substituted one or more times with halogen, (C₁-C₄)alkyl, (C₁-C₄)alkoxy, halogenated (C₁-C₄)alkoxy;
   (15) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms; and
   (16) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) R⁶ is hydrogen;
(e) X is —NH—, —O—, —S(O)₂NH—, —NHC(O)—, —C(O)—, —OC(O)NH— or —C(O)NH—; and
(f) A is a single bond.

Another embodiment of the invention are the compounds of formula I, wherein:
(a) R¹ is alkyl;
(b) R⁴ and R⁷ each represent hydrogen;
(c) R⁵ is halogen, cyano, nitro, amino, carboxylic acid, CH₃O—C(O)—, H₂N—C(O)— or CH₃O—N(CH₃)—C(O)—;
(d) R⁶ is hydrogen; and
(e) A is a single bond.

Such compounds are, for example, selected from the group consisting of:
5-Ethyl-2-(5-fluoro-1H-indazol-3-yl)-7,7-dimethyl-5,7-dihydro-3H-imidazo[4,5-f]indol-6-one;
2-(5-Chloro-1H-indazol-3-yl)-5-ethyl-7,7-dimethyl-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-1H-indazole-5-carboxylic acid;
5-Ethyl-7,7-dimethyl-2-(5-nitro-1H-indazol-3-yl)-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-1H-indazole-5-carbonitrile;
2-(5-Bromo-1H-indazol-3-yl)-5-ethyl-7,7-dimethyl-5,7-dihydro-3H-imidazo[4,5-f]indol-6-one;
3-(5-Isopropyl-7,7-dimethyl-6-oxo-3,5,6,7-tetrahydro-imidazo[4,5-f]indol-2-yl)-1H-indazole-5-carboxylic acid;
3-(5-Ethyl-7,7-dimethyl-6-oxo-3,5,6,7-tetrahydro-imidazo[4,5-f]indol-2-yl)-1H-indazole-5-carboxylic acid amide;
3-(5-Ethyl-7,7-dimethyl-6-oxo-3,5,6,7-tetrahydro-imidazo[4,5-f]indol-2-yl)-1H-indazole-5-carboxylic acid methyl ester;
3-(5-Ethyl-7,7-dimethyl-6-oxo-3,5,6,7-tetrahydro-imidazo[4,5-f]indol-2-yl)-1H-indazole-5-carboxylic acid methoxy-methyl-amide;
2-(5-Amino-1H-indazol-3-yl)-5-ethyl-7,7-dimethyl-5,7-dihydro-3H-imidazo[4,5-f]indol-6-one; and
2-(5-Amino-1H-indazol-3-yl)-5-isopropyl-7,7-dimethyl-5,7-dihydro-1H-imidazo[4,5-f]indol-6-one.

Another embodiment of the invention are the compounds of formula I, wherein:
(a) R¹ is alkyl;
(b) R⁴ and R⁷ each represent hydrogen;
(c) R⁵ is selected from the group consisting of:
   (1) alkyl-X, wherein the alkyl group is optionally substituted one or more times with halogen;
   (2) heterocyclyl-X—; and
   (3) aralkyl-X—, wherein the aryl is a mono- or bicyclic aromatic ring with 6 to 10 ring carbon atoms;
(d) R⁶ is hydrogen;
(e) X is —NH—, —O— or —C(O)—; and
(f) A is a single bond.

Such compounds, for example, may be selected from the group consisting of:
5-Ethyl-7,7-dimethyl-2-(5-trifluoromethoxy-1H-indazol-3-yl)-5,7-dihydro-3H-imidazo[4,5-f]indol-6-one;
5-Ethyl-7,7-dimethyl-2-[5-(piperidine-1-carbonyl)-1H-indazol-3-yl]-5,7-dihydro-3H-imidazo[4,5-f]indol-6-one;
5-Ethyl-7,7-dimethyl-2-[5-(4-methyl-piperazine-1-carbonyl)-1H-indazol-3-yl]-5,7-dihydro-3H-imidazo[4,5-f]indol-6-one;
5-Ethyl-7,7-dimethyl-2-[5-(morpholine-4-carbonyl)-1H-indazol-3-yl]-5,7-dihydro-3H-imidazo[4,5-f]indol-6-one;
2-[5-(4-Acetyl-piperazine-1-carbonyl)-1H-indazol-3-yl]-5-ethyl-7,7-dimethyl-5,7-dihydro-3H-imidazo[4,5-f]indol-6-one;
5-Ethyl-2-[5-(4-isopropyl-piperazine-1-carbonyl)-1H-indazol-3-yl]-7,7-dimethyl-5,7-dihydro-3H-imidazo[4,5-f]indol-6-one;
5-Ethyl-7,7-dimethyl-2-[5-(thiomorpholine-4-carbonyl)-1H-indazol-3-yl]-5,7-dihydro-3H-imidazo[4,5-f]indol-6-one;
5-Ethyl-7,7-dimethyl-2-[5-(thiazolidine-3-carbonyl)-1H-indazol-3-yl]-5,7-dihydro-3H-imidazo[4,5-f]indol-6-one;
5-Ethyl-2-[5-(4-methanesulfonyl-piperazine-1-carbonyl)-1H-indazol-3-yl]-7,7-dimethyl-5,7-dihydro-3H-imidazo[4,5-f]indol-6-one;
2-[5-(1,1-Dioxo-1λ⁶-thiomorpholine-4-carbonyl)-1H-indazol-3-yl]-5-ethyl-7,7-dimethyl-5,7-dihydro-3H-imidazo[4,5-f]indol-6-one;
5-Ethyl-7,7-dimethyl-2-[5-(1-oxo-1λ⁴-thiomorpholine-4-carbonyl)-1H-indazol-3-yl]-5,7-dihydro-3H-imidazo[4,5-f]indol-6-one;
2-(5-Acetyl-1H-indazol-3-yl)-5-ethyl-7,7-dimethyl-5,7-dihydro-3H-imidazo[4,5-f]indol-6-one;
2-(5-Benzylamino-1H-indazol-3-yl)-5-ethyl-7,7-dimethyl-5,7-dihydro-1H-imidazo[4,5-f]indol-6-one; and
2-(5-Benzyloxy-1H-indazol-3-yl)-5-ethyl-7,7-dimethyl-5,7-dihydro-1H-imidazo[4,5-f]indol-6-one.

Another embodiment of the invention are the compounds of formula I, wherein:
(a) R¹ is hydrogen or alkyl;
(b) R⁴ and R⁷ each represent hydrogen;
(c) R⁵ is selected from the group consisting of:
   (1) alkyl-X—;
   (2) aryl-X—, wherein the aryl is a mono- or bicyclic aromatic ring with 6 to 10 ring carbon atoms;
   (3) arylalkyl-X—, wherein the aryl is a mono- or bicyclic aromatic ring with 6 to 10 ring carbon atoms and wherein the aryl is optionally substituted one or more times with halogen or halogenated (C₁-C₄)alkoxy; and
   (4) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) R⁶ is hydrogen;
(e) X is —NHC(O)—; and
(f) A is a single bond.

Such compounds, for example, are selected from the group consisting of:
3-(7,7-Dimethyl-6-oxo-3,5,6,7-tetrahydro-imidazo[4,5-f]indol-2-yl)-1H-indazole-5-carboxylic acid ethylamide;
3-(7,7-Dimethyl-6-oxo-3,5,6,7-tetrahydro-imidazo[4,5-f]indol-2-yl)-1H-indazole-5-carboxylic acid benzylamide;
3-(7,7-Dimethyl-6-oxo-3,5,6,7-tetrahydro-imidazo[4,5-f]indol-2-yl)-1H-indazole-5-carboxylic acid phenylamide;
3-(5-Ethyl-7,7-dimethyl-6-oxo-3,5,6,7-tetrahydro-imidazo[4,5-f]indol-2-yl)-1H-indazole-5-carboxylic acid benzylamide;
3-(5-Ethyl-7,7-dimethyl-6-oxo-3,5,6,7-tetrahydro-imidazo[4,5-f]indol-2-yl)-1H-indazole-5-carboxylic acid (pyridin-2-ylmethyl)-amide;
3-(5-Ethyl-7,7-dimethyl-6-oxo-3,5,6,7-tetrahydro-imidazo[4,5-f]indol-2-yl)-1H-indazole-5-carboxylic acid (pyridin-3-ylmethyl)-amide; compound with acetic acid;
3-(5-Ethyl-7,7-dimethyl-6-oxo-3,5,6,7-tetrahydro-imidazo[4,5-f]indol-2-yl)-1H-indazole-5-carboxylic acid (pyridin-4-ylmethyl)-amide;
3-(5-Ethyl-7,7-dimethyl-6-oxo-3,5,6,7-tetrahydro-imidazo[4,5-f]indol-2-yl)-1H-indazole-5-carboxylic acid phenylamide;
3-(5-Ethyl-7,7-dimethyl-6-oxo-3,5,6,7-tetrahydro-imidazo[4,5-f]indol-2-yl)-1H-indazole-5-carboxylic acid ethylamide;
3-(5-Ethyl-7,7-dimethyl-6-oxo-3,5,6,7-tetrahydro-imidazo[4,5-f]indol-2-yl)-1H-indazole-5-carboxylic acid 2,4-difluoro-benzylamide;
3-(5-Ethyl-7,7-dimethyl-6-oxo-3,5,6,7-tetrahydro-imidazo[4,5-f]indol-2-yl)-1H-indazole-5-carboxylic acid 3-trifluoromethoxy-benzylamide;
3-(5-Ethyl-7,7-dimethyl-6-oxo-3,5,6,7-tetrahydro-imidazo[4,5-f]indol-2-yl)-1H-indazole-5-carboxylic acid 4-difluoromethoxy-benzylamide;
3-(5-Ethyl-7,7-dimethyl-6-oxo-3,5,6,7-tetrahydro-imidazo[4,5-f]indol-2-yl)-1H-indazole-5-carboxylic acid 3-chloro-benzylamide; and
3-(5-Ethyl-7,7-dimethyl-6-oxo-3,5,6,7-tetrahydro-imidazo[4,5-f]indol-2-yl)-1H-indazole-5-carboxylic acid 4-trifluoromethoxy-benzylamide.

Another embodiment of the invention are the compounds of formula I, wherein:
(a) R¹ is alkyl;
(b) R⁴ and R⁷ each represent hydrogen;
(c) R⁵ is selected from the group consisting of:
   (1) cycloalkyl-X—;
   (2) heterocyclyl-X—;
   (3) alkyl-X—;
   (4) aryl-X—, wherein the aryl is a mono- or bicyclic aromatic ring with 6 to 10 ring carbon atoms and wherein the aryl is optionally substituted one or more times with halogen;
   (5) arylalkyl-X—, wherein the aryl is a mono- or bicyclic aromatic ring with 6 to 10 ring carbon atoms and wherein the aryl is optionally substituted one or more times with halogen;
   (6) (C₁-C₄)alkyl;
   (7) (C₁-C₄)alkoxy; and
   (8) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) R⁶ is hydrogen;
(e) X is —C(O)NH—; and
(f) A is a single bond.

Such compounds, for example, may be selected from the group consisting of:
N-[3-(5-Ethyl-7,7-dimethyl-6-oxo-1,5,6,7-tetrahydro-imidazo[4,5-f]indol-2-yl)-1H-indazol-5-yl]-2-o-tolyl-acetamide;
N-[3-(5-Ethyl-7,7-dimethyl-6-oxo-3,5,6,7-tetrahydro-imidazo[4,5-f]indol-2-yl)-1H-indazol-5-yl]-2-phenyl-acetamide;
N-[3-(5-Ethyl-7,7-dimethyl-6-oxo-1,5,6,7-tetrahydro-imidazo[4,5-f]indol-2-yl)-1H-indazol-5-yl]-isonicotinamide;
Pyridine-2-carboxylic acid [3-(5-ethyl-7,7-dimethyl-6-oxo-1,5,6,7-tetrahydro-imidazo[4,5-f]indol-2-yl)-1H-indazol-5-yl]-amide;
N-[3-(5-Ethyl-7,7-dimethyl-6-oxo-1,5,6,7-tetrahydro-imidazo[4,5-f]indol-2-yl)-1H-indazol-5-yl]-2-p-tolyl-acetamide;
2-(3,5-Dimethoxy-phenyl)-N-[3-(5-ethyl-7,7-dimethyl-6-oxo-1,5,6,7-tetrahydro-imidazo[4,5-f]indol-2-yl)-1H-indazol-5-yl]-acetamide;
N-[3-(5-Ethyl-7,7-dimethyl-6-oxo-1,5,6,7-tetrahydro-imidazo[4,5-f]indol-2-yl)-1H-indazol-5-yl]-4-fluoro-benzamide;
N-[3-(5-Ethyl-7,7-dimethyl-6-oxo-1,5,6,7-tetrahydro-imidazo[4,5-f]indol-2-yl)-1H-indazol-5-yl]-2-(4-fluoro-phenyl)-acetamide;
N-[3-(5-Ethyl-7,7-dimethyl-6-oxo-1,5,6,7-tetrahydro-imidazo[4,5-f]indol-2-yl)-1H-indazol-5-yl]-nicotinamide;
N-[3-(5-Ethyl-7,7-dimethyl-6-oxo-1,5,6,7-tetrahydro-imidazo[4,5-f]indol-2-yl)-1H-indazol-5-yl]-propionamide;
Cyclopropanecarboxylic acid [3-(5-ethyl-7,7-dimethyl-6-oxo-1,5,6,7-tetrahydro-imidazo[4,5-f]indol-2-yl)-1H-indazol-5-yl]-amide;
N-[3-(5-Ethyl-7,7-dimethyl-6-oxo-1,5,6,7-tetrahydro-imidazo[4,5-f]indol-2-yl)-1H-indazol-5-yl]-benzamide;
Cyclohexanecarboxylic acid [3-(5-ethyl-7,7-dimethyl-6-oxo-1,5,6,7-tetrahydro-imidazo[4,5-f]indol-2-yl)-1H-indazol-5-yl]-amide;
4-Methyl-piperazine-1-carboxylic acid [3-(5-ethyl-7,7-dimethyl-6-oxo-3,5,6,7-tetrahydro-imidazo[4,5-f]indol-2-yl)-1H-indazol-5-yl]-amide;
Piperidine-1-carboxylic acid [3-(5-ethyl-7,7-dimethyl-6-oxo-1,5,6,7-tetrahydro-imidazo[4,5-f]indol-2-yl)-1H-indazol-5-yl]-amide;
Morpholine-4-carboxylic acid [3-(5-ethyl-7,7-dimethyl-6-oxo-1,5,6,7-tetrahydro-imidazo[4,5-f]indol-2-yl)-1H-indazol-5-yl]-amide;
Pyrrolidine-1-carboxylic acid [3-(5-ethyl-7,7-dimethyl-6-oxo-1,5,6,7-tetrahydro-imidazo[4,5-f]indol-2-yl)-1H-indazol-5-yl]-amide;
4-Methyl-piperazine-1-carboxylic acid [3-(5-ethyl-7,7-dimethyl-6-oxo-1,5,6,7-tetrahydro-imidazo[4,5-f]indol-2-yl)-1H-indazol-5-yl]-amide;
N-[3-(5-Ethyl-7,7-dimethyl-6-oxo-3,5,6,7-tetrahydro-imidazo[4,5-f]indol-2-yl)-1H-indazol-5-yl]-acetamide; and
4-Acetyl-piperazine-1-carboxylic acid [3-(5-ethyl-7,7-dimethyl-6-oxo-1,5,6,7-tetrahydro-imidazo[4,5-f]indol-2-yl)-1H-indazol-5-yl]-amide.

Another embodiment of the invention are the compounds of formula I, wherein:
(a) R¹ is alkyl;
(b) R⁴ and R⁷ each represent hydrogen;
(c) R⁵ is aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
(d) R⁶ is hydrogen;
(e) X is —S(O)₂NH—; and
(f) A is a single bond.

Such compounds, for example, may be selected from the group consisting of:
N-[3-(5-Ethyl-7,7-dimethyl-6-oxo-1,5,6,7-tetrahydro-imidazo[4,5-f]indol-2-yl)-1H-indazol-5-yl]-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-4-methoxy-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-2-nitro-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-3-methoxy-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-2-trifluoromethoxy-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-4-fluoro-benzenesulfonamide;
3-Chloro-N-[3-(5-ethyl-7,7-dimethyl-6-oxo-1,5,6,7-tetrahydro-imidazo[4,5-f]indol-2-yl)-1H-indazol-5-yl]-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-3-methyl-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-2-methanesulfonyl-benzenesulfonamide;
N-[3-(5-Ethyl-7,7-dimethyl-6-oxo-3,5,6,7-tetrahydro-imidazo[4,5-f]indol-2-yl)-1H-indazol-5-yl]-2,5-difluoro-benzenesulfonamide;
4-Fluoro-N-[3-(5-isopropyl-7,7-dimethyl-6-oxo-1,5,6,7-tetrahydro-imidazo[4,5-f]indol-2-yl)-1H-indazol-5-yl]-benzenesulfonamide;
N-[3-(5-Isopropyl-7,7-dimethyl-6-oxo-1,5,6,7-tetrahydro-imidazo[4,5-f]indol-2-yl)-1H-indazol-5-yl]-2-methanesulfonyl-benzenesulfonamide; and
N-[3-(5-Isopropyl-7,7-dimethyl-6-oxo-1,5,6,7-tetrahydro-imidazo[4,5-f]indol-2-yl)-1H-indazol-5-yl]-2-nitro-benzenesulfonamide.

Another embodiment of the invention are the compounds of formula I, wherein:
(a) R¹ is alkyl;
(b) R⁴ and R⁷ each represent hydrogen;
(c) R⁵ is arylalkyl-X—, wherein the aryl is a mono- or bicyclic aromatic ring with 6 to 10 ring carbon;
(d) R⁶ is hydrogen;
(e) X is —OC(O)NH—; and
(f) A is a single bond.

Such a compound is:
[3-(5-Ethyl-7,7-dimethyl-6-oxo-1,5,6,7-tetrahydro-imidazo[4,5-f]indol-2-yl)-1H-indazol-5-yl]-carbamic acid benzyl ester.

Another embodiment of the invention are the compounds of formula I, wherein:
(a) R¹ is alkyl;
(b) R⁴ and R⁷ represent hydrogen;
(c) R⁵ is hydrogen;
(d) R⁶ is selected from the group consisting of:
   (1) halogen;
   (2) carboxylic acid;
   (3) H₂N—C(O)—;
   (4) alkyl-X—;
   (5) aryl-X—, wherein the aryl is a mono- or bicyclic aromatic ring with 6 to 10 ring carbon atoms; and
   (6) arylalkyl-X—, wherein the aryl is a mono- or bicyclic aromatic ring with 6 to 10 ring carbon atoms;
(e) X is —NHC(O)—; and
(f) A is a single bond.

Such compounds, for example, may be selected from the group consisting of:
2-(6-Bromo-1H-indazol-3-yl)-5-ethyl-7,7-dimethyl-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-1H-indazole-6-carboxylic acid;
3-(7,7-Dimethyl-6-oxo-3,5,6,7-tetrahydro-imidazo[4,5-f]indol-2-yl)-1H-indazole-6-carboxylic acid benzylamide;
3-(7,7-Dimethyl-6-oxo-3,5,6,7-tetrahydro-imidazo[4,5-f]indol-2-yl)-1H-indazole-6-carboxylic acid ethylamide;
3-(7,7-Dimethyl-6-oxo-3,5,6,7-tetrahydro-imidazo[4,5-f]indol-2-yl)-1H-indazole-6-carboxylic acid phenylamide;
3-(5-Ethyl-7,7-dimethyl-6-oxo-3,5,6,7-tetrahydro-imidazo[4,5-f]indol-2-yl)-1H-indazole-6-carboxylic acid ethylamide;
3-(5-Ethyl-7,7-dimethyl-6-oxo-3,5,6,7-tetrahydro-imidazo[4,5-f]indol-2-yl)-1H-indazole-6-carboxylic acid phenylamide;
3-(5-Ethyl-7,7-dimethyl-6-oxo-3,5,6,7-tetrahydro-imidazo[4,5-f]indol-2-yl)-1H-indazole-6-carboxylic acid benzylamide; and
3-(5-Ethyl-7,7-dimethyl-6-oxo-3,5,6,7-tetrahydro-imidazo[4,5-f]indol-2-yl)-1H-indazole-6-carboxylic acid amide.

Another embodiment of the invention are the compounds of formula I, wherein A is —CH₂—.

Another embodiment of the invention are the compounds of formula I, wherein:
(a) R¹ is hydrogen or alkyl;
(b) R⁴, R⁵, R⁶ and R⁷ represent hydrogen; and
(c) A is —CH₂—.

Such compounds, for example, may be selected from the group consisting of:
2-(1H-Indazol-3-yl)-8,8-dimethyl-1,5,7,8-tetrahydro-imidazo[4,5-g]quinolin-6-one; and
5-Ethyl-2-(1H-indazol-3-yl)-8,8-dimethyl-3,5,7,8-tetrahydro-imidazo[4,5-g]quinolin-6-one.

Another embodiment of the invention is a process for the preparation of the compounds of formula I, wherein:
a) a compound of formula II 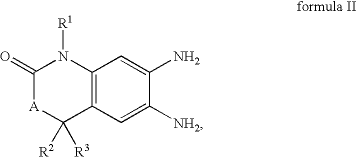
   wherein R¹ to R³ and A have the significance given above for formula I; is reacted with a compound of formula III, 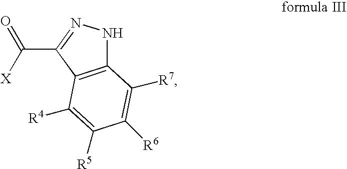
   wherein X is —OH, —Cl, —H or —OMe and R⁴ to R⁷ have the significance given above for formula I;
   to give the compounds of formula I, 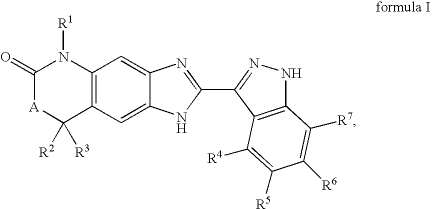
   wherein R¹ to R⁷ and A have the significance given above for formula I;
b) optionally said compound of formula I is isolated from the reaction mixture, and
c) optionally converted into a pharmaceutically acceptable salt or ester.

The tricyclic compounds of formula I, or a pharmaceutically acceptable salt or ester thereof, which are subject of the present invention, may be prepared by any process known to be applicable to the preparation of chemically-related compounds. Such processes, when used to prepare a compound of the formula I, or a pharmaceutically-acceptable salt or ester thereof, are illustrated by the following representative schemes 1 and 2 and examples in which, unless otherwise stated, A, R¹, R², R³, R⁴, R⁵, R⁶ and R⁷ have the significance given herein before for formula I. Necessary starting materials may be obtained by standard procedures of organic chemistry. The preparation of such starting materials is described within the accompanying examples or in the literature cited below with respect to scheme 1 to 4. Alternatively necessary starting materials are obtainable by analogous procedures to those illustrated which are within the ordinary skill of an organic chemist.

The imidazole ring system of formula I can be formed by different synthetic pathways in analogy to methods described in the literature (e.g. see Mertens, A., et al., J. Med. Chem. 30 (1987) 1279-1287 and U.S. Pat. No. 4,695,567A).

One route for the preparation of compounds of formula I (Scheme 1) starts from diamines of formula II which can be reacted with carboxylic acids (compounds of formula III with X═OH), acid chlorides (X═Cl), aldehydes (X═H), methyl carboxylates (X═OMe) or activated esters (X=e.g. hydroxybenzotriazole). For detailed procedures see the literature cited above.

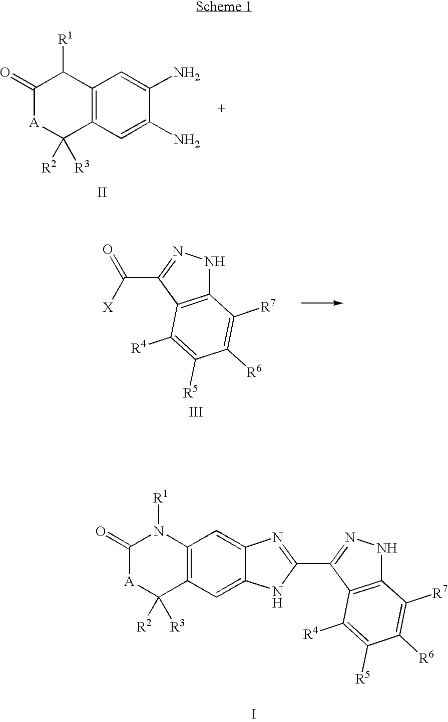

In scheme 1, R¹, R², R³, R⁴, R⁵, R⁶, R⁷ and A have the significance as given above for formula I. The synthesis of the corresponding diamines of formula II or precursors thereof is described in Mertens, A., et al., J. Med. Chem. 30 (1987) 1279-1287, von der Saal, W., et al. J. Med. Chem. 32 (1989) 1481-1491, U.S. Pat. No. 4,666,923A, U.S. Pat. No. 4,695,567A, U.S. Pat. No. 4,863,945A and U.S. Pat. No. 4,985,448A. For instance, the diamines of formula II, wherein A is a single bond are named IIa and can be synthesized according to U.S. Pat. No. 4,666,923A, DE 34 10 168 and Mertens, A., et al., J. Med. Chem. 30 (1987) 1279-1287 as shown in scheme 2:

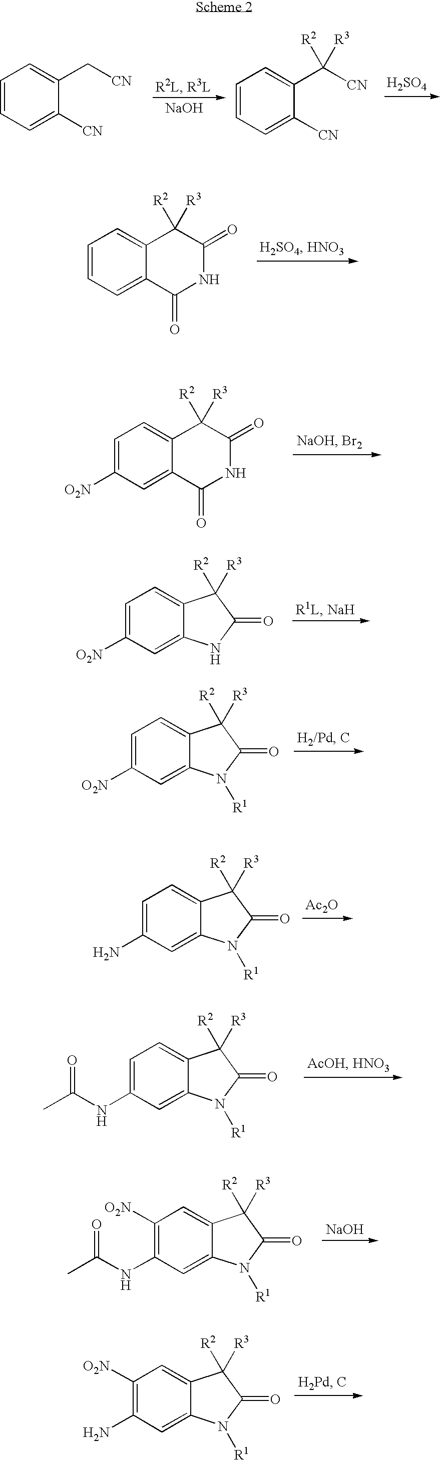

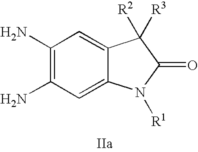

In scheme 2, R² and R³ represent alkyl, R¹ is an optionally substituted alkyl and L represents a leaving group such as iodine, bromine, chlorine, or triflate or the like.

In an alternative procedure diamines of formula Ia can be obtained by an alkylation of diamines of formula Ilb as shown in scheme 2a. Diamines of formula lIb can be synthesized according to scheme 2 under omission of the fifth step.

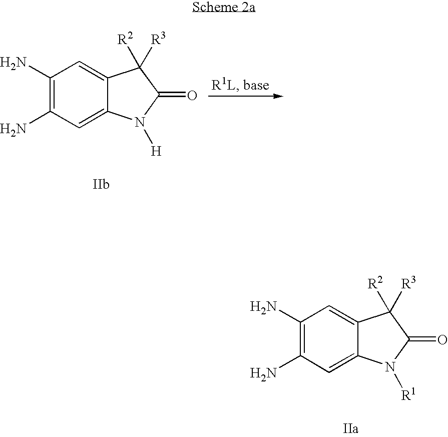

In scheme 2a, R² and R³ represent alkyl, R¹ is an optionally substituted alkyl and L represents a leaving group as e.g. iodine, bromine, chlorine, triflate and the like. The alkylation reaction is typically carried out in the presence of a base such as sodium hydride, potassium hydride and the like, especially sodium hydride, in inert solvents such as dimethylformamide (DMF), N-methyl-pyrrolidinone (NMP), tetrahydrofuran and the like.

Indazoles of formula III in scheme 1 are either commercially available or they can be prepared by different synthetic routes according to the nature of “X”. If “X” is hydroxy the corresponding 3-indazolecarboxylic acids are named IIIa and can be manufactured as shown in the following scheme 3:

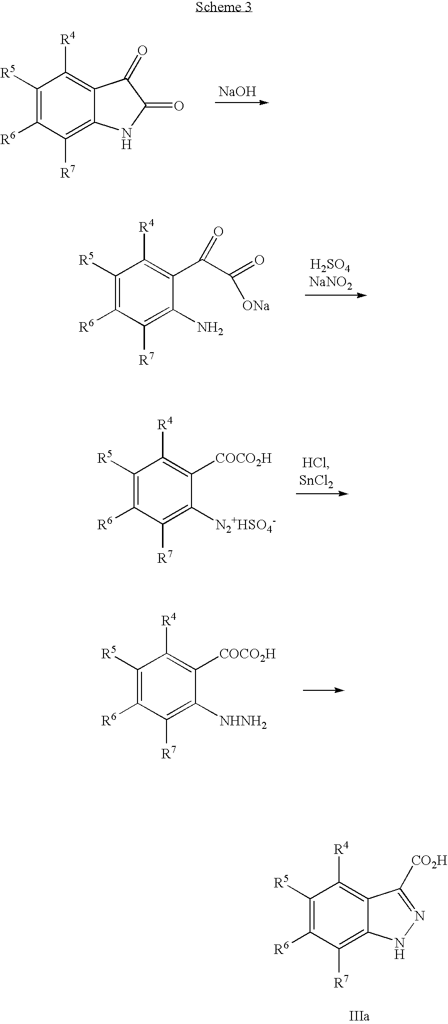

In scheme 3, R⁴, R⁵, R⁶ and R⁷ have the significance as given above for formula I. As described in Snyder, H. R., et al., J. Am. Chem. Soc. (1952) 2009-2012, 3-indazolecarboxylic acids of formula IIIa can be prepared from isatins by basic ring opening, followed by diazotation of the amino group, reduction to the hydrazine and condensation to give the desired indazole.

The necessary isatins are either commercially available or may be obtained by standard procedures of organic chemistry, e.g. by reaction of the corresponding aniline with oxalylchloride. The reaction starts with an N-acylation, followed by an intramolecular acylation which can be catalyzed by Lewis acids. (e.g. Piggott, M. J. and Wege, D., Australian Journal of Chemistry 53 (2000) 749-754; March, J., Advanced Organic Chemistry 4th ed. (1992) 539-542) More often the corresponding aniline is reacted with chloral hydrate (2,2,2-trichlor-1,1-ethanediol) and hydroxylamine (hydrochloride) (via the hydroxyiminoacetamides) in a cyclization reaction to the desired isatins (e.g. Sheibley, F. E., and McNulty, J. S., J. Org. Chem. 21 (1956) 171-173; Lisowski, V., et al., J. Org. Chem. 65 (2000) 4193-4194).

If “X” is hydrogen the corresponding 1H-Indazole-3-carbaldehydes are named IIIb and can be manufactured e.g. as shown in the following scheme 4.

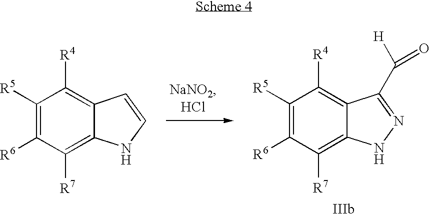

In scheme 4, R⁴, R⁵, R⁶ and R⁷ have the significance as given above for formula I. The compounds of formula IIIb can be synthesized from suitably substituted indoles by treatment with NaNO₂/HCl as described e.g. in Sall, D. J., et al., J. Med. Chem. 40 (1997) 2843-2857.

Certain substituents on the groups R¹, R⁵ and R⁶ may not be inert to the conditions of the synthesis sequences described above and may require protection by standard protecting groups known in the art. For instance, an amino or hydroxyl group may be protected as an acetyl or tert.-butoxycarbonyl derivative. Alternatively, some substituents may be derived from others at the end of the reaction sequence. For instance, a compound of formula I may be synthesized bearing a nitro-, an ethoxycarbonyl, an ether, a sulfonic acid substituent on the group R⁵ and R⁶, which substituents are finally converted to an amino- (e.g. by reduction of a nitro group or cleavage of a suitable amino protection group (e.g. removal of a Boc group with TFA)), alkylamino- (e.g. by reductive amination of an amino group), dialkylamino- (e.g. by alkylation of an amino group, reduction of an appropriate acylamino group with lithium aluminum hydride or Eschweiler-Clarke reaction with an appropriate amino or alkylamino group), acylamino- (by amide formation from an amino group e.g. with appropriate acyl halides or with appropriate carboxylic acids after their activation with CDI, EDC etc.), alkylsulfonylamino (e.g. by reaction of an amino group with sulfonyl chlorides), arylsulfonylamino substituent (e.g. by reaction of an amino group with sulfonyl chlorides), hydroxyl- (by cleavage of a suitable hydroxy protection group (e.g. hydrogenolytic removal of a benzyl ether or oxidative cleavage of a p-methoxy benzyl ether), ether- (e.g. by Williamson's ether synthesis from a hydroxyl group) or to a carboxamide substituent (e.g. by amide formation from a carboxylic acid group with appropriate amines after activation of the carboxylic acid group with CDI, EDC etc. or conversion to an acyl chloride), or to a sulfonamide substituent by standard procedures.

Pharmaceutical compositions containing a compound of the present invention or a pharmaceutically acceptable salt or ester thereof and a therapeutically inert carrier are an object of the present invention, as is a process for their production, which comprises bringing one or more compounds of the present invention and/or pharmaceutically acceptable salts or esters and, if desired, one or more other therapeutically valuable substances into a galenical administration form together with one or more therapeutically inert carriers.

In accordance with the invention the compounds of the present invention as well as their pharmaceutically acceptable salts or esters are useful in the control or prevention of illnesses. Based on their Aurora tyrosine kinase inhibition and their antiproliferative activity, said compounds are useful for the treatment of diseases such as cancer in humans or animals and for the production of corresponding pharmaceutical compositions. The dosage depends on various factors such as manner of administration, species, age and/or individual state of health.

An embodiment of the invention is a pharmaceutical composition, containing one or more compounds according to formula I, together with pharmaceutically acceptable excipients.

Another embodiment of the invention is a pharmaceutical composition containing one or more compounds of formula I as active ingredients together with pharmaceutically acceptable adjuvants for the treatment of diseases mediated by an inappropriate activation of Aurora family tyrosine kinases.

Another embodiment of the invention is a pharmaceutical composition, containing one or more compounds according to formula I, for the inhibition of tumor growth.

Another embodiment of the invention is a pharmaceutical composition containing one or more compounds of formula I as active ingredients together with pharmaceutically acceptable adjuvants for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is a pharmaceutical composition containing one or more compounds of formula I as active ingredients together with pharmaceutically acceptable adjuvants for the treatment of acute-myelogenous leukemia (AML, acute lymphocytic leukemia (ALL) and gastrointestinal stromal tumor (GIST).

Another embodiment of the invention is the use of one or more compounds of formula I for the manufacture of pharmaceutical compositions for the treatment of diseases mediated by an inappropriate activation of Aurora family tyrosine kinases.

Another embodiment of the invention is the use of a compound according to formula I, for the manufacture of corresponding pharmaceutical compositions for the inhibition of tumor growth.

Another embodiment of the invention is the use of a compound according to formula I, for the manufacture of corresponding pharmaceutical compositions for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is the use of a compound according to formula I, for the treatment of acute-myelogenous leukemia (AML, acute lymphocytic leukemia (ALL) and gastrointestinal stromal tumor (GIST).

Another embodiment of the invention is the use of the compounds of formula I as Aurora A tyrosine kinase inhibitors.

Another embodiment of the invention is the use of the compounds of formula I as anti-proliferating agents.

Another embodiment of the invention is the use of one or more compounds of formula I for the treatment of cancer.

Pharmacological Activity

The compounds of formula I and their pharmaceutically acceptable salts or esters possess valuable pharmacological properties. It has been found that said compounds show activity as inhibitors of the Aurora kinase family and also show anti-proliferative activity. Consequently the compounds of the present invention are useful in the therapy and/or prevention of illnesses with known over-expression of kinases of the Aurora family preferably Aurora A, especially in the therapy and/or prevention of illnesses mentioned above. The activity of the present compounds as inhibitors of the Aurora kinase family is demonstrated by the following biological assay:
IC₅₀ determination for inhibitors of Aurora A
(96 MTP-ELISA)
   Assay Principle

Aurora A is a serine threonine kinase involved in spindle assembly and chromosome segregation. The assay is a typically ELISA-type assay where biotinylated substrate (PKB-GSK2) is phosphorylated. Phosphorylation is detected by peroxidase (POD) labelled polyclonal antibody (PAK<M-Ig>S-IgG-POD) and phosphopeptide monoclonal antibody (Mab) (MAK<P-GSK>M-27E5-IgG). The assay is validated for IC₅₀-determination.

Materials

Assay plates: 96-well polystyrene plates, streptavidin-coated,

Samples: 10 mM in dimethylsulfoxide (DMSO)

Aurora A-His-4: C-terminally Histidine₄ (His₄)-tagged Aurora A full-length protein, stock solution 0.7 mg/ml, final conc.: 250 ng/ml

PKB-GSK2: biotinylated peptide derived from human GSK3-alpha sequence (Biotin-SGRARTSSFAEPGG-CONH₂), stock solution 600 μM, final conc.: 200 nM

PAK<M-Ig>S-IgG-POD: Anti-mouse IgG, horse radish peroxidase(HRP)-linked Antibody, diluted in 3% BSA/PBS-T (1:10000), (Cell Signaling, Cat. No.: 7076)

MAK<P-GSK>M-27E5-IgG: Phospho-GSK-3-alpha (Ser 21) (27E5) Monoclonal Antibody, stock solution 1.85 mg/ml, diluted in 3% BSA/PBS-T (1:6000), final conc.: 0.31 μg/ml, (Cell Signaling, Cat. No.: 9337B)

ATP: Adenosine-5′-triphosphate 1 mM, diluted in kinase buffer, (Roche Diagnostics GmbH, Cat. No.: 127531-001,), final conc.: 4 μM

TRIS: 2-Amino-2-hydroxymethyl-1,3-propoanediol (“tris-(hydroxymethyl)-aminomethane”) (MERCK, Cat. No.: 108382.2500)

BSA: Bovine Serum Albumin Fraction V, fatty acid free (Roche Diagnostics GmbH, Cat. No. 9100221)

EDTA Titriplex III (di-Sodium-EDTA di-Hydrate), 120 mM, (MERCK, Cat. No.: 1.08418.1000)

ABTS buffer: ABTS (2,2′-azino-bis(3-ethylbenzthiazoline-6-sulfonic acid)) 16.7 mg/ml (Roche Diagnostics GmbH, Cat. No.: 1204530)
ABTS tablets: dissolve one ABTS tablet in 50 ml of working solution (ABTS buffer) (Roche Diagnostics GmbH, Cat. No.: 1112422 )
Tween 20: Polysorbat 20 (Roche Diagnostics GmbH, Cat. No.: 10006394-001)
DTT: 1,4-Dithiothreitol (Roche Diagnostics GmbH, Cat. No.: 197777)
MgCl₂×6H₂O MERCK, Cat. No.: 105833.1000
Kinase buffer: 50 mM TRIS, 10 mM MgCl₂, 1 mM DTT, 0.1% Tween 20, pH 7.8 PBS-T (=Wash buffer) (PBS-T)10 g/l PBS(Phosphate buffered saline) with 0.033% Tween 20
3% BSA/PBS-T: 3% BSA dissolved in PBS-T
   Method

This assay is performed in 96-well format for IC₅₀ determination with 5 samples (each with 8 concentrations by twofold testing), 100 μl incubation volume and the following plate layout:

| | 1 | 2 | 3 | 4 | 5 | 6 | 7 | 8 | 9 | 10 | 11 | 12 |
|---|---|---|---|---|---|---|---|---|---|---|---|---|
| A | NC | RS a | RS a | S1a | S1a | S2a | S2a | NC | S3a | S3a | S4a | S4a |
| B | NC | RS b | RS b | S1b | S1b | S2b | S2b | NC | S3b | S3b | S4b | S4b |
| C | NC | RS c | RS c | S1c | S1c | S2c | S2c | NC | S3c | S3c | S4c | S4c |
| D | NC | RS d | RS d | S1d | S1d | S2d | S2d | NC | S3d | S3d | S4d | S4d |
| E | PC | RS e | RS e | S1e | S1e | S2e | S2e | PC | S3e | S3e | S4e | S4e |
| F | PC | RS f | RS f | S1f | S1f | S2f | S2f | PC | S3f | S4f | S4f | S4f |
| G | PC | RS g | RS g | S1g | S1g | S2g | S2g | PC | S3g | S4g | S4g | S4g |
| H | PC | RS h | RS h | S1h | S1h | S2h | S2h | PC | S3h | S4h | S4h | S4h |

NC negative control, without ATP, 1% DMSO
PC positive control, with ATP, 1% DMSO

S samples, with ATP, 1% DMSO, final conc.: a=100 μM, b=20 μM, c=4 μM, d=0.8 μM, e=0.16 μM, f=0.032 μM, g=0.0064 μM, h=0.00128 μM
Step/Action
(1) Sample preparation: add 24 μl per well samples (descending sequence) diluted in kinase buffer to assay plate (final conc. for DMSO 1%).
2. Add directly 16 μl Aurora-A-his-4 diluted in kinase buffer to assay plate.
3. Add directly 40 μl per well PKB-GSK2/ATP mixture to assay plate, (final conc.: Aurora A=250 ng/ml, GSK2=200 nM, ATP=4 μM). Negative control: without ATP.
4. Incubate assay plate for exactly 90 min at room temperature.
5. Stop reaction by adding 20 μl EDTA in all wells.
6. Wash assay plate 3× with 200 μl washing buffer per well.
7. Add 100 μl MAK<P-GSK>M27E5-IgG (1:10000) and PAK<M-Ig>S-IgG-POD (1:6000) dissolved in 3% BSA/PBS-T to assay plate per well.
8. Incubate assay plate for 60 min at room temperature.
9. Wash assay plate 3× with 200 μl washing buffer per well
10. Add 100 μl ABTS solution to assay plate per well, incubate for approx. 4 min at RT on MTP shaker.
11. Measure absorption at 405/492 nm.
12. Calculate % inhibition as:
   (1−(Eₛₐₘₚₗₑ−E_{NC})/(E_{PC}−E_{NC}))×100 1)

13. Calculate IC₅₀ using a non-linear curve fit (XLfit software (ID Business Solution Ltd., Guilford, Surrey, UK))

TABLE 1
Results

| | IC50 Aurora |
|---|---|
| | A kinase |
| Example No. | inhibition [nM] |
| 7 | 14 |
| 6 | 28 |
| 2, 3, 5, 8, 15, 16, 17, 18, 19, 21, 23, 24, | 1-100 |
| 27, 28, 29, 30, 33, 34, 35, 36, 37, 38, | |
| 39, 40, 42, 43, 44, 48, 49, 52, 53, | |
| 9, 10, 11, 13, 26 | 100-250 |

Antiproliferative Activity

The activity of the present compounds as antiproliferative agents is demonstrated by the following biological assay:

Viability Assay—HCT 116 Cells

A viability assay was performed using the CellTiter-Glo® Luminescent Cell Viability Assay (see Promega Corporation's Technical Bulletin No. 288, pp. 1-11 [revised 2/04] which is hereby incorporated by reference in its entirety). This assay is a homogeneous method of determining the number of viable cells in culture based on quantitation of the ATP present, an indicator of metabolically active cells. The assay is designed for use with multiwell formats, making it ideal for automated high-throughput screening (HTS), cell proliferation and cytotoxicity assays. The homogeneous assay procedure involves adding a single reagent (containing luciferase, luciferan substrate, and buffer) directly to cells cultured in serum-supplemented medium. Cell washing, removal of medium and multiple pipetting steps are not required. The system detects as few as 15 cells/well in a 384-well format in 10 minutes after adding reagent and mixing.

The homogeneous “add-mix-measure” format results in cell lysis and generation of a luminescent signal proportional to the amount of ATP present. The amount of ATP is directly proportional to the number of cells present in culture. The above-referenced assay generates a “glow-type” luminescent signal, produced by the luciferase reaction, which has a half-life generally greater than five hours, depending on cell type and medium used. The extended half-life eliminates the need to use reagent injectors and provides flexibility for continuous or batch mode processing of multiple plates. The unique homogeneous format avoids errors that may be introduced by other ATP measurement methods that require multiple steps.

HCT 116 cells (human colon carcinoma, ATCC-No. CCl-247) were cultivated in RPMI 1640 medium with GlutaMAX™ I (cell culture media that contains L-Alanyl-L-Glutamine [a stabilized a form/source of L-Glutamine] from Invitrogen, Cat-No. 61870-010), 2.5% Fetal Calf Serum (FCS, Sigma Cat-No. F4135 (FBS)); 100 Units/ml penicillin/100 μg/ml streptomycin (=Pen/Strep from Invitrogen Cat. No. 15140). For the assay the cells were seeded in 384 well plates, 1000 cells per well, in the same medium. The next day the test compounds were added in various concentrations ranging from 30 μM to 0.0015 μM (10 concentrations, 1:3 diluted). After 5 days the viability assay was done according to the instructions of the manufacturer. In brief: the cell-plate was equilibrated to room temperature for approximately 30 minutes and than the reagent (containing luciferase, luciferan substrate, and buffer) was added. The contents were carefully mixed for 15 minutes to induce cell lysis. After 45 minutes the luminescent signal was measured in Victor 2, (scanning multiwell spectrophotometer, Wallac).

Details:

1st. Day:

Medium: RPMI 1640 with cell culture media containing L-Alanyl-L-Glutamine [GlutaMAX™ I (Invitrogen, Cat-Nr. 61870)], 5% FCS (Sigma Cat.-No. F4135), Pen/Strep (Invitrogen, Cat No. 15140).
HCT116 (ATCC-No. CCl-247): 1000 cells in 60 μl per well of 384 well plate (Greiner 781098, μClear-plate white)
After seeding incubate plates 24 h at 37° C., 5% CO₂
   2nd. Day: Induction (Treatment with Compounds, 10 Concentrations):

In order to achieve a final concentration of 30 μM as highest concentration 3.5 μL of 10 mM compound stock solution were added directly to 163 μl media. Then step e) of the dilution procedure described below, was followed.

In order to achieve the second highest to the lowest concentrations, a serial dilution with dilution steps of 1:3 was followed according to the procedure (a-e) as described here below:
a) for the second highest concentration add 10 μl of 10 mM stock solution of compound to 20 μl dimethylsulfoxide (DMSO)
b) dilute 8× 1:3 (always 10 μl to 20 μl DMSO) in this DMSO dilution row (results in 9 wells with concentrations from 3333.3 μM to 0.51 μM)
c) dilute each concentration 1:47.6 (3.5 μl compound dilution to 163 μl media)
d) add 10 μl of every concentration to 60 μl media in the cell plate resulting in final concentration of DMSO: 0.3% in every well and resulting in 10 final concentration of compounds ranging from 30 μM to 0.0015 μM.
Each compound is tested in triplicate.
Incubate 120 h (5 days) at 37° C., 5% CO₂
   Analysis:
Add 30 μl of reagent containing luciferase, luciferan substrate, and buffer (lyophilized) per well,
shake 15 minutes at room temperature
incubate further 45 minutes at room temperature without shaking
   Measurement:
Victor 2 scanning multiwell spectrophotometer (Wallac), Luminescence mode (0.5 sec/read, 477 nm)
Determine IC50 using a non-linear curve fit (XLfit® software [ID Business Solution Ltd., Guilford, Surrey, UK])

With all compounds a significant inhibition of HCT 116 cell viability was detected, which is exemplified by the compounds shown in Table 1.

TABLE 2
Results

| Examples | IC50 HCT 116 [μM] |
|---|---|
| 2 | 0.18 |
| 32 | 0.24 |
| 7 | 0.28 |
| 14 | 0.64 |
| 29 | 1.235 |
| 1, 3, 4, 5, 6, 8, 9, 20, 11, 13, 15, 17, | 0.1-1.0 |
| 19, 21, 22, 23, 25, 26, 28, 29, 34, 35, | |
| 37, 39, 40, 41, 43, 46, 47, 50, 51, 52, | |
| 55, 56, 58, 61, 65, 66, 69, 70, 72, 77, | |
| 79, 81, 85, 86, 88, 90, 95, 96, 101, | |
| 103, 108, 113, 116, 117, 122, 1, 24, | |
| 1, 27, 130, 132, 134, 137, 138, 140, | |
| 141, 142, 143, 144 | |
| 20, 36, 49, 59, 64, 82, 91, 100, 104, | 1.0-10 |
| 110, 128, 145 | |

The compounds according to this invention and their pharmaceutically acceptable salts or esters can be used as medicaments, e.g. in the form of pharmaceutical compositions. The pharmaceutical compositions can be administered orally, e.g. in the form of tablets, coated tablets, dragees, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically inert, inorganic or organic carriers. For example lactose, corn starch or derivatives thereof, talc, stearic acids or it's salts and the like can be used as carriers for tablets, coated tablets, dragées and hard gelatine capsules. Suitable carriers for soft gelatine capsules are, for example, vegetable oils, waxes, fats, semi-solid and liquid polyols and the like. However, depending on the nature of the active substance, carriers may not be required for some soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

A pharmaceutical compositions may comprise, for example, the following:

a) Tablet Formulation (Wet Granulation):

| Item | Ingredients | mg/tablet | | | |
|---|---|---|---|---|---|
| 1. | Compound of formula (I) | 5 | 25 | 100 | 500 |
| 2. | Lactose Anhydrous DTG | 125 | 105 | 30 | 150 |
| | (direct tabletting grade) | | | | |
| 3. | Sta-Rx 1500 (pre- | 6 | 6 | 6 | 30 |
| | gelatinized starch powder) | | | | |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure:
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

b) Capsule Formulation:

| Item | Ingredients | mg/capsule | | | |
|---|---|---|---|---|---|
| 1. | Compound of formula (I) | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure:
1. Mix items 1, 2 and 3 in a suitable mixer for 30 minutes.
2. Add items 4 and 5 and mix for 3 minutes.
3. Fill into a suitable capsule.
   c) Micro Suspension
1. Weigh 4.0 g glass beads in custom made tube GL 25, 4 cm (the beads fill half of the tube).
2. Add 50 mg compound, disperse with spatulum and vortex.
3. Add 2 ml gelatin solution (weight beads: gelatin solution=2:1) and vortex.
4. Cap and wrap in aluminum foil for light protection.
5. Prepare a counter balance for the mill.
6. Mill for 4 hours, 20/s in a Retsch mill (for some substances up to 24 hours at 30/s).
7. Extract suspension from beads with two layers of filter (100 μm) on a filter holder, coupled to a recipient vial by centrifugation at 400 g for 2 min.
8. Move extract to measuring cylinder.
9. Repeat washing with small volumes(here 1 ml steps) until final volume is reached or extract is clear.
10. Fill up to final volume with gelatin and homogenize.

The following examples and references are provided to aid the understanding of the present invention, the true scope of which is set forth in the appended claims. It is understood that modifications can be made in the procedures set forth without departing from the spirit of the invention.

### EXAMPLES

Experimental Procedures:

A: Starting Materials

### Preparation of 5,6-diamino-1-ethyl-3,3-dimethyl-1,3-dihydro-indol-2-one

### i) 1-Ethyl-3,3-dimethyl-6-nitro-1,3-dihydro-indol-2-one

A solution of 3,3-dimethyl-6-nitro-1,3-dihydro-indol-2-one (6 g, 29.10 mmol) in anhydrous N,N-dimethylformamide (DMF) (35 ml) was treated with sodium hydride. The resulting suspension was stirred for 1 h at 60° C. A solution of bromo-ethane (2.17 mL, 3.17 g, 29.10 mmol) in DMF (10 ml) was added. The mixture was allowed to cool to room temperature and stirred for 1 h. After removal of the solvent the mixture was quenched with water (100 ml) and extracted with ethyl acetate (3×100 ml). The extract was dried over Na₂SO₄, evaporated and the crude product was purified by column chromatography on silica gel. Elution with ethyl acetate/n-heptane (1:3) yielded 5.94 g (87%) of a yellow solid.

MS: M=235.3 (ESI+)

¹H-NMR (400 MHz, DMSO): δ (ppm)=1.16 (t, 3H), 1.32 (s, 6 H), 3.81 (q, 2H), 7.66 (d, 1H), 7.86 (s, 1H), 7.97 (d, 1H)

### ii) 6-Amino-1-ethyl-3,3-dimethyl-1,3-dihydro-indol-2-one

To a solution of 1-ethyl-3,3-dimethyl-6-nitro-1,3-dihydro-indol-2-one (5.9 g, 25.19 mmol) in methanol/tetrahydrofuran (THF) (1:1, 80 ml) palladium on charcoal (10%, 1.2 g) was added and the mixture hydrogenated at room temperature for 4 h. After filtration and evaporation of the solvents 5.05 g (98%) 6-amino-1-ethyl-3,3-dimethyl-1,3-dihydro-indol-2-one was isolated as white solid. MS: M=205.0 (API+)

¹H-NMR (400 MHz, DMSO): δ (ppm)=1.11 (t, 3H), 1.17 (s, 6H), 3.58 (q, 2H), 5.12 (br, 2H), 6.21 (d, 1H), 6.25 (s, 1H), 6.92 (d, 1H)

### iii) N-(1-Ethyl-3,3-dimethyl-2-oxo-2,3-dihydro-1H-indol-6-yl)-acetamide

A solution of 6-amino-1-ethyl-3,3-dimethyl-1,3-dihydro-indol-2-one (5.05 g, 24.72 mmol) in acetic anhydride (80 ml) was stirred at room temperature for 4 h. The mixture was poured onto ice water (150 ml), allowed to warm to room temperature and was stirred again for 2 h. After extraction with ethyl acetate (3×100 ml), the combined organic layers were washed with sat. NaHCO₃-solution (3×100 ml), brine (100 ml) and dried over sodium sulfate. After removal of the solvent the crude product was purified by column chromatography on silica gel (ethyl acetate/n-heptane 1:1) yielding 5.6 g (91%) N-(1-ethyl-3,3-dimethyl-2-oxo-2,3-dihydro-1H-indol-6-yl)-acetamide as light yellow solid.

MS: M=247.1 (API+)

¹H-NMR (400 MHz, DMSO): δ (ppm)=1.13 (t, 3H), 1.23 (s, 6H), 2.04 (s, 3H), 3.63 (q, 2H), 7.12 (d, 1 H), 7.23 (d, 1H), 7.37 (s, 1H), 9.97 (br, 1H)

### iv) N-(1-ethyl-3,3-dimethyl-5-nitro-2-oxo-2,3-dihydro-1H-indol-6-yl)-acetamide

To a solution of N-(1-ethyl-3,3-dimethyl-2-oxo-2,3-dihydro-1H-indol-6-yl)-acetamide (5.6 g, 22.73 mmol) in acetic anhydride (70 ml) nitric acid (100%, 1.96 g, 1.29 ml, 31.2 mmol) was added at 0° C. The mixture was stirred for 30 min, then poured onto ice water (150 ml). After stirring for 4 h the mixture was extracted with ethyl acetate (3×100 ml). The combined organic layers were washed with sodium hydroxide solution (1M, 100 ml) and water (100 ml), dried over sodium sulfate and concentrated. The crude product was purified by column chromatography on silica gel (ethyl acetate/n-heptane 1:1) to yield 5.2 g (78%) N-(1-ethyl-3,3-dimethyl-5-nitro-2-oxo-2,3-dihydro-1H-indol-6-yl)-acetamide as a yellow solid.

MS: M=292.0 (API+)

¹H-NMR (400 MHz, DMSO): δ (ppm)=1.16 (t, 3H), 1.31 (s, 6H), 2.13 (s, 3H), 3.71 (m, 2H), 7.54 (s, 1 H), 8.12 (s, 1H), 10.39 (br, 1H)

### v) 6-Amino-1-ethyl-3,3-dimethyl-5-nitro-1,3-dihydro-indol-2-one

N-(1-ethyl-3,3-dimethyl-5-nitro-2-oxo-2,3-dihydro-1H-indol-6-yl)-acetamide (5.2 g 17.85 mmol) was dissolved in ethanol (40 ml). After addition of hydrochloric acid (25%, 8 ml, 81.44 mmol) the mixture was stirred under reflux for 3 h. The reaction mixture was allowed to cool down to room temperature and then quenched with water (80 ml). The yellow precipitate was isolated by suction and washed with ethanol/water (1:1). The solid was dissolved in ethyl acetate, dried over sodium sulfate and concentrated to yield 4.15 g (93%) 6-amino-1-ethyl-3,3-dimethyl-5-nitro-1,3-dihydro-indol-2-one as a orange solid.

MS: M=250.0 (API+)

¹H-NMR (400 MHz, DMSO): δ (ppm)=1.15 (t, 3H), 1.27 (s, 6H), 3.64 (m, 2H), 6.54 (s, 1H), 7.67 (br, 2H), 7.95 (s, 1H)

### vi) 5,6-Diamino-1-ethyl-3,3-dimethyl-1,3-dihydro-indol-2-one

To a solution of 6-amino-1-ethyl-3,3-dimethyl-5-nitro-1,3-dihydro-indol-2-one (4.15 g, 16.65 mmol) in ethanol (80 ml) PtO₂ (0.4 g) was added and the mixture hydrogenated at room temperature for 3.5 h. After filtration and evaporation of the solvents 3.25 g (89%) 5,6-diamino-1-ethyl-3,3-dimethyl-1,3-dihydro-indol-2-one was isolated as orange solid.

MS: M=220.0 (API+)

¹H-NMR (400 MHz, DMSO): δ (ppm)=1.10 (t, 3H), 1.13 (s, 6H), 3.53 (m, 2H), 4.08 (br, 2H), 4.48 (br, 2H), 6.27 (s, 1H), 6.50 (s, 1H)

### Preparation of 5,6-diamino-1,3,3-trimethyl-1,3-dihydro-indol-2-one

5,6-diamino-1,3,3-trimethyl-1,3-dihydro-indol-2-one was prepared in an analogous 6-step-synthesis as described for 5,6-diamino-1-ethyl-3,3-dimethyl-1,3-dihydro-indol-2-one.

MS: M=206.1 (API+)

¹H-NMR (400 MHz, DMSO): δ (ppm)=1.57 (s, 6H), 3.43 (s, 3H), 4.94 (br, 4H), 6.66 (s, 1H), 6.95 (s, 1H)

### Preparation of 5,6-diamino-3,3-dimethyl-1-propyl-1,3-dihydro-indol-2-one

5,6-diamino-3,3-dimethyl-1-propyl-1,3-dihydro-indol-2-one was prepared in an analogous 6-step-synthesis as described for 5,6-diamino-1-ethyl-3,3-dimethyl-1,3-dihydro-indol-2-one.

MS: M=234.1 (API+)

¹H-NMR (400 MHz, DMSO): δ (ppm)=0.82 (t, 3H), 1.15 (s, 6H), 1.58 (m, 2H), 3.46 (q, 2H), 4.16 (br, 2H), 4.45 (br, 2H), 6.27 (s, 1H), 6.50 (s, 1H)

### Preparation of 5,6-diamino-1-isopropyl-3,3-dimethyl-1,3-dihydro-indol-2-one

5,6-diamino-3,3-dimethyl-1-isopropyl-1,3-dihydro-indol-2-one was prepared in an analogous 6-step-synthesis as described for 5,6-diamino-1-ethyl-3,3-dimethyl-1,3-dihydro-indol-2-one.

MS: M=234.1 (API+)

¹H-NMR (400 MHz, DMSO): δ (ppm)=1.12 (s, 6H), 1.33 (d, 6H), 4.09 (br, 2H), 4.40 (m, 1H), 4.46 (br, 2H), 6.46 (s, 1H), 6.48 (s, 1H)

### Preparation of 5,6-diamino-3,3-dimethyl-1-(3-morpholin-4-yl-propyl)-1,3-dihydro-indol-2-one

5,6-diamino-3,3-dimethyl-1-(3-morpholin-4-yl-propyl)-1,3-dihydro-indol-2-one was prepared in an analogous 6-step-synthesis as described for 5,6-diamino-1-ethyl-3,3-dimethyl-1,3-dihydro-indol-2-one.

MS: M=319.1 (API+)

¹H-NMR (400 MHz, DMSO): δ (ppm)=1.14 (s, 6H), 1.70 (m, 2H), 2.26 (t, 2H), 2.33 (m, 4H), 3.56 (m, 6H), 4.39 (br, 4H), 6.28 (s, 1H), 6.50 (s, 1H)

### Preparation of 1-allyl-5,6-diamino-3,3-dimethyl-1,3-dihydro-indol-2-one

A solution of 5,6-diamino-3,3-dimethyl-1,3-dihydro-indol-2-one (U.S. Pat. No. 4,666,923A) (1 g, 5.23 mmol) in anhydrous DMF (30 ml) was treated with sodium hydride (130 mg, 5.15 mmol) and stirred for 1 h at room temperature. 3-bromo-propene (450 μl, 629 mg, 5.20 mmol) was added dropwise. The resulting mixture was stirred for 4 h at room temperature and than poured into water (150 ml) and extracted with ethyl acetate (3×70 ml). The extract was dried over magnesium sulfate, evaporated carefully and under argon atmosphere and the crude product was purified by HPL chromatography. Yield 540 mg (45%) of a light yellow solid.

MS: M=232.4 (API+)

¹H-NMR (400 MHz, DMSO): δ (ppm)=1.22 (s, 6H), 4.19 (br, 4H), 4.54 (br, 2H), 5.08 (m, 1H), 5.18 (m, 1H), 5.87 (m, 1H), 6.24 (s, 1H), 6.57 (s, 1H)

### Preparation of 5,6-diamino-1-cyclopropylmethyl-3,3-dimethyl-1,3-dihydro-indol-2-one

5,6-diamino-1-cyclopropylmethyl-3,3-dimethyl-1,3-dihydro-indol-2-one was prepared in an analogous synthesis as described for 1-allyl-5,6-diamino-3,3-dimethyl-1,3-dihydro-indol-2-one.

MS: M=246.1 (API+)

¹H-NMR (400 MHz, DMSO): δ (ppm)=0.26 (m, 2H), 0.43 (m, 2H), 1.07 (m, 1H), 1.14 (s, 6H), 3.43 (d, 2H), 4.50 (br, 4H), 6.34 (s, 1H), 6.50 (s, 1H)

### Preparation of 1H-indazole-3-carbaldehyde

### i) (1H-indazol-3-yl)-methanol

1H-indazole-3-carboxylic acid (1 g, 6.17 mmol) was dissolved in diethyl ether (23 ml) the resulting solution was cooled to 0° C. Under an argon atmosphere and constant cooling a solution of lithium aluminium hydride (1 M in diethylether, 12.4 ml, 12.4 mmol) was added. The suspension was stirred at room temperature for 5 h and then quenched with sat. Na₂SO₄-solution (4 ml) and sat. NaHCO₃-solution (4 ml). After addition of ethyl acetate and stirring a jelly precipitate was formed. It was filtered and washed three times with ethyl acetate. The filtrate was concentrated to yield 645 mg (71%) of a light yellow solid.

MS: M=147.1 (ESI−)

¹H-NMR (400 MHz, DMSO): δ (ppm)=4.78 (d, 2H), 5.19 (t, 1H), 7.08 (t, 1H), 7.32 (t, 1H), 7.48 (d, 1H), 7.84 (d, 1H), 12.76 (br, 1H)

### ii) 1H-indazole-3-carbaldehyde

(1H-indazol-3-yl)-methanol (200 mg, 1.35 mmol) was dissolved in dichloromethane (10 ml). After addition of MnO2 (1.3 g, 13.46 mmol) it was stirred at room temperature for 16 h. The mixture was filtered and the filtrate was concentrated to yield 150 mg (76%) 1H-indazole-3-carbaldehyde.

MS: M=145.0 (API−)

¹H-NMR (400 MHz, DMSO): δ (ppm)=7.37 (t, 1H), 7.51 (t, 1H), 7.71 (d, 1H), 8.14 (d, 1H), 10.20 (s, 1H), 14.17 (br, 1H)

B: Final Products

### Example 1

### 5-Cyclopropylmethyl-2-(1H-indazol-3-yl)-7,7-dimethyl-5,7-dihydro-3H-imidazo[4,5-f]indol-6-one

5,6-Diamino-1-cyclopropylmethyl-3,3-dimethyl-1,3-dihydro-indol-2-one (190 mg, 0.82 mmol), 1H-indazole-3-carbaldehyde (80 mg, 0.82 mmol) and toluene-4-sulfonic acid monohydrate (10.5 mg, 0.05 mmol) were dissolved in ethanol (4 ml). Air was bubbled through the solution and it was stirred for 1 h under reflux. The mixture was concentrated and the crude product was purified by HPL chromatography. Yield 56 mg (29%) of a yellow solid.

MS: M=372.1 (API+)

¹H-NMR (400 MHz, DMSO): δ (ppm)=0.37 (m, 2H), 0.49 (m, 2H), 1.19 (m, 1H), 1.35 (s, 6H), 3.67 (m, 2H), 7.11 and 7.43 (s, 1H, two tautomeric forms), 7.30 (t, 1H), 7.45 (d, 1H), 7.49 and 7.73 (s, 1H, two tautomeric forms), 7.65 (d, 1H), 8.51 (t, 1H), 12.91 and 12.99 (br, 1H, two tautomeric forms), 13.54 and 13.58 (br, 1H, two tautomeric forms)

### Example 2

### 5-Allyl-2-(1H-indazol-3-yl)-7,7-dimethyl-5,7-dihydro-3H-imidazo[4,5-f]indol-6-one

In an analogous manner as described for example 1, 1-allyl-5,6-diamino-3,3-dimethyl-1,3-dihydro-indol-2-one was prepared from the appropriate starting material.

MS: M=358.0 (API+)

¹H-NMR (400 MHz, DMSO): δ (ppm)=1.38 (s, 6H), 4.39 (d, 2H), 5.18 (m, 2H), 5.91 (m, 1H), 6.96 and 7.27 (s, 1H, two tautomeric forms), 7.30 (t, 1H), 7.47 (t, 1H), 7.64 (d, 1H), 7.48 and 7.75 (s, 1H, two tautomeric forms), 8.49 (d, 1H), 12.90 and 13.00 (br, 1H, two tautomeric forms), 13.54 and 13.58 (br, 1H, two tautomeric forms)

### Example 3

### 5-Ethyl-2-(1H-indazol-3-yl)-7,7-dimethyl-5,7-dihydro-3H-imidazo[4,5-f]indol-6-one

5,6-Diamino-1-ethyl-3,3-dimethyl-1,3-dihydro-indol-2-one (400 mg, 1.82 mmol), and 1H-indazole-3-carboxylic acid (296 mg, 1.82 mmol) were mixed with polyphosphoric acid (6.08 g, 62.02 mmol) and phosphorus pentoxide (1.68 g, 11.86 mmol) and stirred under nitrogen at 150° C. for 6 h. It was quenched with ice water (50 ml) and the resulting suspension was adjusted to pH 7-8 by adding aqueous ammonia. The crude product was isolated by suction and washed with water. The solid was dried and purified by column chromatography on silica gel. Elution with ethyl acetate yielded 280 mg (97%) of 5-ethyl-2-(1H-indazol-3-yl)-7,7-dimethyl-5,7-dihydro-3H-imidazo[4,5-f]indol-6-one as a light yellow solid.

MS: M=346.1 (API+)

¹H-NMR (400 MHz, DMSO): δ (ppm)=1.21 (t, 3H), 1.34 (s, 6H), 3.78 (q, 2H), 7.04 and 7.39 (s, 1H, two tautomeric forms), 7.31 (t, 1H), 7.47 (t, 1H), 7.47 and 7.74 (s, 1H, two tautomeric forms), 7.65 (d, 1H), 8.51 (d, 1H), 12.96 (br, 1H), 13.58 (br, 1H)

### Example 4

### 2-(1H-indazol-3-yl)-5,7,7-trimethyl-5,7-dihydro-3H-imidazo[4,5-f]indol-6-one

In an analogous manner as described for example 3, 2-(1H-indazol-3-yl)-5,7,7-trimethyl-5,7-dihydro-3H-imidazo[4,5-f]indol-6-one was prepared from the appropriate starting material.

MS: M=332.1 (API+)

¹H-NMR (400 MHz, DMSO): δ (ppm)=1.35 (s, 6H), 3.22 (s, 3H), 7.02 and 7.34 (s, 1H, two tautomeric forms), 7.30 (t, 1H), 7.48 (d, 1H), 7.47 and 7.72 (s, 1H, two tautomeric forms), 7.64 (d, 1H), 8.50 (t, 1H), 12.96 and 13.00 (br, 1H, two tautomeric forms), 13.55 and 13.59 (br, 1H, two tautomeric forms)

### Example 5

### 2-(1H-indazol-3-yl)-7,7-dimethyl-5-propyl-5,7-dihydro-3H-imidazo[4,5-f]indol-6-one

In an analogous manner as described for example 3, 2-(1H-indazol-3-yl)-7,7-dimethyl-5-propyl-5,7-dihydro-3H-imidazo[4,5-f]indol-6-one was prepared from the appropriate starting material.

MS: M=360.1 (API+)

¹H-NMR (400 MHz, DMSO): δ (ppm)=0.89 (t, 3H), 1.34 (s, 6H), 1.68 (m, 2H), 3.73 (m, 2H), 7.02 and 7.38 (s, 1H, two tautomeric forms), 7.29 (t, 1H), 7.46 (t, 1H), 7.63 (d, 1H), 7.44 and 7.73 (s, 1H, two tautomeric forms), 8.51 (d, 1H), 12.90 and 12.99 (br, 1H), 13.53 and 13.56 (br, 1H, two tautomeric forms)

### Example 6

### 2-(1H-indazol-3-yl)-5-isopropyl-7,7-dimethyl-5,7-dihydro-3H-imidazo[4,5-f]indol-6-one

In an analogous manner as described for example 3, 2-(1H-indazol-3-yl)-5-isopropyl-7,7-dimethyl-5,7-dihydro-3H-imidazo[4,5-f]indol-6-one was prepared from the appropriate starting material.

MS: M=360.2 (API+)

¹H-NMR (400 MHz, DMSO): δ (ppm)=1.32 (s, 6H), 1.48 (d, 6H), 4.56 (m, 1H), 7.14 and 7.43 (s, 1H, two tautomeric forms), 7.29 (t, 1H), 7.45 (d, 1H), 7.48 and 7.71 (s, 1H, two tautomeric forms), 7.63 (d, 1H), 8.50 (t, 1H), 12.84 and 12.97 (br, 1H), 13.54 and 13.57 (br, 1H)

### Example 7

### 2-(1H-indazol-3-yl)-7,7-dimethyl-5-(3-morpholin-4-yl-propyl)-5,7-dihydro-3H-imidazo[4,5-f]indol-6-one

In an analogous manner as described for example 3, 2-(1H-indazol-3-yl)-7,7-dimethyl-5-(3-morpholin-4-yl-propyl)-5,7-dihydro-3H-imidazo[4,5-f]indol-6-one was prepared from the appropriate starting material.

MS: M=445.2 (API+)

¹H-NMR (400 MHz, DMSO): δ (ppm)=1.34 (s, 6H), 1.81 (m, 2H), 2.33 (m, 6H), 3.60 (m, 4H), 3.78 (m, 2H), 7.09 and 7.43 (s, 1H, two tautomeric forms), 7.30 (t, 1H), 7.47 (t, 1H), 7.49 and 7.72 (s, 1H, two tautomeric forms), 7.64 (d, 1H), 8.50 (d, 1H), 12.97 and 12.99 (br, 1H, two tautomeric forms), 13.56 (br, 1H)

### Example 8

### 2-(1H-indazol-3-yl)-7,7-dimethyl-5,7-dihydro-3H-imidazo[4,5-f]indol-6-one

In an analogous manner as described for example 3, 2-(1H-indazol-3-yl)-7,7-dimethyl-5,7-dihydro-3H-imidazo[4,5-f]indol-6-one was prepared from the appropriate starting material.

MS: M=318.1 (API+)

¹H-NMR (400 MHz, DMSO): δ (ppm)=1.32 (s, 6H), 6.94 and 7.10 (s, 1H, two tautomeric forms), 7.29 (t, 1H), 7.47 (t, 1H), 7.38 and 7.64 (s, 1H, two tautomeric forms), 7.63 (d, 1H), 8.49 (d, 1H), 10.30 (br, 1H), 12.77 and 12.92 (br, 1H, two tautomeric forms), 13.55 (br, 1H)

### Example 9

### 2-(1H-indazol-3-yl)-spiro[7,7-cyclopentan-5,7-dihydro-3H-imidazo[4,5-f]indol]-6-one (or according to the actual IUPAC-nomenclature:2-(1H-Indazol-3-yl)-spiro-5,7-dihydro[cyclopentane-1′,7-imidazo[4,5-f]indol]-6(3H)-one)

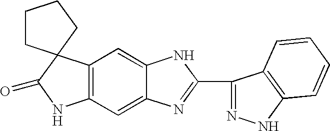

In an analogous manner as described for example 3, 2-(1H-indazol-3-yl)-spiro[7,7-cyclopentan-5,7-dihydro-3H-imidazo[4,5-f]indol]-6-one was prepared from the appropriate starting material.

MS: M=344.0 (API+)

### Example 10

### 2-(1H-Indazol-3-yl)-8,8-dimethyl-1,5,7,8-tetrahydro-imidazo[4,5-g]quinolin-6-one

In an analogous manner as described for example 3, 2-(1H-Indazol-3-yl)-8,8-dimethyl-1,5,7,8-tetrahydro-imidazo[4,5-g]quinolin-6-one was prepared from the appropriate starting material.

MS: M=332.4 (ES+)

¹H-NMR (400 MHz, DMSO): δ (ppm)=1.31 (s, 3H), 1.32 (s, 3H), 2.38 (s, 2H), 7.07 and 7.23 (s, 1H, two tautomeric forms), 7.30 (t, 1H), 7.38 and 7.64 (s, 1H, two tautomeric forms), 7.47 (t, 1H), 7.64 (m, 1H), 8.49 (d, 1H), 10.15 and 10.20 (br, 1H, two tautomeric forms), 12.76 and 12.83 (br, 1H, two tautomeric forms), 13.57 and 13.60 (br, 1H, two tautomeric forms)

### Example 11

### 2-(1H-Indazol-3-yl)-7-methyl-5,7-dihydro-3H-imidazo[4,5-f]indol-6-one

In an analogous manner as described for example 3, 2-(1H-indazol-3-yl)-7-methyl-5,7-dihydro-3H-imidazo[4,5-f]indol-6-one was prepared from 5,6-diamino-3-methyl-1,3-dihydro-indol-2-one (DE3417643A1) and 1H-indazole-3-carboxylic acid.

MS: M=304.1 (API+)

¹H-NMR (400 MHz, DMSO): δ (ppm)=1.39 (d, 3H), 3.47 (m, 1H), 6.92 and 7.08 (s, 1H, two tautomeric forms), 7.29 (t, 1H), 7.37 and 7.60 (s, 1H), two tautomeric forms), 7.46 (t, 1H), 7.62 (d, 1H), 8.48 (d, 1H), 10.28 and 10.33 (br, 1H, two tautomeric forms), 12.77 and 12.88 (br, 1H, two tautomeric forms), 13.53 (br, 1H)

### Example 12

### 5,7,7-Triethyl-2-(1H-indazol-3-yl)-5,7-dihydro-3H-imidazo[4,5-f]indol-6-one

To a solution of 1H-indazole-3-carboxylic acid (100 mg, 0.617 mmol), 1-hydroxybenzotriazole hydrate (113.3 mg, 0.740 mmol) and triethylamine (187.2 mg, 1.85 mmol) in DMF (4 ml) was added N′-(3-dimethylaminopropyl)-N-ethylcarbodiimide hydrochloride (141.9 mg, 0.740 mmol). After 30 minutes at room temperature a solution of 5,6-diamino-1,3,3-triethyl-1,3-dihydro-indol-2-one (152.5 mg, 0.617 mmol) in DMF (2 ml) was added and stirring continued for further 20 minutes. The solvent was evaporated under reduced pressure and the residue was dissolved in water. The aqueous phase was extracted twice with ethyl acetate and the solvent of the combined organic phases was evaporated yielding 346 mg of an oil that was used without further purification. The oil was dissolved in ethanol (7 ml), treated with aqueous HCl solution (32%, 4 ml) and heated under reflux for 2 h. The solvent was evaporated, the residue alkalized with aqueous ammonia (25%). The aqueous phase was extracted three times with ethyl acetate and the combined organic phases were washed with brine, dried over MgSO₄ and evaporated. The residue was subjected to silica gel chromatography (ethyl acetate/heptane 1:1→2:1→9:1) to yield 126 mg 5,7,7-triethyl-2-(1H-indazol-3-yl)-5,7-dihydro-3H-imidazo[4,5-f]indol-6-one (0.337 mmol, 55%).

MS: M=374.1 (ESI+)

¹H-NMR (400 MHz, DMSO): δ (ppm)=0.48 (t, 6H), 1.29 (t, 3H), 1.82 (m, 4H), 3.80 (t, 2H), 7.03 and 7.34 (s, 1H, two tautomeric forms), 7.30 (t, 1H), 7.38 and 7.63 (s, 1H, two tautomeric forms), 7.47 (t, 1H), 7.64 (d, 1H), 8.50 (d, 1H), 12.93 (br, 1H), 13.54 (br, 1H)

### Example 13

### 7-Ethyl-2-(1H-indazol-3-yl)-5,7-dihydro-3H-imidazo[4,5-f]indol-6-one

In an analogous manner as described for example 12, 7-ethyl-2-(1H-indazol-3-yl)-5,7-dihydro-3H-imidazo[4,5-f]indol-6-one was prepared from 5,6-diamino-3-ethyl-1,3-dihydro-indol-2-one (DE3417643A1) and 1H-indazole-3-carboxylic acid.

MS: M=318.0 (ESI+)

¹H-NMR (400 MHz, DMSO): δ (ppm)=0.81 (t, 3H), 1.87-1.99 (m, 2H), 3.48 (m, 1H), 6.92 and 7.08 (s, 1H, two tautomeric forms), 7.29 (t, 1H), 7.37 and 7.59 (s, 1H, two tautomeric forms), 7.46 (t, 1H), 7.63 (d, 1H), 8.49 (d, 1H), 10.29 and 10.34 (br, 1H, two tautomeric forms), 12.77 and 1287 (br, 1H, two tautomeric forms), 13.53 (br, 1H)

### Example 14

### 5-Ethyl-2-(1H-indazol-3-yl)-8,8-dimethyl-3,5,7,8-tetrahydro-imidazo[4,5-g]quinolin-6-one

A mixture of 6,7-diamino-1-ethyl-4,4-dimethyl-3,4-dihydro-1H-quinolin-2-one (70 mg, 0.300 mmol), 1H-indazole-3-carbaldehyde (44 mg, 0.301 mmol) and sulfur (10.5 mg, 0.327 mmol) in DMF (3 ml) was heated at 155° C. for 30 minutes. After cooling to room temperature the reaction mixture was treated with water. After stirring for 30 minutes the precipitate was filtered off and washed with water to yield 94 mg 5-ethyl-2-(1H-indazol-3-yl)-8,8-dimethyl-3,5,7,8-tetrahydro-imidazo[4,5-g]quinolin-6-one (87%).

MS: M=360.3 (ESI+)

¹H-NMR (400 MHz, DMSO): δ (ppm)=1.20 (bt, 3H), 1.30 (s, 6H), 2.46 (s, 2H), 4.08 (bq, 2H), 7.07-7.82 (m, 2H), 7.30 (t, 1H), 7.47 (t, 1H), 7.65 (d, 1H), 8.51 (d, 1H), 12.81 and 12.89 (bs, 1H), 13.59 (s, 1H)

### Example 15

### 5-But-3-enyl-2-(1H-indazol-3-yl)-7,7-dimethyl-5,7-dihydro-3H-imidazo[4,5-f]indol-6-one

In an analogous manner as described for example 14, 5-but-3-enyl-2-(1H-indazol-3-yl)-7,7-dimethyl-5,7-dihydro-3H-imidazo[4,5-f]indol-6-one was prepared from 5,6-diamino-1-but-3-enyl-3,3-dimethyl-1,3-dihydro-indol-2-one and 1H-indazole-3-carbaldehyde. 5,6-Diamino-1-but-3-enyl-3,3-dimethyl-1,3-dihydro-indol-2-one was prepared in an analogous manner as described for 1-allyl-5,6-diamino-3,3-dimethyl-1,3-dihydro-indol-2-one (see part A, starting materials) using 4-bromo-1-butene instead of 3-bromo-propene as alkylating agent.

MS: M=372.1 (API+)

¹H-NMR (400 MHz, DMSO): δ (ppm)=1.33 (s, 6H), 2.44 (m, 2H), 3.83 (t, 2H), 4.91-5.07 (m, 2H), 5.82 (m, 1H), 7.04-7.70 (m, 2H), 7.30 (t, 1H), 7.47 (t, 1H), 7.64 (d, 1H), 8.50 (d, 1H), 12.85-13.09 (bs, 1H), 13.56 (bs, 1H)

In an analogous manner as described for example 15 the following examples 16-25 were prepared from the appropriate starting materials:

| Example | | ¹H-NMR(400MHz, | |
|---|---|---|---|
| No | Systematic Name | DMSO): δ(ppm)= | MS: M= |
| 16 | 2-(1H-Indazol-3-yl)-7,7- | 1.42(s, 6H), 5.06(s, 2H), | 409.1(API+) |
| | dimethyl-5-pyridin-3- | 6.94 and 7.27(s, 1H, two | |
| | ylmethyl-5,7-dihydro- | tautomeric forms), | |
| | 3H-imidazo[4,5-f]indol- | 7.28-7.77(m, 6h), 8.46-8.49(m, | |
| | 6-one | 2H), 8.62 and 8.67(s, 1H, | |
| | | two tautomeric forms), | |
| | | 12.84 and 13.01(s, 1H, two | |
| | | tautomeric forms), 13.55(s, | |
| | | 1H) | |
| 17 | 2-(1H-Indazol-3-yl)-5- | 1.34(s, 6H), 3.19(s, 3H), | 420.2(ESI+) |
| | [2-(2-methoxy-ethoxy)- | 3.38-3.51(m, 2H), 3.54(t, | |
| | ethyl]-7,7-dimethyl-5,7- | 2H), 3.69(t, 2H), 3.93(t, | |
| | dihydro-3H- | 2H), 7.12-7.49(m, 4H), | |
| | imidazo[4,5-f]indol-6- | 7.64 and 7.66(s, 1H, two | |
| | one | tautomeric forms), 8.49 and | |
| | | 8.51(s, 1H, two tautomeric | |
| | | forms), 12.96(s, 1H), | |
| | | 13.56(s, 1H) | |
| 18 | 2-(1H-Indazol-3-yl)-5- | 1.34(s, 6H), 3.27(s, 3H), | 376.1(API+) |
| | (2-methoxy-ethyl)-7,7- | 3.62(q, 2H), 3.93(q, 2H), | |
| | dimethyl-5,7-dihydro- | 7.10 and 7.72(s, 1H, two | |
| | 3H-imidazo[4,5-f]indol- | tautomeric forms), 7.30(t, | |
| | 6-one | 1H), 7.40-7.49(m, 2H), | |
| | | 7.65(d, 1H), 8.51(t, 1H), | |
| | | 12.91 and 12.98(s, 1H, two | |
| | | tautomeric forms), 13.54 | |
| | | and 13.58(s, 1H, two | |
| | | tautomeric forms) | |
| 19 | 2-(1H-Indazol-3-yl)-7,7- | 1.34(s, 6H), 1.41(m, 2H), | 443.1(API+) |
| | dimethyl-5-(3- | 1.55(m, 4H), 1.85(m, 2H), | |
| | piperidin-1-yl-propyl)- | 2.44(m, 3H), 3.78(m, 2H), | |
| | 5,7-dihydro-3H- | 7.07 and 7.43(s, 1H), | |
| | imidazo[4,5-f]indol-6- | 7.30(t, 1H), 7.44 and 7.72(s, | |
| | one | 1H), 7.46(t, 1H), 7.64(m, | |
| | | 1H), 8.50(m, 1H), 12.95 | |
| | | and 12.99(s, 1H), 13.54 and | |
| | | 13.58(s, 1H) | |
| 20 | 5-(2-Diisopropylamino- | (400MHz, CDCl3) 1.00(d, | 445.2(API+) |
| | ethyl)-2-(1H-indazol-3- | 12H), 1.49(s, 6H), 2.96(t, | |
| | yl)-7,7-dimethyl-5,7- | 2H), 3.12(m, 2H), 4.75(m, | |
| | dihydro-3H- | 2H), 7.34(m, 3H), 7.48(m, | |
| | imidazo[4,5-f]indol-6- | 1H), 7.56(d, 1H), 7.85(s, | |
| | one | 1H), 8.63(d, 1H) | |
| 21 | 5-(3-Dimethylamino- | 1.34(s, 6H), 1.76-1.81(m, | 403.1(API+) |
| | propyl)-2-(1H-indazol- | 2H), 2.17(s, 6H), | |
| | 3-yl)-7,7-dimethyl-5,7- | 2.27-2.32(m, 2H), 3.77(t, 2H), 7.09 | |
| | dihydro-3H- | and 7.39(s, 1H, two | |
| | imidazo[4,5-f]indol-6- | tautomeric forms), 7.30(t, | |
| | one | 1H), 7.46(d, 1H), 7.49 and | |
| | | 7.72(s, 1H, two tautomeric | |
| | | forms), 7.64(d, 1H), 8.50(t, | |
| | | 1H), 12.92 and 12.99(s, 1H, | |
| | | two tautomeric forms), | |
| | | 13.54 and 13.58(s, 1H, two | |
| | | tautomeric forms) | |
| 22 | 5-(2-Diethylamino- | 0.91(t, 6H), 1.34(s, 6H), | 417.2(API+) |
| | ethyl)-2-(1H-indazol-3- | 2.67(m, 2H), 3.22(t, 4H), | |
| | yl)-7,7-dimethyl-5,7- | 3.37(t, 2H), 4.11(t, 2H), | |
| | dihydro-3H- | 7.06 and 7.34(s, 1H, two | |
| | imidazo[4,5-f]indol-6- | tautomeric forms), 7.30(t, | |
| | one | 1H), 7.43-7.47(m, 1H), | |
| | | 7.49 and 7.70(s, 1H, two | |
| | | tautomeric forms), 7.64(d, | |
| | | 1H), 8.50(d, 1H), 12.90 and | |
| | | 12.98(s, 1H, two tautomeric | |
| | | forms), 13.54(s, 1H) | |
| 23 | [2-(1H-Indazol-3-yl)- | 1.21(t, 3H), 1.37(s, 6H), | 404.0(API+) |
| | 7,7-dimethyl-6-oxo-6,7- | 4.16(q, 2H), 4.63(s, 2H), | |
| | dihydro-3H- | 7.01 and 7.33(s, 1H), | |
| | imidazo[4,5-f]indol-5- | 7.30(t, 1H), 7.47 and 7.75(s, | |
| | yl]-acetic acid ethyl ester | 1H), 7.47(t, 1H), 7.64(d, | |
| | | 1H), 8.49(d, 1H), 12.95 and | |
| | | 13.01(s, 1H), 13.54 and | |
| | | 13.58(s, 1H) | |
| 24 | [2-(1H-Indazol-3-yl)- | 1.39(s, 6H), 5.04(d, 2H), | 357.1(API+) |
| | 7,7-dimethyl-6-oxo-6,7- | 7.28 and 7.52(s, 1H), | |
| | dihydro-3H- | 7.30(t, 1H), 7.47(t, 1H), 7.58 | |
| | imidazo[4,5-f]indol-5- | and 7.81(s, 1H), 7.65(d, | |
| | yl]-acetonitrile | 1H), 8.51(m, 1H), 13.11(s, | |
| | | 1H), 13.58 and 13.61(s, 1H) | |
| 25 | 2-(1H-Indazol-3-yl)-7,7- | 1.36(s, 6H), 2.14(s, 3H), | 392.3(ESI+) |
| | dimethyl-5-(2- | 2.83(m, 2H), 3.98(t, 2H), | |
| | methylsulfanyl-ethyl)- | 7.08 and 7.74(s, 1H, two | |
| | 5,7-dihydro-3H- | tautomeric forms), 7.30(t, | |
| | imidazo[4,5-f]indol-6- | 1H), 7.46(m, 2H), 7.65(d, | |
| | one | 1H), 8.50(s, 1H), 12.93 and | |
| | | 13.01(s, 1H, two tautomeric | |
| | | forms), 13.57(s, 1H) | |

### Example 26

### 5-Benzyl-2-(1H-indazol-3-yl)-7,7-dimethyl-5,7-dihydro-3H-imidazo[4,5-f]indol-6-one

In an analogous manner as described for example 1, 5-benzyl-2-(1H-indazol-3-yl)-7,7-dimethyl-5,7-dihydro-3H-imidazo[4,5-f]indol-6-one was prepared from 5,6-diamino-1-benzyl-3,3-dimethyl-1,3-dihydro-indol-2-one and 1H-indazole-3-carbaldehyde. 5,6-Diamino-1-benzyl-3,3-dimethyl-1,3-dihydro-indol-2-one was prepared in an analogous manner as described for 1-allyl-5,6-diamino-3,3-dimethyl-1,3-dihydro-indol-2-one (see part A, starting materials) using benzyl bromide instead of 3-bromo-propene as alkylating agent.

MS: M=408.0 (API+)

¹H-NMR (400 MHz, DMSO): δ (ppm)=1.43 (d, 6H), 5.00 (s, 2H), 6.88 and 7.17 (s,1H, two tautomeric forms), 7.26-7.45 (m, 7H), 7.47 and 7.77 (s, 1H, two tautomeric forms), 7.63 (d, 1H), 8.46 (m, 1H), 12.80 and 13.99 (s, 1H), 13,54 (d, 1H)

In an analogous manner as described for example 26 the following examples 27-29 were prepared from the appropriate starting materials:

| Example | | ¹H-NMR(400MHz, | |
|---|---|---|---|
| No | Systematic Name | DMSO): δ(ppm)= | MS: M= |
| 27 | 2-(1H-Indazol-3-yl)-5- | 1.43(s, 3H), 1.45(s, 3H), | 486.2(ESI+) |
| | (4-methanesulfonyl-benzyl)- | 3.18(m, 3H), 5.13(m, 2H), | |
| | 7,7-dimethyl- | 6.88 and 7.23(s, 1H), | |
| | 5,7-dihydro-3H- | 7.28(m, 1H), 7.45(t, 1H), 7.49 | |
| | imidazol[4,5-f]indol-6- | and 7.79(s, 1H), 7.54(d, | |
| | one | 1H), 7.62(m, 2H), 7.92(m, | |
| | | 2H), 8.45(m, 1H), 12.82 | |
| | | and 13.02(s, 1H), 13.50 and | |
| | | 13.57(s, 1H) | |
| 28 | 5-[2-(tert-Butyl-dimethyl- | −0.07(s, 6H), 0.76(m, 9H), | 476.3(ESI+) |
| | silanyloxy)- | 1.34(m, 6H), 3.87(m, 4H), | |
| | ethyl]-2-(1H-indazol-3- | 7.08 and 7.37(s, 1H), | |
| | yl)-7,7-dimethyl-5,7- | 7.29(m, 1H), 7.42 and 7.69(s, | |
| | dihydro-3H- | 1H), 7.46(t, 1H), 7.64(d, | |
| | imidazo[4,5-f]indol-6- | 1H), 8.49(d, 1H), 12.92 and | |
| | one | 12.96(s, 1H), 13.53 and | |
| | | 13.57(s, 1H) | |
| 29 | 5-(2-Hydroxy-3- | 1.34(s, 6H), 2.40(m, 6H), | 461.5(ESI+) |
| | morpholin-4-yl-propyl)- | 3.51-3.71(m, 5H), | |
| | 2-(1H-indazol-3-yl)-7,7- | 3.85(m, 1H), 4.08(m, 1H), | |
| | dimethyl-5,7-dihydro- | 4.95(m, 1H), 7.14 and 7.42(s, | |
| | 3H-imidazo[4,5-f]indol- | 1H), 7.29(t, 1H), 7.44 and | |
| | 6-one | 7.70(s, 1H), 7.46(t, 1H), | |
| | | 7.64(d, 1H), 8.50(m, 1H), | |
| | | 11.94(bs, 1H), 12.94 and | |
| | | 12.97(s, 1H) | |

### Example 30

### 5-(Dimethyl-phosphinoylmethyl)-2-(1H-indazol-3-yl)-7,7-dimethyl-5,7-dihydro-3H-imidazo[4,5-f]indol-6-one

In an analogous manner as described for example 3, 5-(dimethyl-phosphinoylmethyl)-2-(1H-indazol-3-yl)-7,7-dimethyl-5,7-dihydro-3H-imidazo[4,5-f]indol-6-one was prepared from 5,6-diamino-1-(dimethyl-phosphinoylmethyl)-3,3-dimethyl-1,3-dihydro-indol-2-one and 1H-indazole-3-carboxylic acid. 5,6-Diamino-1-(dimethyl-phosphinoylmethyl)-3,3-dimethyl-1,3-dihydro-indol-2-one was prepared in an analogous manner as described for 1-allyl-5,6-diamino-3,3-dimethyl-1,3-dihydro-indol-2-one (see part A, starting materials) using chloromethyl(dimethyl)phosphine oxide instead of 3-bromo-propene as alkylating agent.

MS: M=408.0 (API+)

¹H-NMR (400 MHz, DMSO): δ (ppm)=1.38 (m, 6H), 1.47 (s, 3H), 1.50 (s, 3H), 4.22 (m, 2H), 7.29 (t, 1H), 7.31 and 7.44 (s, 1H), 7.46 (t, 1H), 7.58 and 7.73 (s, 1H), 7.64 (m, 1H), 8.50 (m, 1H), 12.97 and 13.00 (s, 1H), 13.53 and 13.58 (s, 1H)

### Example 31

### 2-[2-(1H-Indazol-3-yl)-7,7-dimethyl-6-oxo-6,7-dihydro-3H-imidazo[4,5-f]indol-5-yl]-thioacetamide

2-[2-(1H-Indazol-3-yl)-7,7-dimethyl-6-oxo-6,7-dihydro-3H-imidazo[4,5-f]indol-5-yl]-thioacetamide was obtained as a byproduct in the synthesis of [2-(1H-indazol-3-yl)-7,7-dimethyl-6-oxo-6,7-dihydro-3H-imidazo[4,5-f]indol-5-yl]-acetonitrile (example 24).

MS: M=391.0 (API+)

¹H-NMR (400 MHz, DMSO): δ (ppm)=1.40 (bs, 6H), 4.62 (bs, 2H), 6.85 and 7.09 (s, 1H), 7.29 (t, 1H), 7.44 and 7.72 (s, 1H), 7.46 (t, 1H), 7.64 (d, 1H), 8.50 (d, 1H), 9.41 and 9.82 (s, 2H), 12.89 and 12.98 (s, 1H), 13.52 and 13.57 (s, 1H)

### Example 32

### 2-(1H-Indazol-3-yl)-7,7-dimethyl-5-thiazol-2-ylmethyl-5,7-dihydro-3H-imidazo[4,5-f]indol-6-one

A solution of 2-[2-(1H-indazol-3-yl)-7,7-dimethyl-6-oxo-6,7-dihydro-3H-imidazo[4,5-f]indol-5-yl]-thioacetamide (example 31, 25 mg, 0.064 mmol) and 1,2-dichloro-diethylether (11 mg, 0.066 mmol) in DMF (0.3 ml) was heated to 140° C. for 3.5 h. Purification by HPLC chromatography yielded 12.8 mg 2-(1H-indazol-3-yl)-7,7-dimethyl-5-thiazol-2-ylmethyl-5,7-dihydro-3H-imidazo[4,5-f]indol-6-one (0.031 mmol, 48%).

MS: M=413.0 (API−)

¹H-NMR (400 MHz, DMSO): δ (ppm)=1.41 (s, 6H), 5.32 (s, 2H), 7.06 and 7.28 (s, 1H), 7.28 (t, 1H), 7.45 and 7.77 (s, 1H), 7.46 (t, 1H), 7.63 (d, 1H), 7.68 (d, 1H), 7.79 (d, 1H), 8.47 (d, 1H), 12.89 and 13.03 (s, 1H), 13.55 (s, 1H)

### Example 33

### 2-(1H-Indazol-3-yl)-7,7-dimethyl-5-(1H-tetrazol-5-ylmethyl)-5,7-dihydro-3H-imidazo[4,5-f]indol-6-one

A solution of [2-(1H-indazol-3-yl)-7,7-dimethyl-6-oxo-6,7-dihydro-3H-imidazo[4,5-f]indol-5-yl]-acetonitrile (example 24, 50 mg, 0.140 mmol), sodium azide (109.5 mg, 1.68 mmol) and ammonium chloride (91 mg, 1.701 mmol) in DMF (1.5 ml) in a sealed tube was heated in a microwave at 15 Watt for 40 minutes. During that time temperature reached 225° C. and pressure 14 bar. After cooling to room temperature the reaction mixture was added to saturated NaHCO₃ solution (35 ml). The aqueous phase was washed twice with ethyl acetate and then acidified with concentrated hydrochloric acid to pH1. The aqueous phase was extracted with n-butanol, the organic phase was dried and the solvent evaporated. The residue was triturated with diisopropyl ether and ethyl acetate and then purified by HPLC chromatography to yield 18.9 mg 2-(1H-indazol-3-yl)-7,7-dimethyl-5-(1H-tetrazol-5-ylmethyl)-5,7-dihydro-3H-imidazo[4,5-f]indol-6-one (0.047 mmol, 34%).

MS: M=398.0 (API−)

¹H-NMR (400 MHz, DMSO): δ (ppm)=1.40 (s, 6H), 5.33 (s, 2H), 6.96-7.82 (m, 2H), 7.29 (t, 1H), 7.46 (t, 1H), 7.63 (d, 1H), 8.48 (d, 1H), 12.91 and 13.03 (s, 1H), 13.55 (s, 1H),

### Example 34

### 5-(2-Hydroxy-ethyl)-2-(1H-indazol-3-yl)-7,7-dimethyl-5,7-dihydro-3H-imidazo[4,5-f]indol-6-one

To a solution of 5-[2-(tert-butyl-dimethyl-silanyloxy)-ethyl]-2-(1H-indazol-3-yl)-7,7-dimethyl-5,7-dihydro-3H-imidazo[4,5-f]indol-6-one (example 28, 80 mg, 0.168 mmol) in THF (2 ml) was added a solution of tetrabutylammonium fluoride (1M, 505 μl, 0.505 mmol). After 1 h at room temperature the reaction mixture was concentrated and the residue treated with water. The resulting precipitate was filtered off: 25 mg 5-(2-hydroxy-ethyl)-2-(1H-indazol-3-yl)-7,7-dimethyl-5,7-dihydro-3H-imidazo[4,5-f]indol-6-one (0.069 mmol, 41%).

MS: M=362.3 (ESI+)

¹H-NMR (400 MHz, DMSO): δ (ppm)=1.34 (s, 6H), 3.65 (t, 2H), 3.81 (t, 2H), 4.90 (bs, 1H), 7.13-7.76 (m, 2H), 7.29 (t, 1H), 7.46 (t, 1H), 7.64 (d, 1H), 8.50 (d, 1H)

### Example 35

### 5-(2,3-Dihydroxy-propyl)-2-(1H-indazol-3-yl)-7,7-dimethyl-5,7-dihydro-3H-imidazo [4,5-f]indol-6-one

In an analogous manner as described for example 1, 5-(2,3-Dihydroxy-propyl)-2-(1H-indazol-3-yl)-7,7-dimethyl-5,7-dihydro-3H-imidazo[4,5-f]indol-6-one was prepared from 5,6-diamino-1-(2,2-dimethyl-[1,3]dioxolan-4-ylmethyl)-3,3-dimethyl-1,3-dihydro-indol-2-one and 1H-indazole-3-carbaldehyde. 5,6-Diamino-1-(2,2-dimethyl-[1,3]dioxolan-4-ylmethyl)-3,3-dimethyl-1,3-dihydro-indol-2-one was prepared in an analogous manner as described for 1-allyl-5,6-diamino-3,3-dimethyl-1,3-dihydro-indol-2-one (see part A, starting materials) using 4-bromomethyl-2,2-dimethyl-[1,3]dioxolane instead of 3-bromo-propene as alkylating agent.

MS: M=392.0 (API+)

¹H-NMR (400 MHz, DMSO): δ (ppm)=1.35 (s, 6H), 3.39 (bd, 2H), 3.58-3.95 (m, 3H), 4.68 (bs, 1H), 4.95 (bs, 1H), 7.15 and 7.42 (s, 1H), 7.29 (t, 1H), 7.46 and 7.70 (s, 1H), 7.46 (t, 1H), 7.64 (d, 1H), 8.50 (m. 1H), 12.90 and 12.96 (s, 1H), 13.52 and 13.57 (s, 1H)

### Example 36

### [2-(1H-Indazol-3-yl)-7,7-dimethyl-6-oxo-6,7-dihydro-3H-imidazo[4,5-f]indol-5-yl]-acetic acid

A solution of [2-(1H-indazol-3-yl)-7,7-dimethyl-6-oxo-6,7-dihydro-3H-imidazo[4,5-f]indol-5-yl]-acetic acid ethyl ester (example 23, 50 mg, 0.124 mmol) in THF (4 ml) was treated with lithium hydroxide (6 mg, 0.250 mmol) and heated to 70° C. After 2 and 3.5 h further four and two equivalents lithium hydroxide were added to the reaction mixture. After 5 h the mixture was cooled to room temperature and treated with water. The aqueous phase was washed twice with ethyl acetate and then acidified with 1M hydrochloric acid to pH2-3. The aqueous phase was extracted with ethyl acetate, the combined organic phases were dried over MgSO₄ and the solvent was evaporated. The residue was purified by HPLC chromatography to yield 7.8 mg [2-(1H-indazol-3-yl)-7,7-dimethyl-6-oxo-6,7-dihydro-3H-imidazo[4,5-f]indol-5-yl]-acetic acid (0.021 mmol, 17%).

MS: M=373.9 (API−)

¹H-NMR (400 MHz, DMSO): δ (ppm)=1.37 (s, 6H), 4.49 (d, 2H), 6.97 and 7.29 (s, 1H), 7.29 (t, 1H), 7.44 and 7.73 (s, 1H), 7.46 (t, 1H), 7.64 (d, 1H), 8.49 (d, 1H), 12.92 and 12.99 (s, 1H), 13.53 and 13.58 (s, 1H)

### Example 37

### 2-[2-(1H-Indazol-3-yl)-7,7-dimethyl-6-oxo-6,7-dihydro-3H-imidazo[4,5-f]indol-5-yl]-acetamide

A mixture of [2-(1H-indazol-3-yl)-7,7-dimethyl-6-oxo-6,7-dihydro-3H-imidazo[4,5-f]indol-5-yl]-acetic acid ethyl ester (example 23, 100 mg, 0.248 mmol), methanol (1 drop) and ammonia (25%, 910 μl, 13.5 mmol) was stirred at room temperature. After 12 h further ammonia (25%, 910 μl, 13.5 mmol) was added. After 5 h the suspension was treated with water and the aqueous phase was extracted three times with ethyl acetate. The combined organic phases were dried over MgSO₄ and the solvent was evaporated. The residue was purified by HPLC chromatography to yield 54 mg 2-[2-(1H-indazol-3-yl)-7,7-dimethyl-6-oxo-6,7-dihydro-3H-imidazo[4,5-f]indol-5-yl]-acetamide (0.143 mmol, 58%).

MS: M=375.0 (API+)

¹H-NMR (400 MHz, DMSO): δ (ppm)=1.37 (s, 6H), 4.34 (s, 2H), 6.89 and 7.17 (s, 1H), 7.26 and 7.66 (s, 2H), 7.29 (t, 1H), 7.44 and 7.72 (s, 1H), 7.46 (t, 1H), 7.64 (d, 1H), 8.50 (d, 1H), 12.90 and 12.98 (s, 1H), 13.54 (s, 1H)

### Example 38

### N-(2-Dimethylamino-ethyl)-2-[2-(1H-indazol-3-yl)-7,7-dimethyl-6-oxo-6,7-dihydro-3H-imidazo[4,5-f]indol-5-yl]-acetamide

A mixture of [2-(1H-indazol-3-yl)-7,7-dimethyl-6-oxo-6,7-dihydro-3H-imidazo[4,5-f]indol-5-yl]-acetic acid ethyl ester (example 23, 50 mg, 0.124 mmol), N,N′-dimethylethylendiamine (159 μl, 1.37 mmol) and ammonium chloride (2 mg, 0.037 mmol) was heated to 105° C. in a sealed tube. After 1 h the reaction mixture was cooled to room temperature and treated with water. The precipitate formed was filtered off and washed with water. The combined aqueous phases were extracted three times with ethyl acetate. The combined organic phases were dried over MgSO₄ and the solvent was evaporated. The residue was purified by HPLC chromatography to yield 38.5 mg N-(2-dimethylamino-ethyl)-2-[2-(1H-indazol-3-yl)-7,7-dimethyl-6-oxo-6,7-dihydro-3H-imidazo[4,5-f]indol-5-yl]-acetamide (0.086 mmol, 70%).

MS: M=446.2 (API+)

¹H-NMR (400 MHz, DMSO): δ (ppm)=1.37 (s, 6H), 2.15 (bs, 3H), 2.18 (bs, 3H), 2.32 (t, 2H), 3.20 (t, 2H), 4.37 (s, 2H), 6.88 and 7.17 (s, 1H), 7.29 (t, 1H), 7.44 and 7.72 (s, 1H), 7.46 (t, 1H), 7.64 (d, 1H), 8.13 (m, 1H), 8.49 (d, 1H), 12.90 and 12.98 (s, 1H), 13.53 and 13.57 (s, 1H)

In an analogous manner as described for example 38 the following examples 39-47 were prepared from the appropriate starting materials:

| Example | | ¹H-NMR(400MHz, | |
|---|---|---|---|
| No | Systematic Name | DMSO): δ(ppm)= | MS: M= |
| 39 | N-Benzyl-2-[2-(1H- | 1.38(s, 6H), 4.34(m, 2H), | 465.0(API+) |
| | indazol-3-yl)-7,7- | 4.46(s, 2H), 6.96 and | |
| | dimethyl-6-oxo-6,7- | 7.27(s, 1H), 7.19-7.38(m, 6H), | |
| | dihydro-3H- | 7.46(d, 1H), 7.49 and | |
| | imidazo[4,5-f]indol-5- | 7.73(s, 1H), 7.64(d, 1H), 8.50(t, | |
| | yl]-acetamide | 1H), 8.74(bd, 1H), 12.93 | |
| | | and 12.99(s, 1H), 13.54 and | |
| | | 13.58(s, 1H) | |
| 40 | 2-(1H-Indazol-3-yl)-7,7- | 1.37(s, 6H), 3.45(m, 2H), | 445.0(API+) |
| | dimethyl-5-(2- | 3.62(m, 4H), 3.70(m, 2H), | |
| | morpholin-4-yl-2-oxo- | 4.69(d, 2H), 6.96 an 7.29(s, | |
| | ethyl)-5,7-dihydro-3H- | 1H), 7.29(t, 1H), 7.43 and | |
| | imidazo[4,5-f]indol-6- | 7.72(s, 1H), 7.46(t, 1H), | |
| | one | 7.64(d, 1H), 8.49(s, 1H), | |
| | | 12.89 and 12.97(s, 1H), | |
| | | 13.52 and 13.57(s, 1H) | |
| 41 | 2-[2-(1H-Indazol-3-yl)- | 1.38(bs, 6H), 4.37(m, 2H), | 466.1(API+) |
| | 7,7-dimethyl-6-oxo-6,7- | 4.47(s, 2H), 6.95 and | |
| | dihydro-3H- | 7.24(s, 1H), 7.30(m, 1H), | |
| | imidazo[4,5-f]indol-5- | 7.37(m, 1H), 7.46(d, 1H), 7.48 | |
| | yl]-N-pyridin-3- | and 7.69(s, 1H), 7.64(d, | |
| | ylmethyl-acetamide | 1H), 7.70(m, 1H), | |
| | | 8.44-8.56(m, 3H), 8.80(t, 1H), | |
| | | 12.93 and 12.99(s, 1H), | |
| | | 13.54 and 13.58(s, 1H) | |
| 42 | 2-(1H-Indazol-3-yl)-7,7- | 1.37(s, 6H), 2.22(s, 3H), | 458.2(API+) |
| | dimethyl-5-[2-(4- | 2.29(bt, 2H), 2.41(bt, 2H), | |
| | methyl-piperazin-1-yl)- | 3.46(bt, 2H), 3.60(bt, 2H), | |
| | 2-oxo-ethyl]-5,7- | 4.68(bs, 2H), 6.93 and | |
| | dihydro-3H- | 7.25(s, 1H), 7.29(t, 1H), 7.43 | |
| | imidazo[4,5-f]indol-6- | and 7.72(s, 1H), 7.46(t, | |
| | one | 1H), 7.64(d, 1H), 8.49(d, | |
| | | 1H), 12.86 and 12.97(s, | |
| | | 1H), 13.55(bs, 1H) | |
| 43 | 2-[2-(1H-Indazol-3-yl)- | 1.40(s, 6H), 4.63(s, 2H), | 451.1(API+) |
| | 7,7-dimethyl-6-oxo-6,7- | 6.98-7.76(m, 2H), 7.07(t, | |
| | dihydro-3H- | 1H), 7.23-7.36(m, 3H), | |
| | imidazo[4,5-f]indol-5- | 7.46(t, 1H), 7.58-7.67(m, | |
| | yl]-N-phenyl-acetamide | 3H), 8.48(m, 1H), 10.41(d, | |
| | | 1H), 12.89 and 12.99(s, | |
| | | 1H), 13.52 and 13.57(s, 1H) | |
| 44 | 2-(1H-Indazol-3-yl)-7,7- | 1.37(s, 6H), 1.46(m, 2H), | 443.1(API+) |
| | dimethyl-5-(2-oxo-2- | 1.62(m, 4H), 3.44(m, 2H), | |
| | piperidin-1-yl-ethyl)- | 3.55(m, 2H), 4.65(s, 2H), | |
| | 5,7-dihydro-3H- | 6.93 and 7.24(s, 1H), | |
| | imidazo[4,5-f]indol-6- | 7.29(t, 1H), 7.43 and 7.72(s, | |
| | one | 1H), 7.46(t, 1H), 7.64(d, | |
| | | 1H), 8.49(d, 1H), 12.85 and | |
| | | 12.97(s, 1H), 13.51 and | |
| | | 13.57(s, 1H) | |
| 45 | N-(4-Fluoro-phenyl)-2- | 1.39(s, 6H), 4.62(s, 2H), | 469.2(ESI+) |
| | [2-(1H-indazol-3-yl)- | 7.00 and 7.29(s, 1H), | |
| | 7,7-dimethyl-6-oxo-6,7- | 7.16(m, 2H), 7.29(m, 1H), 7.46 | |
| | dihydro-3H- | and 7.74(s, 1H), 7.46(m, | |
| | imidazo[4,5-f]indol-5- | 1H), 7.36(m, 3H), 8.48(m, | |
| | yl]-acetamide | 1H), 10.48(m, 1H), 12.89 | |
| | | and 12.99(s, 1H), 13.52 and | |
| | | 13.57(s, 1H) | |
| 46 | N-(4-Fluoro-benzyl)-2- | 1.38(s, 6H), 4.32(m, 2H), | 483.0(ESI+) |
| | [2-(1H-indazol-3-yl)- | 4.45(s, 2H), 6.94 and | |
| | 7,7-dimethyl-6-oxo-6,7- | 7.21(s, 1H), 7.11-7.20(m, 2H), | |
| | dihydro-3H- | 7.26-7.40(m, 3H), | |
| | imidazo[4,5-f]indol-5- | 7.46(m, 1H), 7.48 and 7.73(s, | |
| | yl]-acetamide | 1H), 7.64(d, 1H), 8.50(m, | |
| | | 1H), 8.75(m, 1H), 12.93 | |
| | | and 12.99(s, 1H), 13.54 and | |
| | | 13.58(s, 1H) | |
| 47 | N-(3,5-Dimethoxy- | 1.37(s, 6H), 3.70(s, 3H), | 225.1(ESI+) |
| | benzyl)-2-[2-(1H- | 3.73(s, 3H), 4.27(m, 2H), | |
| | indazol-3-yl)-7,7- | 4.47(m, 2H), 6.36(s, 1H), | |
| | dimethyl-6-oxo-6,7- | 6.46(m, 2H), 6.96 and | |
| | dihydro-3H- | 7.23(s, 1H), 7.30(m, 1H), | |
| | imidazo[4,5-f]indol-5- | 7.45(m, 1H), 7.48 and 7.72(m, | |
| | yl]-acetamide | 1H), 7.64(m, 1H), 8.50(d, | |
| | | 1H), 8.69(m, 1H), 12.89 | |
| | | and 12.98(s, 1H), 13.53 and | |
| | | 13.57(s, 1H) | |

### Example 48

### N-(2,3-Dihydroxy-propyl)-2-[2-(1H-indazol-3-yl)-7,7-dimethyl-6-oxo-6,7-dihydro-3H-imidazo[4,5-f]indol-5-yl]-acetamide

In an analogous manner as described for example 38, N-(2,3-dihydroxy-propyl)-2-[2-(1H-indazol-3-yl)-7,7-dimethyl-6-oxo-6,7-dihydro-3H-imidazo[4,5-f]indol-5-yl]-acetamide was prepared using 2,2-dimethyl-1,3-dioxolane-4-methanamine instead of N,N′-dimethylethylendiamine.

MS: M=449.0 (API+)

¹H-NMR (400 MHz, DMSO): δ (ppm)=1.37 (s, 6H), 3.03 (m, 1H), 3.28-3.56 (m, 4H), 4.41 (s, 2H), 4.55 (bt, 1H), 4.82 (bd, 1H), 6.90 and 7.17 (s, 1H), 7.29 (t, 1H), 7.43 and 7.72 (s, 1H), 7.46 (t, 1H), 7.64 (d, 1H), 8.17 (bt, 1H), 8.49 (d, 1H), 12.89 and 12.98 (s, 1H), 13.55 (bs, 1H)

### Example 49

### N-Hydroxy-2-[2-(1H-indazol-3-yl)-7,7-dimethyl-6-oxo-6,7-dihydro-3H-imidazo[4,5-f]indol-5-yl]-acetamide

A mixture of [2-(1H-indazol-3-yl)-7,7-dimethyl-6-oxo-6,7-dihydro-3H-imidazo[4,5-f]indol-5-yl]-acetic acid ethyl ester (example 23, 100 mg, 0.248 mmol), hydroxylamine (2M in MeOH, 1240 μl, 2.48 mmol) and potassium hydroxide (15.5 mg, 0.276 mmol) was stirred under an argon atmosphere for 1.5 h at room temperature. The solvent was evaporated and the residue dissolved in water. The aqueous phase was extracted three times with ethyl acetate. The combined organic phases were dried over MgSO₄ and the solvent was evaporated. The residue was triturated with diisopropyl ether and dried in vacuum to yield 52 mg N-hydroxy-2-[2-(1H-indazol-3-yl)-7,7-dimethyl-6-oxo-6,7-dihydro-3H-imidazo[4,5-f]indol-5-yl]-acetamide (0.133 mmol, 54%).

MS: M=391.0 (API+)

¹H-NMR (400 MHz, DMSO): δ (ppm)=1.37 (s, 6H), 4.32 (s, 2H), 6.97 and 7.25 (s, 1H), 7.29 (t, 1H), 7.44 and 7.72 (s, 1H), 7.46 (t, 1H), 7.64 (d, 1H), 8.50 (d, 1H), 9.02 (s, 1H), 10.90 (d, 1H), 12.96 and 12.99 (s, 1H), 13.53 and 13.58 (s, 1H)

### Example 50

### N-Benzyloxy-2-[2-(1H-indazol-3-yl)-7,7-dimethyl-6-oxo-6,7-dihydro-3H-imidazo[4,5-f]indol-5-yl]-acetamide

To a solution of N-hydroxy-2-[2-(1H-indazol-3-yl)-7,7-dimethyl-6-oxo-6,7-dihydro-3H-imidazo[4,5-f]indol-5-yl]-acetamide (example 49, 20 mg, 0.051 mmol) in ethanol (0.5 ml) was added a solution of potassium hydroxide (3.5 mg, 0.054 mmol) in water. After 5 minutes benzyl bromide (10.1 mg, 0.059 mmol) was added and the reaction mixture was stirred at room temperature under an argon atmosphere. After 5 h further 0.2 equivalents benzyl bromide were added and stirring was continued overnight. The solvent was evaporated and the residue was triturated with diethyl ether. The precipitate was filtered off and purified by HPLC chromatography to yield 9 mg N-benzyloxy-2-[2-(1H-indazol-3-yl)-7,7-dimethyl-6-oxo-6,7-dihydro-3H-imidazo[4,5-f]indol-5-yl]-acetamide (0.019 mmol, 37%)

MS: M=481.1 (API+)

¹H-NMR (400 MHz, DMSO): δ (ppm)=1.38 (s, 6H), 4.33 (s, 2H), 4.84 (s, 2H), 6.98 and 7.23 (s, 1H), 7.30 (t, 1H), 7.34-7.50 (m, 6H), 7.53 and 7.73 (s, 1H), 7.64 (d, 1H), 8.51 (m, 1H), 11.54 (bs, 1H), 12.95 (bs, 1H), 13.56 (bs, 1H)

### Example 51

### 2-[2-(1H-Indazol-3-yl)-7,7-dimethyl-6-oxo-6,7-dihydro-3H-imidazo[4,5-f]indol-5-yl]-N-methoxy-acetamide

To a solution of O-methylhydroxylamine hydrochloride (18 mg, 0.215 mmol) in dichloromethane (2 ml) was added triethylamine (21.8 mg, 30 μl, 0.215 mmol) and then [2-(1H-indazol-3-yl)-7,7-dimethyl-6-oxo-6,7-dihydro-3H-imidazo[4,5-f]indol-5-yl]-acetic acid (example 36, 80 mg, 0.213 mmol), N′-(3-dimethylaminopropyl)-N-ethylcarbodiimide hydrochloride (49 mg, 0.256 mmol) and hydroxybenzotriazole hydrate (39 mg, 0.255 mmol). After 3.5 h at room temperature the solvent was evaporated, the residue treated with saturated bicarbonate solution and the aqueous phase extracted three times with ethyl acetate. The combined organic phases were dried over MgSO₄ and the solvent was evaporated. The residue was purified by HPLC chromatography to yield 2.4 mg 2-[2-(1H-Indazol-3-yl)-7,7-dimethyl-6-oxo-6,7-dihydro-3H-imidazo[4,5-f]indol-5-yl]-N-methoxy-acetamide (0.006 mmol, 2.8%)

MS: M=405.0 (API+)

¹H-NMR (400 MHz, DMSO): δ (ppm)=1.37 (s, 6H), 3.65 (s, 3H), 4.31 (s, 2H), 6.96 and 7.24 (s, 1H), 7.29 (t, 1H), 7.44 and 7.73 (s, 1H), 7.46 (t, 1H), 7.64 (d, 1H), 8.49 (d, 1H), 11.51 (bs, 1H), 12.94 and 12.99 (s, 1H), 13.54 and 13.58 (s, 1H)

### Example 52

### 5-(2-Amino-ethyl)-2-(1H-indazol-3-yl)-7,7-dimethyl-5,7-dihydro-3H-imidazo[4,5-f]indol-6-one

[2-(1H-Indazol-3-yl)-7,7-dimethyl-6-oxo-6,7-dihydro-3H-imidazo[4,5-f]indol-5-yl]-acetonitrile (1645 mg, 4.61 mmol) was hydrogenated in 2M methanolic ammonia in the presence of Raney-Nickel (1650 mmg, 280 mmol) for 13 h at 30 mbar. The catalyst was filtered off and the solvent evaporated. The residue was triturated with water and in dried in vacuum to yield 1200 mg 5-(2-amino-ethyl)-2-(1H-indazol-3-yl)-7,7-dimethyl-5,7-dihydro-3H-imidazo[4,5-f]indol-6-one (3.33 mmol, 72%)

MS: M=361.2 (API+)

¹H-NMR (400 MHz, DMSO): δ (ppm)=1.34 (s, 6H), 2.82 (t, 2H), 3.75 (t, 2H), 7.05-7.76 (m, 2H), 7.30 (t, 1H), 7.46 (t, 1H), 7.64 (d, 1H), 8.50 (d, 1H)

### Example 53

### N-{2-[2-(1H-Indazol-3-yl)-7,7-dimethyl-6-oxo-6,7-dihydro-3H-imidazo[4,5-f]indol-5-yl]-ethyl}-benzamide

To a solution of benzoic acid (6.5 mg, 0.053 mmol) in dichloromethane (1 ml) were added N′-(3-dimethylaminopropyl)-N-ethylcarbodiimide hydrochloride (15 mg, 0.078 mmol) and hydroxybenzotriazole hydrate (12 mg, 0.078 mmol). After 50 minutes at room temperature a solution of 5-(2-amino-ethyl)-2-(1H-indazol-3-yl)-7,7-dimethyl-5,7-dihydro-3H-imidazo[4,5-f]indol-6-one (example 52, 18.7 mg, 0.052 mmol) in DMF (1 ml) was added and stirring continued for 2 h. The solvent was evaporated, the residue treated with saturated bicarbonate solution and the aqueous phase extracted three times with ethyl acetate. The combined organic phases were dried over MgSO₄ and the solvent was evaporated. The residue was purified by HPLC chromatography to yield 6.8 mg N-{2-[2-(1H-Indazol-3-yl)-7,7-dimethyl-6-oxo-6,7-dihydro-3H-imidazo[4,5-f]indol-5-yl]-ethyl}-benzamide (0.015 mmol, 28%).

MS: M=465.1 (API+)

¹H-NMR (400 MHz, DMSO): δ (ppm)=1.32 (s, 6H), 3.58 (bt, 2H), 3.94 (bt, 2H), 7.13-7.81 (m, 2H), 7.30 (bt, 1H), 7.37-7.55 (m, 5H), 7.64 (d, 1H), 7.76 (d, 1H), 8.50 (m, 1H), 8.64 (bt, 1H), 12.97 (s, 1H), 13.54 (s, 1H)

In an analogous manner as described for example 53 the following example 54 was prepared from the appropriate starting materials:

### Example 54

### N-{2-[2-(1H-Indazol-3-yl)-7,7-dimethyl-6-oxo-6,7-dihydro-3H-imidazo[4,5-f]indol-5yl]-ethyl}-2-phenyl-acetamide

MS: M=479.1 (API+)

¹H-NMR (400 MHz, DMSO): δ (ppm)=1.35 (s, 6H), 3.33 (s, 2H), 3.38 (m, 2H), 3.84 (t, 1H), 7.16 (m, 5H), 7.31 (s, 1H), 7.40 (t, 1H), 7.54 (t, 1H), 7.67 (s, 1H), 7.74 (d, 1H), 8.25 (t, 1H), 8.51 (d, 1H), 14.09 (s, 1H)

### Example 55

### N-{2-[2-(1H-Indazol-3-yl)-7,7-dimethyl-6-oxo-6,7-dihydro-3H-imidazo[4,5-f]indol-5-yl]-ethyl}-nicotinamide

To a solution of 5-(2-amino-ethyl)-2-(1H-indazol-3-yl)-7,7-dimethyl-5,7-dihydro-3H-imidazo[4,5-f]indol-6-one (example 52, 50 mg, 0.139 mmol) in THF (2 ml) and DMF (0.3 ml) at 0° C. were added nicotinyl chloride hydrochloride (50 mg, 0.281 mmol) and diisopropylethylamine (82 mg, 0.632 mmol). After 5 h at room temperature the solvent was evaporated and methanol (1 ml) and KOH (1M solution, 1 ml) were added. After 30 minutes at room temperature the solvent was evaporated and the residue purified by HPLC chromatography to yield 36.5 mg N-{2-[2-(1H-indazol-3-yl)-7,7-dimethyl-6-oxo-6,7-dihydro-3H-imidazo[4,5-f]indol-5-yl]-ethyl}-nicotinamide (0.078 mmol, 56%).

MS: M=466.2 (ESI+)

¹H-NMR (400 MHz, DMSO): δ (ppm)=1.31 (bs, 6H), 3.60 (m, 2H), 3.96 (t, 2H), 7.14 and 7.42 (s, 1H), 7.30 (m, 1H), 7.45 and 7.70 (s, 1H), 7.47 (m, 2H), 7.64 (m, 1H), 8.07 (d, 1H), 8.50 (t, 1H), 8.65 (m, 1H), 8.80-8.92 (m, 2H), 12.97 (s, 1H), 13.53 and 13.58 (s, 1H)

In an analogous manner as described for example 55 the following examples 56-61 were prepared from the appropriate acyl chlorides, carbamoyl chlorides and sulfonyl chlorides:

| | | ¹H-NMR(400MHz, | |
|---|---|---|---|
| Example No | Systematic Name | DMSO): δ(ppm)= | MS: M= |
| 56 | Cyclopropanecarboxylic | 0.57(m, 2H), 0.64(m, 2H), | 429.2(ESI+) |
| | acid {2-[2-(1H-indazol- | 1.34(m, 6H), 1.43(m, 1H), | |
| | 3-yl)-7,7-dimethyl-6- | 3.35(m, 2H), 3.79(t, 2H), | |
| | oxo-6,7-dihydro-3H- | 7.08 and 7.41(s, 1H), | |
| | imidazo[4,5-f]indol-5- | 7.30(m, 1H), 7.41 and 7.70(s, | |
| | yl]-ethyl}-amide | 1H), 7.46(t, 1H), 7.64(d, | |
| | | 1H), 8.23(m, 1H), 8.51(m, | |
| | | 1H), 12.95(s, 1H), 13.52 | |
| | | and 13.57(s, 1H) | |
| 57 | Morpholine-4- | 1.34(s, 6H), 3.14-3.22(m, | 472.1(API−) |
| | carboxylic acid {2-[2- | 6H), 3.44(m, 4H), 3.80(bt, | |
| | (1H-indazol-3-yl)-7,7- | 2H), 6.74(m, 1H), 7.11 and | |
| | dimethyl-6-oxo-6,7- | 7.41(s, 1H), 7.29(t, 1H), | |
| | dihydro-3H- | 7.43 and 7.69(s, 1H), | |
| | imidazo[4,5-f]indol-5- | 7.46(t, 1H), 7.64(d, 1H), | |
| | yl]-ethyl}-amide | 8.51(m, 1H), 12.96(s, 1H), | |
| | | 13.52 and 13.57(s, 1H) | |
| 58 | Pyrrolidine-1-carboxylic | 1.34(s, 6H), 1.69(m, 4H), | 456.28(ESI−) |
| | acid {2-[2-(1H-indazol- | 3.13(m, 4H), 3.29(m, 2H), | |
| | 3-yl)-7,7-dimethyl-6- | 3.79(t, 2H), 6.29(m, 1H), | |
| | oxo-6,7-dihydro-3H- | 7.14 and 7.41(s, 1H), | |
| | imidazo[4,5-f]indol-5- | 7.29(m, 1H), 7.46 and 7.68(s, | |
| | yl]-ethyl}-amide | 1H), 7.46(m, 1H), 7.64(m, | |
| | | 1H), 8.51(m, 1H), 12.95 | |
| | | and 12.97(s, 1H), 13.51 and | |
| | | 13.56(s, 1H) | |
| 59 | 4-Methyl-piperazine-1- | 1.34(m, 6H), 2.06(m, 3H), | 485.20(ESI−) |
| | carboxylic acid {2-[2- | 2.14(m, 4H), 3.21(m, 4H), | |
| | (1H-indazol-3-yl)-7,7- | 3.79(m, 2H), 6.70(t, 1H), | |
| | dimethyl-6-oxo-6,7- | 7.11 and 7.41(s, 1H), | |
| | dihydro-3H- | 7.29(m, 1H), 7.43 and 7.69(s, | |
| | imidazo[4,5-f]indol-5- | 1H), 7.46(m, 1H), 7.64(m, | |
| | yl]-ethyl}-amide | 1H), 8.51(m, 1H), 12.95(s, | |
| | | 1H), 13.51 and 13.56(s, 1H) | |
| 60 | N-{2-[2-(1H-Indazol-3- | 1.33(s, 6H), 3.02(m, 2H), | 523.1(API+) |
| | yl)-7,7-dimethyl-6-oxo- | 3.80(m, 2H), 7.07 and | |
| | 6,7-dihydro-3H- | 7.34(s, 1H), 7.30(t, 1H), 7.42 | |
| | imidazo[4,5-f]indol-5- | and 7.71(s, 1H), 7.47(t, | |
| | yl]-ethyl}- | 1H), 7.54-7.67(m, 5H), | |
| | benzenesulfonamide | 7.81(m, 2H), 8.51(m, 1H), | |
| | | 13.00(s, 1H), 13.56(s, 1H) | |
| 61 | N-{2-[2-(1H-Indazol-3- | 1.35(s, 6H), 2.91(s, 3H), | 439.3(ESI+) |
| | yl)-7,7-dimethyl-6-oxo- | 3.24(m, 2H), 3.85(m, 2H), | |
| | 6,7-dihydro-3H- | 7.10 and 7.40(s, 1H), | |
| | imidazo[4,5-f]indol-5- | 7.30(m, 2H), 7.44 and 7.72(s, | |
| | yl]-ethyl}- | 1H), 7.46(m, 1H), 7.64(d, | |
| | methanesulfonamide | 1H), 8.50(m, 1H), 12.99(s, | |
| | | 1H), 13.54 and 13.58(s, 1H) | |

### Example 62

### N-{2-[2-(1H-Indazol-3-yl)-7,7-dimethyl-6-oxo-6,7-dihydro-3H-imidazo[4,5-f]indol-5-yl]-ethyl}-acetamide

To a solution of 5-(2-amino-ethyl)-2-(1H-indazol-3-yl)-7,7-dimethyl-5,7-dihydro-3H-imidazo[4,5-f]indol-6-one (example 52, 50 mg, 0.139 mmol) in pyridine (0.5 ml) was added acetic anhydride (142 mg, 131 μl, 1.39 mmol). After 2 h at room temperature the reaction mixture was treated with water and the solvent was evaporated. To the residue methanol (1 ml) and KOH (1M solution, 1 ml) were added. After 90 minutes at room temperature the solvent was evaporated and the residue purified by HPLC chromatography to yield 16.5 mg N-{2-[2-(1H-Indazol-3-yl)-7,7-dimethyl-6-oxo-6,7-dihydro-3H-imidazo[4,5-f]indol-5-yl]-ethyl}-acetamide (0.041 mmol, 30%).

MS: M=403.3 (ESI+)

¹H-NMR (400 MHz, DMSO): δ (ppm)=1.33 (bs, 3H), 1.35 (bs, 3H), 1.73 and 1.75 (s, 3H), 3.30 (m, 2H), 3.79 (m, 2H), 7.07 and 7.40 (s, 1H), 7.30 (m, 1H), 7.42 and 7.70 (s, 1H), 7.46 (m, 1H), 7.64 (m, 1H), 8.02 (m, 1H), 8.50 (m, 1H), 12.96 (s, 1H), 13.52 and 13.57 (s, 1H)

### Example 63

### 1-Benzyl-3-{2-[2-(1H-indazol-3-yl)-7,7-dimethyl-6-oxo-6,7-dihydro-3H-imidazo[4,5-f]indol-5-yl]-ethyl}-urea

To a solution of 5-(2-amino-ethyl)-2-(1H-indazol-3-yl)-7,7-dimethyl-5,7-dihydro-3H-imidazo[4,5-f]indol-6-one (example 52, 100 mg, 0.277 mmol) in DMF (2 ml) were added triethylamine (55.9 mg, 77 μl, 0.552 mmol) and benzyl isocyanate (41 mg, 0.308 mmol) and heated under reflux for 6h under an argon atmosphere. After cooling to room temperature the reaction mixture was treated with water and the aqueous phase was extracted three times with ethyl acetate. The combined organic phases were washed with brine, dried over MgSO₄ and the solvent was evaporated. The residue was purified by HPLC chromatography to yield 37 mg 1-benzyl-3-{2-[2-(1H-indazol-3-yl)-7,7-dimethyl-6-oxo-6,7-dihydro-3H-imidazo[4,5-f]indol-5-yl]-ethyl}-urea (0.075 mmol, 27%).

MS: M=492.51 (ESI−)

¹H-NMR (400 MHz, DMSO): δ (ppm) 1.34 (m, 6H), 3.33 (m, 2H), 3.79 (t, 2H), 4.19 (m, 2H), 6.11 (t, 1H), 6.41 (m, 1H), 7.11-7.72 (m, 2H), 7.11-7.34 (m, 6H), 7.47 (m, 1H), 7.64 (m, 1H), 8.51 (m, 1H), 12.95 and 12.97 (s, 1H), 13.51 and 13.57 (s, 1H)

### Example 64

### 2-(1H-Indazol-3-yl)-5-(2-methanesulfinyl-ethyl)-7,7-dimethyl-5,7-dihydro-3H-imidazo[4,5-f]indol-6-one

To a solution of 2-(1H-indazol-3-yl)-7,7-dimethyl-5-(2-methylsulfanyl-ethyl)-5,7-dihydro-3H-imidazo[4,5-f]indol-6-one (example 25, 40 mg, 0.102 mmol) in dichloromethane (1.5 ml) was added a solution of 3-chloroperoxybenzoic acid (18.3 mg, 0.082 mmol) in dichloromethane (0.5 ml). After 15 minutes the solvent was evaporated and the residue purified by HPLC chromatography to yield 21 mg 2-(1H-Indazol-3-yl)-5-(2-methanesulfinyl-ethyl)-7,7-dimethyl-5,7-dihydro-3H-imidazo[4,5-f]indol-6-one (0.052 mmol, 50%).

MS: M=408.0 (ESI+)

¹H-NMR (400 MHz, DMSO): δ (ppm)=1.36 (s, 6H), 2.63 (s, 3H), 3.04 (m, 2H), 4.15 (t, 2H), 7.16 and 7.74 (s, 1H, two tautomeric forms), 7.30 (t, 1H), 7.47 (m, 2h), 7.65 (d, 1H), 8.51 (d, 1H), 13.01 (s, 1H), 13.59 (s, 1H)

### Example 65

### 2-(1H-Indazol-3-yl)-5-(2-methanesulfonyl-ethyl)-7,7-dimethyl-5,7-dihydro-3H-imidazo[4,5-f]indol-6-one

To a solution of 2-(1H-indazol-3-yl)-7,7-dimethyl-5-(2-methylsulfanyl-ethyl)-5,7-dihydro-3H-imidazo[4,5-f]indol-6-one (example 25, 40 mg, 0.102 mmol) in dichloromethane (1.5 ml) was added a solution of 3-chloroperoxybenzoic acid (68.7 mg, 0.308 mmol) in dichloromethane (0.5 ml). After 2 h at room temperature the solvent was evaporated and the residue purified by HPLC chromatography to yield 25.3 mg 2-(1H-indazol-3-yl)-5-(2-methanesulfonyl-ethyl)-7,7-dimethyl-5,7-dihydro-3H-imidazo[4,5-f]indol-6-one (0.060 mmol, 58%).

MS: M=424.2 (ESI+)

¹H-NMR (400 MHz, DMSO): δ (ppm)=1.36 (s, 6H), 3.11 (m, 3H), 3.56 (m, 2H), 4.21 (m, 2H), 7.13 and 7.75 (s, 1H, two tautomeric forms), 7.31 (t, 1H), 7.47 (m, 2h), 7.65 (d, 1H), 8.51 (t, 1H), 12.99 and 13.02 (s, 1H, two tautomeric forms), 13.54 and 13.59 (s, 1H, two tautomeric forms)

### Example 66

### 5-Ethyl-2-(5-fluoro-1H-indazol-3-yl)-7,7-dimethyl-5,7-dihydro-3H-imidazo[4,5-f]indol-6-one

In an analogous manner as described for example 3, 5-ethyl-2-(5-fluoro-1H-indazol-3-yl)-7,7-dimethyl-5,7-dihydro-3H-imidazo[4,5-f]indol-6-one was prepared from 5,6-diamino-1-ethyl-3,3-dimethyl-1,3-dihydro-indol-2-one (see part A, starting materials) and 5-fluoro-1H-indazole-3-carboxylic acid (prepared from 5-fluoroisatin according to WO03/035065, reference example 26 and J. Am. Chem. Soc. 1952 (74), 2009-2012).

MS: M=364.3 (ESI+)

¹H-NMR (400 MHz, DMSO): δ (ppm)=1.21 (t, 3H), 1.33(s, 6H), 3.78 (bq, 2H), 7.03 and 7.39 (s, 1H), 7.39 (m, 1H), 7.44 and 7.74 (s, 1H), 7.70 (m, 1H), 8.13 (m, 1H), 12.97 and 13.03 (s, 1H), 13.69 (s, 1H)

In an analogous manner as described for example 66 the following examples 67-68 were prepared from the appropriate isatins:

| | | ¹H-NMR(400MHz, | |
|---|---|---|---|
| Example No | Systematic Name | DMSO): δ(ppm)= | MS: M= |
| 67 | 5-Ethyl-7,7-dimethyl-2- | 1.21(t, 3H), 1.33(s, 6H), | 430.0(API+) |
| | (5-trifluoromethoxy- | 3.79(m, 2H), 7.40 and | |
| | 1H-indazol-3-yl)-5,7- | 7.80(s, 1H, two tautomeric | |
| | dihydro-3H- | forms), 7.43-7.49(m, 2H), | |
| | imidazo[4,5-f]indol-6- | 7.78(m, 1H), 8.41(d, 1H), | |
| | one | 13.04 and 13.10(s, 1H, two | |
| | | tautomeric forms), 13.83 | |
| | | and 13.88(s, 1H, two | |
| | | tautomeric forms) | |
| 68 | 2-(5-Chloro-1H- | 1.21(m, 3H), 1.33(s, 6H), | 380.1(ESI+) |
| | indazol-3-yl)-5-ethyl- | 3.78(m, 2H), 7.03 and | |
| | 7,7-dimethyl-5,7- | 7.43(s, 1H), 7.45 and 7.77(s, | |
| | dihydro-3H- | 1H), 7.49(m, 1H), 7.70(m, | |
| | imidazo[4,5-f]indol-6- | 1H), 8.52(m, 1H), 13.01 | |
| | one | and 13.07(s, 1H), 13.76(bs, | |
| | | 1H) | |

### Example 69

### 3-(5-Ethyl-7,7-dimethyl-6-oxo-3,5,6,7-tetrahydro-imidazo[4,5-f]indol-2-yl)-1H-indazole-5-carboxylic acid

### i) 3-Formyl-1H-indazole-5-carboxylic acid

To a mixture of indole-5-carboxylic acid (5.5 g, 0.0338 mol) in water (250 ml) was added NaNO₂ (23.5 g, 0.338 mol) and hydrochloride solution (6N, 42 ml, 0.293 mol). After 12 h at room temperature the precipitate was filtered off, washed with water (270 ml) and dried at 50° C. to yield 5.36 g 3-formyl-1H-indazole-5-carboxylic acid (0.028 mol, 83%) which was used without further purification.

### ii) 3-(5-Ethyl-7,7-dimethyl-6-oxo-3,5,6,7-tetrahydro-imidazo[4,5-f]indol-2-yl)-1H-indazole-5-carboxylic acid

A mixture of 6,7-diamino-1-ethyl-4,4-dimethyl-3,4-dihydro-1H-quinolin-2-one (1.1 g, 0.005 mol), 3-formyl-1H-indazole-5-carboxylic acid (1.0 g, 0.005 mol) and sulfur (0.176 g, 0.005 mol) in DMF (25 ml) was heated under reflux for 4.5 h. After cooling to room temperature, the reaction mixture was poured into water. After stirring for 15 minutes the precipitate was filtered off, washed thoroughly with water and dried in vacuo over P₂O₅ to yield 1.74 g 3-(5-Ethyl-7,7-dimethyl-6-oxo-3,5,6,7-tetrahydro-imidazo[4,5-f]indol-2-yl)-1H-indazole-5-carboxylic acid (0.004 mol, 87%).

MS: M=390.4 (ESI+)

¹H-NMR (400 MHz, DMSO): δ (ppm)=1.21 (t, 3H), 1.34 (s, 6H), 3.79 (b, 2H), 7.04 and 7.46 (s, 1H, two tautomeric forms), 7.51 and 7.84 (s, 1H, two tautomeric forms), 7.70 (d, 1H), 8.02 (d, 1H), 9.22 and 9.24 (s, 1H, two tautomeric forms), 12.87 (br, 1H), 13.05 and 13.11 (s, 1H, two tautomeric forms), 13.82 and 13.86 (s, 1H, two tautomeric forms)

In an analogous manner as described for example 69 the following examples 70-74 were prepared from the appropriate indoles:

| | | ¹H-NMR(400MHz, | |
|---|---|---|---|
| Example No | Systematic Name | DMSO): δ(ppm)= | MS: M= |
| 70 | 2-(6-Bromo-1H- | 1.20(t, 3H), 1.33(s, 6H), | 425.6(API+) |
| | indazol-3-yl)-5-ethyl- | 3.78(m, 2H), 7.03 and | |
| | 7,7-dimethyl-5,7- | 7.37(s, 1H), 7.44 and 7.72(s, | |
| | dihydro-3H- | 1H), 7.45(m, 1H), 7.89(m, | |
| | imidazo[4,5-f]indol-6- | 1H), 8.44(m, 1H), 13.01 | |
| | one | and 13.07(s, 1H), 13.67 and | |
| | | 13.71(s, 1H) | |
| 71 | 5-Ethyl-7,7-dimethyl-2- | 1.21(m, 3H), 1.34(m, 6H), | 391.04(ES+) |
| | (5-nitro-1H-indazol-3- | 3.79(m, 2H), 7.05 and | |
| | yl)-5,7-dihydro-3H- | 7.48(s, 1H), 7.52 and 7.87(s, | |
| | imidazo[4,5-f]indol-6- | 1H), 7.85(m, 1H), 8.31(m, | |
| | one | 1H), 9.44(m, 1H), 13.19 | |
| | | and 13.25(s, 1H), 14.19(s, | |
| | | 1H) | |
| 72 | 3-(5-Ethyl-7,7-dimethyl- | 1.21(m, 3H), 1.34(s, 6H), | 371.06(ES+) |
| | 6-oxo-3,5,6,7- | 3.79(m, 2H), 7.05 and | |
| | tetrahydro-imidazo[4,5- | 7.44(s, 1H), 7.47 and 7.79(s, | |
| | f]indol-2-yl)-1H- | 1H), 7.83(m, 2H), 8.95(m, | |
| | indazole-5-carbonitrile | 1H), 13.14 and 13.20(s, | |
| | | 1H), 14.06 and 14.09(s, 1H) | |
| 73 | 2-(5-Bromo-1H- | 1.21(m, 3H), 1.33(s, 6H), | 423.9(ESI−) |
| | indazol-3-yl)-5-ethyl- | 3.78(m, 2H), 7.03 and | |
| | 7,7-dimethyl-5,7- | 7.44(s, 1H), 7.45 and 7.78(s, | |
| | dihydro-3H- | 1H), 7.58(m, 1H), 7.65(m, | |
| | imidazo[4,5-f]indol-6- | 1H), 8.69(m, 1H), 13.00 | |
| | one | and 13.06(s, 1H), 13.73 and | |
| | | 13.77(s, 1H) | |
| 74 | 3-(5-Ethyl-7,7-dimethyl- | 1.21(t, 3H), 1.34(s, 6H), | 390.3(ESI+) |
| | 6-oxo-3,5,6,7- | 3.78(m, 2H), 7.04 and | |
| | tetrahydro-imidazo[4,5- | 7.40(s, 1H, two tautomeric | |
| | f]indol-2-yl)-1H- | forms), 7.46 and 7.74(s, 1H, | |
| | indazole-6-carboxylic | two tautomeric forms), | |
| | acid | 7.87(d, 1H), 8.23(s, 1H), | |
| | | 8.57(d, 1H), 13.02 and 13.08(br, | |
| | | 1H, two tautomeric forms), | |
| | | 13.12(br, 1H), 13.86 and | |
| | | 13.90(br, 1H, two | |
| | | tautomeric forms) | |

### Example 75

### 3-(5-Isopropyl-7,7-dimethyl-6-oxo-3,5,6,7-tetrahydro-imidazo[4,5-f]indol-2-yl)-1H-indazol-5-carboxylic acid

In an analogous manner as described for example 69ii, 3-(5-isopropyl-7,7-dimethyl-6-oxo-3,5,6,7-tetrahydro-imidazo[4,5-f]indol-2-yl)-1H-indazole-5-carboxylic acid was prepared from 3-formyl-1H-indazole-5-carboxylic acid (see example 69i) and 5,6-diamino-3,3-dimethyl-1-isopropyl-1,3-dihydro-indol-2-one (see part A, starting materials).

MS: M=404.2 (ESI+)

¹H-NMR (400 MHz, DMSO): δ (ppm)=1.32 (s, 6H), 1.48 (m, 6H), 4.53-4.70 (m, 1H), 7.15 and 7.45 (s, 1H, two tautomeric forms), 7.58 and 7.83 (s, 1H, two tautomeric forms), 7.71 (d, 1H), 8.02 (d, 1H), 9.23 (s, 1H), 12.90 (br, 1H), 12.97 and 13.09 (s, 1H, two tautomeric forms), 13.82 and 13.87 (s, 1H, two tautomeric forms)

### Example 76

### 3-(7,7-Dimethyl-6-oxo-3,5,6,7-tetrahydro-imidazo[4,5-f]indol-2-yl)-1H-indazole-5-carboxylic acid ethylamide

### i) 3-(7,7-Dimethyl-6-oxo-3,5,6,7-tetrahydro-imidazo[4,5-f]indol-2-yl)-1H-indazole-5-carboxylic acid

In an analogous manner as described for example 69ii, 3-(7,7-dimethyl-6-oxo-3,5,6,7-tetrahydro-imidazo[4,5-f]indol-2-yl)-1H-indazole-5-carboxylic acid was prepared from 3-formyl-1H-indazole-5-carboxylic acid (see example 69i) and 5,6-diamino-3,3-dimethyl-1,3-dihydro-indol-2-one (U.S. Pat. No. 4,666,923A) and was used without further purification.

### ii) 3-(7,7-Dimethyl-6-oxo-3,5,6,7-tetrahydro-imidazo[4,5-f]indol-2-yl)-1H-indazole-5-carboxylic acid ethylamide

A mixture of 3-(7,7-dimethyl-6-oxo-3,5,6,7-tetrahydro-imidazo[4,5-f]indol-2-yl)-1H-indazole-5-carboxylic acid (130 mg, 0.342 mmol), ethylamine (171 μl, 0.342 mmol), O-(7-azabenzotriazol-1-yl)-N,N,N′,N′-tetramethyluronium hexafluorophosphate (134 mg, 0.342 mmol), triethylamine (38 mg, 52.3 μl, 0.376 mmol) and DMF (2 ml) in a sealed tube was heated in a microwave at 100° C. for 15 minutes. The mixture was treated with water and the aqueous phase was extracted three times with ethyl acetate. The combined organic phases were washed with water, dried over MgSO₄ and the solvent was evaporated. The residue was purified by HPLC chromatography to yield 27 mg 3-(7,7-dimethyl-6-oxo-3,5,6,7-tetrahydro-imidazo[4,5-f]indol-2-yl)-1H-indazole-5-carboxylic acid ethylamide (0.069 mmol, 20%).

MS: M=389.1 (ESI+)

¹H-NMR (400 MHz, DMSO): δ (ppm)=1.18 (t, 3H), 1.33 (s, 6H), 3.34 (m, 2H), 6.95 and 7.16 (s, 1H, two tautomeric forms), 7.39 and 7.71 (s, 1H, two tautomeric forms), 7.65 (d, 1H), 7.91 (d, 1H), 8.59 (b, 1H), 8.98 (s, 1H), 10.30 (b, 1H), 12.83 (b, 1H), 13.71 (b, 1H)

In an analogous manner as described for example 76 the following examples 77-78 were prepared from the appropriate amines:

| | | ¹H-NMR(400MHz, | |
|---|---|---|---|
| Example No | Systematic Name | DMSO): δ(ppm)= | MS: M= |
| 77 | 3-(7,7-Dimethyl-6-oxo- | 1.32(s, 6H), 4.54(d, 2H), | 451.1(API+) |
| | 3,5,6,7-tetrahydro- | 6.94 and 7.15(s, 1H, two | |
| | imidazo[4,5-f]indol-2- | tautomeric forms), 7.25(m, | |
| | yl)-1H-indazole-5- | 1H), 7.35(m, 5H), 7.38 and | |
| | carboxylic acid | 7.69(s, 1H, two tautomeric | |
| | benzylamide | forms), 7.66(s, 1H), | |
| | | 7.97(m, 1H), 9.04 8(s, 1H), | |
| | | 9.17(m, 1H), 10.27 and 10.32(s, | |
| | | 1H, two tautomeric forms,), | |
| | | 13.72(br, 1H) | |
| 78 | 3-(7,7-Dimethyl-6-oxo- | 1.32(s, 6H), 6.95 and | 437.5(ESI+) |
| | 3,5,6,7-tetrahydro- | 7.16(s, 1H, two tautomeric | |
| | imidazo[4,5-f]indol-2- | forms), 7.12(t, 1H), 7.37(d, | |
| | yl)-1H-indazole-5- | 2H), 7.4 and 7.73(s, 1H, | |
| | carboxylic acid | two tautomeric forms), | |
| | phenylamide | 7.74(t, 1H), 7.83(d, 2H), | |
| | | 8.01(m, 1H), 9.09(s, 1H), 10.30 | |
| | | and 10.33(s, 1H, two | |
| | | tautomeric forms), | |
| | | 10.45(s, 1H), 12.87 and 13.03(s, | |
| | | 1H, two tautomeric forms), | |
| | | 13.80(br, 1H) | |

### Example 79

### 3-(5-Ethyl-7,7-dimethyl-6-oxo-3,5,6,7-tetrahydro-imidazo[4,5-f]indol-2-yl)-1H-indazole-5-carboxylic acid benzylamide

A mixture of 3-(5-ethyl-7,7-dimethyl-6-oxo-3,5,6,7-tetrahydro-imidazo[4,5-f]indol-2-yl)-1H-indazole-5-carboxylic acid (example 69, 120 mg, 0.308 mmol), 1,1′-carbonyl-diimidazole (60 mg, 0.370 mmol) and THF (10 ml) was heated under reflux for 1.5 h and then cooled to room temperature. Benzylamine (49.5 mg, 50.5 μl, 0.462 mmol) was added and the mixture was stirred overnight. The mixture was poured into water and the aqueous phase was extracted three times with ethyl acetate. The combined organic phases were washed with bicarbonate solution, water, diluted acetic acid, water, diluted ammonia and water and dried over MgSO₄. The solvent was evaporated. The residue was purified by silicagel chromatography (dichloromethane/methanol 98:2→90:10) to yield 52 mg 3-(5-ethyl-7,7-dimethyl-6-oxo-3,5,6,7-tetrahydro-imidazo[4,5-f]indol-2-yl)-1H-indazole-5-carboxylic acid benzylamide (0.105 mmol, 34%)

MS: M=479.2 (ESI+)

¹H-NMR (400 MHz, DMSO): δ (ppm)=1.21 (t, 3H), 1.33 (s, 6H), 3.78 (q, 2H), 4.54 (d, 2H), 7.04 and 7.44 (s, 1H, two tautomeric forms), 7.25 (t, 1H), 7.33-7.38 (m, 6H), 7.45 and 7.78 (s, 1H, two tautomeric forms), 7.68 (d, 1H), 7.98 (d, 1H), 9.05 and 9.07 (s, 1H, two tautomeric forms), 9.18 (t, 1H), 13.00 and 13.06 (s, 1H, two tautomeric forms), 13.76 (br, 1H)

In an analogous manner as described for example 79 the following examples 80-82 were prepared from the appropriate amines:

| | | ¹H-NMR(400MHz, | |
|---|---|---|---|
| Example No | Systematic Name | DMSO): δ(ppm)= | MS: M= |
| 80 | 3-(5-Ethyl-7,7-dimethyl- | 1.21(t, 3H), 1.33(s, 6H), | 480.2(ESI+) |
| | 6-oxo-3,5,6,7- | 3.78(q, 2H), 4.63(d, 2H), | |
| | tetrahydro-imidazo[4,5- | 7.04 and 7.37(s, 1H, two | |
| | f]indol-2-yl)-1H- | tautomeric forms), 7.28(t, | |
| | indazole-5-carboxylic | 1H), 7.39 and 7.78(s, 1H, | |
| | acid (pyridin-2- | two tautomeric forms), | |
| | ylmethyl)-amide; | 7.45(d, 1H), 7.70(d, 1H), | |
| | compound with acetic | 7.77(t, 1H); 8.01(d, 1H), | |
| | acid | 8.54(d, 1H), 9.08 and 9.11(s, | |
| | | 1H, two tautomeric forms), | |
| | | 9.24(t, 1H), 13.01 and | |
| | | 13.07(s, 1H, two tautomeric | |
| | | forms), 13.76(br, 1H) | |
| 81 | 3-(5-Ethyl-7,7-dimethyl- | 1.21(t, 3H), 1.33(s, 1H), | 480.3(ESI+) |
| | 6-oxo-3,5,6,7- | 3.78(q, 2H), 4.56(d, 2H), | |
| | tetrahydro-imidazo[4,5- | 7.04 and 7.44(s, 1H, two | |
| | f]indol-2-yl)-1H- | tautomeric forms), | |
| | indazole-5-carboxylic | 7.36-7.39(m, 1H), 7.46 and | |
| | acid (pyridin-3- | 7.78(s, 1H, two tautomeric | |
| | ylmethyl)-amide; | forms), 7.69(d, 1H), | |
| | compound with acetic | 7.77(d, 1H), 7.96(d, 1H), | |
| | acid | 8.47(d, 1H), 8.61(s, 1H), 9.24(t, | |
| | | 1H), 13.01 and 13.07(s, 1H, | |
| | | two tautomeric forms), | |
| | | 13.76(br, 1H) | |
| 82 | 3-(5-Ethyl-7,7-dimethyl- | 1.21, (t, 3H), 1.33(s, 6H), | 480.3(ESI+) |
| | 6-oxo-3,5,6,7- | 3.78(q, 2H), 4.56(d, 2H), | |
| | tetrahydro-imidazo[4,5- | 7.04 and 7.44(s, 1H, two | |
| | f]indol-2-yl)-1H- | tautomeric forms), 7.35(d, | |
| | indazole-5-carboxylic | 2H), 7.46 and 7.78(s, 1H, | |
| | acid (pyridin-4- | two tautomeric forms), | |
| | ylmethyl)-amide; | 7.71(d, 1H), 7.99(d, 1H), | |
| | compound with acetic | 8.52(d, 2H), 9.08(br, 1H), | |
| | acid | 9.27(t, 1H), 13.02 and 13.08(s, | |
| | | 1H, two tautomeric forms), | |
| | | 13.79(br, 1H) | |

### Example 83

### 3-(5-Ethyl-7,7-dimethyl-6-oxo-3,5,6,7-tetrahydro-imidazo[4,5-f]indol-2-yl)-1H-indazole-5-carboxylic acid phenylamide

To a suspension of 3-(5-ethyl-7,7-dimethyl-6-oxo-3,5,6,7-tetrahydro-imidazo[4,5-f]indol-2-yl)-1H-indazole-5-carboxylic acid (example 69, 150 mg, 0.385 mmol) and DMF (7 μl) in THF (9 ml) was added dropwise a solution of oxalyl chloride (195.6 mg, 132 μl 1.54 mmol) in THF (1 ml) at room temperature. After 1 h further 2 equivalents of oxalyl chloride were added. After 2 h reaction was complete. The reaction mixture was added dropwise to a cooled solution (5° C.) of aniline (109.8 mg, 107 μl, 1.15 mmol) and triethylamine (233.8 mg, 321 μl, 2.31 mmol) in THF (5 ml) over 20 minutes. The mixture was allowed to warm to room temperature and reaction was complete after 2 h. The mixture was washed with brine, sodium carbonate solution and again brine. The solvent was evaporated and the residue was purified by silicagel chromatography (ethyl acetate) to yield 148 mg 3-(5-ethyl-7,7-dimethyl-6-oxo-3,5,6,7-tetrahydro-imidazo[4,5-f]indol-2-yl)-1H-indazole-5-carboxylic acid phenylamide (0.312 mmol, 81%)

MS: M=465.2 (ESI+)

¹H-NMR (400 MHz, DMSO): δ (ppm)=1.21 (t, 3H), 1.33 (s, 6H), 3.78 (q, 2H), 7.05 and 7.48 (s, 1H, two tautomeric forms), 7.12 (t, 1H), 7.39 (t, 2H), 7.46 and 7.84 (s, 1H, two tautomeric forms), 7.74 (d, 1H), 7.83 (d, 2H), 8.02 (d, 1H), 9.11 and 9.12 (s, 1H, two tautomeric forms), 10.46 and 10.48 (s, 1H, two tautomeric forms), 13.04 and 13.10 ( s, 1H, two tautomeric forms), 13.80 and 13.84 (s, 1H, two tautomeric forms)

In an analogous manner as described for example 83 the following examples 84-98 were prepared from the appropriate amines:

| Example | | ¹H-NMR(400MHz, | |
|---|---|---|---|
| No | Systematic Name | DMSO): δ(ppm)= | MS: M= |
| 84 | 3-(5-Ethyl-7,7-dimethyl- | 1.18(t, 3H), 1.21(t, 3H), | 417.2(ESI+) |
| | 6-oxo-3,5,6,7- | 1.33(s, 3H), 1.34(s, 3H), | |
| | tetrahydro-imidazo[4,5- | 3.36(q, 2H), 3.79(q, 2H), | |
| | f]indol-2-yl)-1H- | 7.04 and 7.45(s, 1H, two | |
| | indazole-5-carboxylic | tautomeric forms), 7.46 and | |
| | acid ethylamide | 7.79(s, 1H, two tautomeric | |
| | | forms), 7.76(d, 1H), | |
| | | 7.91(d, 1H), 8.59(t, 1H), 8.99 | |
| | | and 9.01(s, 1H, two | |
| | | tautomeric forms), 13.00 | |
| | | and 13.06(s, 1H, two | |
| | | tautomeric forms), 8.99 and | |
| | | 9.01(s, 1H, two tautomeric | |
| | | forms) | |
| 85 | 3-(5-Ethyl-7,7-dimethyl- | 1.21(t, 3H), 1.33(s, 6H), | 515.3(ESI+) |
| | 6-oxo-3,5,6,7- | 3.78(q, 2H), 4.54(d, 2H), | |
| | tetrahydro-imidazo[4,5- | 7.04 and 7.44(s, 1H, two | |
| | f]indol-2-yl)-1H- | tautomeric forms), 7.08(t, | |
| | indazole-5-carboxylic | 1H), 7.24(t, 1H), 7.46 and | |
| | acid 2,4-difluoro- | 7.78(s, 1H, two tautomeric | |
| | benzylamide | forms), 7.47(q, 1H), | |
| | | 7.69(d, 1H), 7.97(d, 1H); 9.05 | |
| | | and 9.07(s, 1H, two | |
| | | tautomeric forms), 9.16(t, | |
| | | 1H), 13.01 and 13.07(s, 1H, | |
| | | two tautomeric forms), | |
| | | 13.75(br, 1H) | |
| 86 | 3-(5-Ethyl-7,7-dimethyl- | 1.21(t, 3H), 1.33(s, 6H), | 563.2(ESI+) |
| | 6-oxo-3,5,6,7- | 3.78(q, 2H), 4.59(d, 2H), | |
| | tetrahydro-imidazo[4,5- | 7.04 and 7.41(s, 1H, two | |
| | f]indol-2-yl)-1H- | tautomeric forms), 7.26(d, | |
| | indazole-5-carboxylic | 1H), 7.36(s, 1H), 7.43(d, | |
| | acid 3-trifluoromethoxy- | 1H), 7.49(m, 1H), 7.51 and | |
| | benzylamide | 7.77(s, 1H, two tautomeric | |
| | | forms), 7.70(d, 1H), | |
| | | 7.97(d, 1H), 9.07(d, 1H), | |
| | | 9.26(t, 1H), 13.01 and 13.07(s, | |
| | | 1H; two tautomeric forms), | |
| | | 13.76(br, 1H) | |
| 87 | 3-(5-Ethyl-7,7-dimethyl- | 1.21(t, 3H), 1.33(s, 6H), | 545.2(ESI+) |
| | 6-oxo-3,5,6,7- | 3.78(q, 2H), 4.52(d, 2H), | |
| | tetrahydro-imidazo[4,5- | 7.04 and 7.38(s, 1H, two | |
| | f]indol-2-yl)-1H- | tautomeric forms), 7.16(d, | |
| | indazole-5-carboxylic | 2H), 7.20(t, 1H), 7.42(d, | |
| | acid 4-difluoromethoxy- | 2H), 7.46 and 7.77(s, 1H, | |
| | benzylamide | two tautomeric forms), | |
| | | 7.68(d, 1H), 7.97(d, 1H), 9.04 | |
| | | and 9.07(s, 1H, two | |
| | | tautomeric forms), 9.19(t, | |
| | | 1H), 13.01 and 13.07(s, 1H, | |
| | | two tautomeric forms), | |
| | | 13.76(br, 1H) | |
| 88 | 3-(5-Ethyl-7,7-dimethyl- | 1.21(t, 3H), 1.33(s, 6H), | 513.3(ESI+) |
| | 6-oxo-3,5,6,7- | 3.78(q, 2H), 4.54(d, 2H), | |
| | tetrahydro-imidazo[4,5- | 7.04 and 7.35(s, 1H, two | |
| | f]indol-2-yl)-1H- | tautomeric forms), | |
| | indazole-5-carboxylic | 7.32-7.46(m, 4H), 7.43 and | |
| | acid 3-chloro- | 7.78(s, 1H, two tautomeric | |
| | benzylamide | forms), 7.69(d, 1H), | |
| | | 7.99(d, 1H), 9.06 and 9.08(s, | |
| | | 1H, two tautomeric forms), | |
| | | 9.23(t, 1H), 13.01 and | |
| | | 13.07(s, 1H, two tautomeric | |
| | | forms), 13.75 and 13.80(s, | |
| | | 1H, two tautomeric forms) | |
| 89 | 5-Ethyl-7,7-dimethyl-2- | 1.21(t, 3H), 1.33(s, 6H), | 457.1(API+) |
| | [5-(piperidine-1- | 1.45-1.70(m, 6H), | |
| | carbonyl)-1H-indazol-3- | 3.4-3.7(m, 4H), 3.78(q, 2H), 7.03 | |
| | yl]-5,7-dihydro-3H- | and 7.42(s, 1H, two | |
| | imidazo[4,5-f]indol-6- | tautomeric forms), 7.45 and | |
| | one | 7.75(s, 1H, two tautomeric | |
| | | forms), 7.47(d, 1H), | |
| | | 7.68(d, 1H), 8.54 and 8.57(s, | |
| | | 1H, two tautomeric | |
| | | forms), 13.00 and 13.06(s, | |
| | | 1H, two tautomeric forms), | |
| | | 13.71(br, 1H) | |
| 90 | 5-Ethyl-7,7-dimethyl-2- | 1.21(t, 3H), 1.33(s, 6H), | 472.3(ESI+) |
| | [5-(4-methyl- | 2.24(s, 3H); 2.39(br, 4H), | |
| | piperazine-1-carbonyl)- | 3.58(br, 4H), 3.78(q, 2H), | |
| | 1H-indazol-3-yl]-5,7- | 7.04 and 7.41(s, 1H, two | |
| | dihydro-3H- | tautomeric forms), 7.45 and | |
| | imidazol[4,5-f]indol-6- | 7.75(s, 1H, two tautomeric | |
| | one | forms), 7.48(d, 1H), | |
| | | 7.69(d, 1H), 8.57 and 8.60(s, | |
| | | 1H, two tautomeric | |
| | | forms), 13.00 and 13.06(s, | |
| | | 1H, two tautomeric forms), | |
| | | 13.74 and 13.77(s, 1H, two | |
| | | tautomeric forms) | |
| 91 | 5-Ethyl-7,7-dimethyl-2- | 1.21(t, 3H), 1.33(s, 6H), | 459.3(ESI+) |
| | [5-(morpholine-4- | 3.4-3.7(m, 8H), 3.79(q, | |
| | carbonyl)-1H-indazol-3- | 2H), 7.03 and 7.41(s, 1H, | |
| | yl]-5,7-dihydro-3H- | two tautomeric forms), 7.45 | |
| | imidazol[4,5-f]indol-6- | and 7.76(s, 1H, two | |
| | one | tautomeric forms), 7.51(d, | |
| | | 1H), 7.69(d, 1H), 8.59 and | |
| | | 8.62(s, 1H, two tautomeric | |
| | | forms), 13.00 and 13.06(s, | |
| | | 1H, two tautomeric forms), | |
| | | 13.73(br, 1H) | |
| 92 | 2-[5-(4-Acetyl- | 1.21(t, 3H), 1.33(s, 6H), | 500.4(ESI+) |
| | piperazine-1-carbonyl)- | 2.04(s, 3H), 3.4-3.7(m, | |
| | 1H-indazol-3-yl]-5- | 8H), 3.78(q, 2H), 7.04 and | |
| | ethyl-7,7-dimethyl-5,7- | 7.41(s, 1H, two tautomeric | |
| | dihydro-3H- | forms), 7.45 and 7.75(s, 1H, | |
| | imidazo[4,5-f]indol-6- | two tautomeric forms), | |
| | one | 7.53(d, 1H), 7.70(d, 1H), 8.61 | |
| | | and 8.63(s, 1H, two | |
| | | tautomeric forms), 13.02 and | |
| | | 13.08(s, 1H, two tautomeric | |
| | | forms), 13.74 and 13.78(s, | |
| | | 1H, two tautomeric forms) | |
| 93 | 3-(5-Ethyl-7,7-dimethyl- | 1.21(t, 3H), 1.33(s, 6H), | 563.4(ESI+) |
| | 6-oxo-3,5,6,7- | 3.78(q, 2H), 4.59(d, 2H), | |
| | tetrahydro-imidazo[4,5- | 7.04 and 7.44(s, 1H, two | |
| | f]indol-2-yl)-1H- | tautomeric forms), 7.35(d, | |
| | indazole-5-carboxylic | 2H), 7.46 and 7.77(s, 1H, | |
| | acid 4-trifluoromethoxy- | two tautomeric forms), | |
| | benzylamide | 7.50(d, 2H), 7.70(d, 1H), | |
| | | 7.98(d, 1H), 9.06 and 9.08(s, | |
| | | 1H, two tautomeric forms), | |
| | | 9.24(t, 1H), 13.01 and | |
| | | 13.08(s, 1H, two tautomeric | |
| | | forms), 13.76(br, 1H) | |
| 94 | 5-Ethyl-2-[5-(4- | 1.00(d, 6H), 1.21(t, 3H), | 500.4(ESI+) |
| | isopropyl-piperazine-1- | 1.33(s, 6H), 2.72(br, 4H), | |
| | carbonyl)-1H-indazol-3- | 3.60(br, 4H), 3.78(q, 2H), | |
| | yl]-7,7-dimethyl-5,7- | 7.04 and 7.40(s, 1H, two | |
| | dihydro-3H- | tautomeric forms), 7.45 and | |
| | imidazo[4,5-f]indol-6- | 7.77(s, 1H, two tautomeric | |
| | one; compound with | forms), 7.48(d, 1H), | |
| | acetic acid | 7.68(d, 1H), 8.58 and 8.61(s, | |
| | | 1H, two tautomeric forms), | |
| | | 13.01 and 13.07(s, 1H, two | |
| | | tautomeric forms), 13.72 | |
| | | and 13.76(s, 1H, two | |
| | | tautomeric forms) | |
| 95 | 5-Ethyl-7,7-dimethyl-2- | 1.21(t, 3H), 1.33(s, 6H), | 475.3(ESI+) |
| | [5-(thiomorpholine-4- | 2.67(br, 4H), 3.78(m, 6H), | |
| | carbonyl)-1H-indazol-3- | 7.04 and 7.41(s, 1H, two | |
| | yl]-5,7-dihydro-3H- | tautomeric forms), 7.45 and | |
| | imidazo[4,5-f]indol-6- | 7.75(s, 1H, two tautomeric | |
| | one | forms), 7.49(d, 1H), | |
| | | 7.69(d, 1H), 8.55 and 8.58(s, | |
| | | 1H, two tautomeric forms), | |
| | | 13.01 and 13.07(s, 1H, two | |
| | | tautomeric forms), | |
| | | 13.73(br, 1H) | |
| 96 | 5-Ethyl-7,7-dimethyl-2- | 1.21(t, 3H), 1.33(s, 6H), | 461.3(ESI+) |
| | [5-(thiazolidine-3- | 3.07(br, 2H), 3.78(br, 2H), | |
| | carbonyl)-1H-indazol-3- | 3.86(br, 2H), 4.67(br, 2H), | |
| | yl]-5,7-dihydro-3H- | 7.04 and 7.43(s, 1H, two | |
| | imidazo[4,5-f]indol-6- | tautomeric forms), 7.45 and | |
| | one | 7.77(s, 1H, two tautomeric | |
| | | forms), 7.64(d, 1H), | |
| | | 7.70(d, 1H), 8.72 and 8.74(s, | |
| | | 1H, two tautomeric forms), | |
| | | 13.03 and 13.09(s, 1H, two | |
| | | tautomeric forms), 13.77(br, | |
| | | 1H) | |
| 97 | 5-Ethyl-2-[5-(4- | 1.21(t, 3H), 1.33(s, 6H), | 536.5(ESI+) |
| | methanesulfonyl- | 2.92(s, 3H), 3.19(br, 4H), | |
| | piperazine-1-carbonyl)- | 3.68(br, 4H), 3.78(q, 2H), | |
| | 1H-indazol-3-yl]-7,7- | 7.04 and 7.39(s, 1H, two | |
| | dimethyl-5,7-dihydro- | tautomeric forms), 7.45 and | |
| | 3H-imidazo[4,5-f]indol- | 7.73(s, 1H, two tautomeric | |
| | 6-one | forms), 7.52(d, 1H), | |
| | | 7.70(d, 1H), 8.61 and 8.64(s, | |
| | | 1H, two tautomeric forms), | |
| | | 13.02 and 13.08(s, 1H, two | |
| | | tautomeric forms), 13.75 | |
| | | and 13.78(s, 1H, two | |
| | | tautomeric forms) | |
| 98 | 2-[5-(1,1-Dioxo-1λ⁶- | 1.21(t, 3H), 1.33(s, 6H), | 507.4(ESI+) |
| | thiomorpholine-4- | 3.29(br, 4H), 3.78(q, 2H), | |
| | carbonyl)-1H-indazol-3- | 3.95(br, 4H), 7.04 and | |
| | yl]-5-ethyl-7,7- | 7.37(s, 1H, two tautomeric | |
| | dimethyl-5,7-dihydro- | forms), 7.46 and 7.72(s, | |
| | 3H-imidazo[4,5-f]indol- | 1H, two tautomeric forms), | |
| | 6-one | 7.60(d, 1H), 7.69(d, 1H), | |
| | | 8.66 and 8.68(s, 1H, two | |
| | | tautomeric forms), 13.01 | |
| | | and 13.08(s, 1H, two | |
| | | tautomeric forms), 13.75 | |
| | | and 13.79(s, 1H, two | |
| | | tautomeric forms) | |

### Example 99

### 5-Ethyl-7,7-dimethyl-2-[5-(1-oxo-1λ⁴-thiomorpholine-4-carbonyl)-1H-indazol-3-yl]-5,7-dihydro-3H-imidazo[4,5-f]indol-6-one

5-Ethyl-7,7-dimethyl-2-[5-(1-oxo-1λ⁴-thiomorpholine-4-carbonyl)-1H-indazol-3-yl]-5,7-dihydro-3H-imidazo[4,5-f]indol-6-one was isolated as a byproduct during formation of 5-ethyl-7,7-dimethyl-2-[5-(thiomorpholine-4-carbonyl)-1H-indazol-3-yl]-5,7-dihydro-3H-imidazo[4,5-f]indol-6-one (example 95).

MS: M=491.2 (ESI+)

¹H-NMR (400 MHz, DMSO): δ (ppm)=1.21 (t, 3H), 1.33 (s, 6H), 3.50-3.60 (m, 4H), 3.70-3.85 (m, 6H), 7.03 and 7.39 (s, 1H, two tautomeric forms), 7.45 and 7.74 (s, 1H, two tautomeric forms), 7.55 (d, 1H), 7.71 (d, 1H), 8.64 (br, 1H), 13.01 and 13.07 (s, 1H, two tautomeric forms), 13.75 and 13.79 (s, 1H, two tautomeric forms)

### Example 100

### 3-(5-Ethyl-7,7-dimethyl-6-oxo-3,5,6,7-tetrahydro-imidazo[4,5-f]indol-2-yl)-1H-indazole-5-carboxylic acid amide

To a suspension of 3-(5-ethyl-7,7-dimethyl-6-oxo-3,5,6,7-tetrahydro-imidazo[4,5-f]indol-2-yl)-1H-indazole-5-carboxylic acid (example 69, 500 mg, 1.28 mmol) and DMF (1 drop) in THF (15 ml) at 0° C. under a nitrogen atmosphere was added oxalyl chloride (494 mg, 335 μl, 3.89 mmol). The mixture was allowed to warm to room temperature and stirred for 5.5 h. After 3 and 4 h additional 1 and 0.5 equivalents of oxalyl chloride were added. The reaction mixture was added to an aqueous solution of ammonia (25%, 250 ml, 3339 mmol) stirred for 1 h at room temperature. The aqueous phase was extracted three times with ethyl acetate and the solvent of the combined organic phases was evaporated. The residue was triturated with diisopropyl ether/n-heptane and with water and then dried in vacuum. 410 mg 3-(5-ethyl-7,7-dimethyl-6-oxo-3,5,6,7-tetrahydro-imidazo[4,5-f]indol-2-yl)-1H-indazole-5-carboxylic acid amide (1.056 mmol, 82%) were obtained.

MS: M=389.2 (ESI+)

¹H-NMR (400 MHz, DMSO): δ (ppm)=1.22 (t, 3H), 1.36 (s, 6H), 3.81 (q, 2H), 7.28 (br, 1H), 7.41 (br, 1H), 7.68 (br, 1H), 7.71 (m, 1H), 7.99 (m,1H), 8.09 (br, 1H), 9.10 (s, 1H), 14.04 (br, 1H)

### Example 101

### 3-(5-Ethyl-7,7-dimethyl-6-oxo-3,5,6,7-tetrahydro-imidazo[4,5-f]indol-2-yl)-1H-indazole-5-carboxylic acid methyl ester

To a suspension of 3-(5-ethyl-7,7-dimethyl-6-oxo-3,5,6,7-tetrahydro-imidazo[4,5-f]indol-2-yl)-1H-indazole-5-carboxylic acid (example 69, 200 mg, 0.513 mmol) and DMF (9 μl) in THF (20 ml) was added dropwise a solution of oxalyl chloride (260.6 mg, 176 μl, 2.05 mmol) in THF (2 ml) at room temperature. After 1 h reaction was complete. The reaction mixture was cooled to 5° C. and a mixture of methanol (329 mg, 416 μl, 10.27 mmol) and triethylamine (260 mg, 358 μl, 2.56 mmol) was added dropwise. The reaction mixture was warmed to 30° C. After 1 h the solvent was evaporated and the residue dissolved in ethyl acetate. The organic phase was washed with bicarbonate solution and three times with water. The solvent was evaporated and the residue was dried in vacuum to yield 213 mg 3-(5-ethyl-7,7-dimethyl-6-oxo-3,5,6,7-tetrahydro-imidazo[4,5-f]indol-2-yl)-1H-indazole-5-carboxylic acid methyl ester (0.507 mmol, 99%)

MS: M=404.1 (ESI+)

¹H-NMR (400 MHz, DMSO): δ (ppm)=1.21 (t, 3H), 1.34 (s, 6H), 3.80 (q, 2H), 3.94 (s, 3H), 7.05 and 7.47 (s, 1H, two tautomeric forms), 7.50 and 7.84 (s, 1H, two tautomeric forms), 7.74 (d, 1H), 8.04 (d, 1H), 9.22 and 9.24 (s, 1H, two tautomeric forms), 13.06 and 13.12 (s, 1H, two tautomeric forms), 13. 87 and 13.91 (s, 1H, two tautomeric forms)

### Example 102

### 3-(5-Ethyl-7,7-dimethyl-6-oxo-3,5,6,7-tetrahydro-imidazo[4,5-f]indol-2-yl)-1H-indazole-5-carboxylic acid methoxy-methyl-amide

3-(5-Ethyl-7,7-dimethyl-6-oxo-3,5,6,7-tetrahydro-imidazo[4,5-f]indol-2-yl)-1H-indazole-5-carboxylic acid methoxy-methyl-amide was prepared in an analogous manner as described for example 83 from N,O-dimethylhydroxylamine hydrochloride as amine instead of aniline and pyridine as base instead of triethylamine.

MS: M=433.1 (API+)

¹H-NMR (400 MHz, DMSO): δ (ppm)=1.21 (t, 3H), 1.33 (s, 6H), 3.24 and 3.34 (s, 3H, two tautomeric forms), 3.58 and 3.59 (s, 3H, two tautomeric forms), 3.78 (q, 2H), 7.04 and 7.44 (s, 1H, two tautomeric forms), 7.46 and 7.78 (s, 1H, two tautomeric forms), 7.67-7.73 (m, 2H), 8.86 and 8.87 (s, 1H, two tautomeric forms), 13.02 and 13.08 (s, 1H, two tautomeric forms), 13.74 and 13.78 (s, 1H, two tautomeric forms)

### Example 103

### 2-(5-Acetyl-1H-indazol-3-yl)-5-ethyl-7,7-dimethyl-5,7-dihydro-3H-imidazo[4,5-f]indol-6-one

To a suspension of 3-(5-ethyl-7,7-dimethyl-6-oxo-3,5,6,7-tetrahydro-imidazo[4,5-f]indol-2-yl)-1H-indazole-5-carboxylic acid methoxy-methyl-amide (example 102, 100 mg, 0.231 mmol) in THF (5 ml) under a nitrogen atmosphere at 0° C. was added methylmagnesium iodide (3M in diethylether, 231 μl, 0.694 mmol). After 1.5 h at 5° C. additional 3 equivalents of methylmagnesium iodide were added and the mixture was allowed to warm to room temperature. After 12 h the mixture was poured into water (9 ml)/acetic acid solution (25%, 1 ml). The organic phase was separated and washed with bicarbonate solution. The aqueous phases were washed with ethyl acetate, the combined organic phases washed with water and dried over MgSO₄. The solvent was evaporated and the residue purified by silica gel chromatography (dichloromethane/methanol, 98:2→95:5) to yield 32 mg 2-(5-acetyl-1H-indazol-3-yl)-5-ethyl-7,7-dimethyl-5,7-dihydro-3H-imidazo[4,5-f]indol-6-one (0.08 mmol, 35%)

MS: M=388.2 (ESI+)

¹H-NMR (400 MHz, DMSO): δ (ppm)=1.21 (t, 3H), 1.34 (s, 6H), 2.71 (s, 3H), 3.78 (q, 2H), 7.04 and 7.47 (s, 1H, two tautomeric forms), 7.47 and 7.82 (s, 1H, two tautomeric forms), 7.72 (d, 1H), 8.05 (d, 1H), 9.18 (s, 1H), 13.06 (br, 1H), 13.86 (br, 1H)

### Example 104

### 3-(7,7-Dimethyl-6-oxo-3,5,6,7-tetrahydro-imidazo[4,5-f]indol-2-yl)-1H-indazole-6-carboxylic acid benzylamide

3-(7,7-Dimethyl-6-oxo-3,5,6,7-tetrahydro-imidazo[4,5-f]indol-2-yl)-1H-indazole-6-carboxylic acid benzylamide was prepared in an analogous manner as described for example 76ii from 3-(7,7-dimethyl-6-oxo-3,5,6,7-tetrahydro-imidazo[4,5-f]indol-2-yl)-1H-indazole-6-carboxylic acid and benzyl amine. 3-(7,7-Dimethyl-6-oxo-3,5,6,7-tetrahydro-imidazo[4,5-f]indol-2-yl)-1H-indazole-6-carboxylic acid was prepared in an analogous manner as described for example 69 from indole-6-carboxylic acid and 5,6-diamino-3,3-dimethyl-1,3-dihydro-indol-2-one (U.S. Pat. No. 4,666,923A) and was used without further purification.

MS: M=451.2 (ESI+)

¹H-NMR (400 MHz, DMSO): δ (ppm)=1.32 (s, 6H), 4.54 (d, 2H), 6.94 and 7.11 (s, 1H, two tautomeric forms), 7.25 (m, 1H), 7.33-7.36 (m, 4H), 7.39 and 7.66 (s, 1H, two tautomeric forms), 7.82 (d, 1H), 8.17 (s, 1H), 8.53 (m, 1H), 9.24 (m, 1H), 10.28 and 10.32 (br, 1H, two tautomeric forms), 12.83 and 12.98 (br, 1H, two tautomeric forms)

In an analogous manner as described for example 104 the following examples 105-106 were prepared from the appropriate amines:

| Example | | ¹H-NMR(400MHz, | |
|---|---|---|---|
| No | Systematic Name | DMSO): δ(ppm)= | MS: M= |
| 105 | 3-(7,7-Dimethyl-6-oxo- | 1.17(t, 3H), 1.32(s, 6H), | 389.1(ESI+) |
| | 3,5,6,7-tetrahydro- | 3.34(m, 2H), 6.94 and | |
| | imidazo[4,5-f]indol-2- | 7.11(s, 1H, two tautomeric | |
| | yl)-1H-indazole-6- | forms), 7.38 and 7.65(s, 1H, | |
| | carboxylic acid ethylamide | two tautomeric forms), | |
| | | 7.76(d, 1H), 8.10(s, 1H), | |
| | | 8.50(m, 1H), 8.65(s, 1H), 10.28 | |
| | | and 10.32(br, 1H, two | |
| | | tautomeric forms), 12.81 | |
| | | and 12.97(br, 1H, two | |
| | | tautomeric forms), | |
| | | 13.77(br, 1H) | |
| 106 | 3-(7,7-Dimethyl-6-oxo- | 1.33(s, 6H), 6.95 and | 437.2(ESI+) |
| | 3,5,6,7-tetrahydro- | 7.13(s, 1H, two tautomeric | |
| | imidazo[4,5-f]indol-2- | forms), 7.13(t, 1H), | |
| | yl)-1H-indazole-6- | 7.38(m, 2H), 7.38 and 7.67(s, | |
| | carboxylic acid | 1H, two tautomeric forms), | |
| | phenylamide | 7.81-7.87(m, 3H), 8.24(s, | |
| | | 1H), 8.57(d, 1H), 10.32(br, | |
| | | 1H), 10.45(br, 1H), | |

In an analogous manner as described for example 79 the following examples 107-109 were prepared from 3-(5-Ethyl-7,7-dimethyl-6-oxo-3,5,6,7-tetrahydro-imidazo[4,5-f]indol-2-yl)-1H-indazole-6-carboxylic acid (example 74) and the appropriate amines:

| Example | | ¹H-NMR(400MHz, | |
|---|---|---|---|
| No | Systematic Name | DMSO): δ(ppm)= | MS: M= |
| 107 | 3-(5-Ethyl-7,7-dimethyl- | 1.17(t, 3H), 1.23(m, 3H), | 417.3(ESI+) |
| | 6-oxo-3,5,6,7- | 1.33(s, 3H), 1.34(s, 3H), | |
| | tetrahydro-imidazo[4,5- | 3.37(m, 2H), 3.79(m, 2H), | |
| | f]indol-2-yl)-1H- | 7.04 and 7.39(s, 1H, two | |
| | indazole-6-carboxylic | tautomeric forms), 7.45 and | |
| | acid ethylamide | 7.74(s, 1H, two tautomeric | |
| | | forms), 7.77(m, 1H), | |
| | | 8.11(s, 1H), 8.51(m, 1H), | |
| | | 8.66(m, 1H), 12.98 | |
| | | and 13.04(br, 1H, two tautomeric | |
| | | forms), 13.79 and 13.83(br, | |
| | | 1H, two tautomeric forms) | |
| 108 | 3-(5-Ethyl-7,7-dimethyl- | 1.22(t, 3H), 1.33(s, 3H), | 465.3(ESI+) |
| | 6-oxo-3,5,6,7- | 1.34(s, 3H), 3.80(m, 2H), | |
| | tetrahydro-imidazo[4,5- | 7.05 and 7.37(s, 1H, two | |
| | f]indol-2-yl)-1H- | tautomeric forms), 7.13(t, | |
| | indazole-6-carboxylic | 1H), 7.39(s, 1H), 7.41(d, | |
| | acid phenylamide | 1H), 7.46 and 7.76(s, 1H, | |
| | | two tautomeric forms), | |
| | | 7.81(s, 1H), 7.84(s, 1H), | |
| | | 7.87(m, 1H), 8.24(s, 1H), | |
| | | 8.59(m, 1H), 10.45(s, 1H), | |
| | | 13.01 and 13.07(br, 1H, two | |
| | | tautomeric forms), 13.89 | |
| | | and 13.93(br, 1H, two | |
| | | tautomeric forms) | |
| 109 | 3-(5-Ethyl-7,7-dimethyl- | 1.21(m, 3H), 1.33(s, 3H), | 479.3(ESI+) |
| | 6-oxo-3,5,6,7- | 1.34(s, 3H), 3.79(m, 2H), | |
| | tetrahydro-imidazo[4,5- | 4.54(d, 2H), 7.04 and | |
| | f]indol-2-yl)-1H- | 7.33(s, 1H, two tautomeric | |
| | indazole-6-carboxylic | forms), 7.26(m, 1H), | |
| | acid benzylamide | 7.35-7.40(m, 4H), 7.45 and | |
| | | 7.74(s, 1H, two tautomeric | |
| | | forms), 7.83(m, 1H), | |
| | | 8.18(m, 1H), 8.54(m, 1H), | |
| | | 9.24(m, 1H), 12.99 and | |
| | | 13.05(br, 1H, two tautomeric | |
| | | forms), 13.82 and 13.86(br, | |
| | | 1H, two tautomeric forms) | |

In an analogous manner as described for example 100 the following example 110 was prepared from 3-(5-ethyl-7,7-dimethyl-6-oxo-3,5,6,7-tetrahydro-imidazo[4,5-f]indol-2-yl)-1H-indazole-6-carboxylic acid (example 74):

| Example | | ¹H-NMR(400MHz, | |
|---|---|---|---|
| No | Systematic Name | DMSO): δ(ppm)= | MS: M= |
| 110 | 3-(5-Ethyl-7,7- | 1.21(t, 3H), 1.34(s, 6H), | 389.2(ESI+) |
| | dimethyl- | 3.79(m, 2H), 7.04 and | |
| | 6-oxo-3,5,6,7- | 7.39(s, 1H, two tautomeric | |
| | tetrahydro- | forms), 7.47 and | |
| | imidazo[4,5- | 7.74(s, 1H, | |
| | f]indol-2-yl)- | two tautomeric forms), | |
| | 1H-indazole-6- | 7.47(s, 1H), 7.81(d, 1H), | |
| | carboxylic | 8.16(m, 2H), | |
| | acid amide | 8.51(d, 1H), 12.99 | |
| | | and 13.05(br, 1H, two | |
| | | tautomeric forms), | |
| | | 13.83(br, 1H) | |

### Example 111

### 2-(5-Amino-1H-indazol-3-yl)-5-ethyl-7,7-dimethyl-5,7-dihydro-3H-imidazo[4,5-f]indol-6-one

5-Ethyl-7,7-dimethyl-2-(5-nitro-1H-indazol-3-yl)-5,7-dihydro-3H-imidazo[4,5-f]indol-6-one (example 71, 3.9 g, 9.99 mmol) was hydrogenated in methanol (300 ml) and THF (300 ml) over Raney/Nickel at 30 mbar for 8 h. The catalyst was filtered off and washed with methanol. The solvent was evaporated to yield 3.4 g 2-(5-amino-1H-indazol-3-yl)-5-ethyl-7,7-dimethyl-5,7-dihydro-3H-imidazo[4,5-f]indol-6-one (9.43 mmol, 94%)

MS: M=361.1 (ESI+)

¹H-NMR (400 MHz, DMSO): δ (ppm)=1.21 (m, 3H), 1.33 (m, 6H), 3.78 (m, 2H), 5.05 (s, 2H), 6.87 (m, 1H), 7.00 and 7.28 (s, 1H), 7.33 (m, 1H), 7.40 and 7.64 (s, 1H), 7.56 (d, 1H), 12.70 and 12.76 (s, 1H), 13.08 and 13.13 (s, 1H)

### Example 112

### 2-(5-Amino-1H-indazol-3-yl)-5-isopropyl-7,7-dimethyl-5,7-dihydro-1H-imidazo[4,5-f]indol-6-one

5-Isopropyl-7,7-dimethyl-2-(5-nitro-1H-indazol-3-yl)-5,7-dihydro-1H-imidazo[4,5-f]indol-6-one (1.9 g, 4.69 mmol; obtained in an analogous manner as described in example 71 from 5,6-diamino-1-isopropyl-3,3-dimethyl-1,3-dihydro-indol-2-one and 5-nitro-1H-indazole-3-carbaldehyde) was hydrogenated in methanol (25 ml) and THF (25 ml) over Pd/C (2g) for 2h. The catalyst was filtered off and washed with methanol. The solvent was evaporated to yield 1.43 g 2-(5-amino-1H-indazol-3-yl)-5-isopropyl-7,7-dimethyl-5,7-dihydro-1H-imidazo[4,5-f]indol-6-one (3.82 mmol, 81%)

MS: M=375.29 (ESI+)

¹H-NMR (400 MHz, DMSO): δ (ppm)=1.31 (s, 6H), 1.47 (m, 6H), 4.59 (m, 1H), 6.93 (d, 1H), 7.23 (bs, 1H), 7.38 (d, 1H), 7.53 (bs, 1H), 7.66 (s, 1H), 13.20 (s, 1H)

### Example 113

### N-[3-(5-Ethyl-7,7-dimethyl-6-oxo-1,5,6,7-tetrahydro-imidazo[4,5-f]indol-2-yl)-1H-indazol-5-yl]-2-o-tolyl-acetamide

To a solution of o-tolylacetic acid (83 mg, 0.610 mmol) in absolute DMF (3 ml) under a nitrogen atmosphere were added N′-(3-dimethylaminopropyl)-N-ethylcarbodiimide hydrochloride (128 mg, 0.668 mmol) and hydroxybenzotriazole hydrate (102 mg, 0.666 mmol). After 90 minutes at room temperature 2-(5-amino-1H-indazol-3-yl)-5-ethyl-7,7-dimethyl-5,7-dihydro-3H-imidazo[4,5-f]indol-6-one (example 111, 200 mg, 0.555 mmol) was added and stirring continued for 4h. The reaction mixture was treated with water (35 ml) and the aqueous phase extracted twice with ethyl acetate (2×50 ml). The combined organic phases were washed with bicarbonate solution and brine, dried over MgSO₄ and the solvent was evaporated. The residue was purified by HPLC chromatography to yield 170 mg N-[3-(5-ethyl-7,7-dimethyl-6-oxo-1,5,6,7-tetrahydro-imidazo[4,5-f]indol-2-yl)-1H-indazol-5-yl]-2-o-tolyl-acetamide (0.345 mmol, 62%).

MS: M=491.4 (ESI−)

¹H-NMR (400 MHz, DMSO): δ (ppm)=1.29 (m, 3H), 1.33 (s, 6H), 2.36 (s, 3H), 3.74 (s, 2H), 3.78 (m, 2H), 7.02 and 7.36 (s, 1H, two tautomeric forms), 7.18 (m, 3H), 7.31 (t, 1H), 7.43 and 7.71 (s, 1H, two tautomeric forms), 7.59 (m, 1H), 7.68 (t, 1H), 8.78 (d, 1H), 10.31 (s, 1H), 12.87 and 12.92 (br, 1H, two tautomeric forms), 13.47 and 13.51 (br, 1H, two tautomeric forms)

In an analogous manner as described for example 113 the following examples 114-120 were prepared from the appropriate carboxylic acids:

| Example | | ¹H-NMR(400MHz, | |
|---|---|---|---|
| No | Systematic Name | DMSO): δ(ppm)= | MS: M= |
| 114 | N-[3-(5-Ethyl-7,7- | 1.20(t, 3H), 1.33(s, 6H), | 479.0(API+) |
| | dimethyl-6-oxo-3,5,6,7- | 3.69(s, 2H), 3.78(m, 2H), | |
| | tetrahydro-imidazo[4,5- | 7.02 and 7.39(s, 1H, two | |
| | f]indol-2-yl)-1H- | tautomeric forms), 7.27(m, | |
| | indazol-5-yl]-2-phenyl- | 1H), 7.34-7.40(m, 4H), 7.43 | |
| | acetamide | and 7.71(s, 1H, two | |
| | | tautomeric forms), 7.58(m, | |
| | | 1H), 7.69(m, 1H), 8.76(d, | |
| | | 1H), 10.34(s, 1H), 12.87 | |
| | | and 12.93(br, 1H, two | |
| | | tautomeric forms), 13.47 | |
| | | and 13.51(br, 1H, two | |
| | | tautomeric forms) | |
| 115 | N-[3-(5-Ethyl-7,7- | 1.21(t, 3H), 1.34(s, 6H), | 466.2(ESI+) |
| | dimethyl-6-oxo-1,5,6,7- | 3.78(m, 2H), 7.17(s, 1H), | |
| | tetrahydro-imidazo[4,5- | 7.56(s, 1H), 7.67(d, 1H), | |
| | f]indol-2-yl)-1H- | 7.77(d, 1H), 7.95(m, 1H), | |
| | indazol-5-yl]- | 7.97(m, 1H), 8.81(d, 1H), | |
| | isonicotinamide | 8.82(d, 1H), 8.88(s, 1H) | |
| 116 | Pyridine-2-carboxylic | 1.21(t, 3H), 1.34(s, 6H), | 466.1(ESI+) |
| | acid [3-(5-ethyl-7,7- | 3.79(m, 2H), 7.20(br, 1H), | |
| | dimethyl-6-oxo-1,5,6,7- | 7.60(br, 1H), 7.64(d, 1H), | |
| | tetrahydro-imidazo[4,5- | 7.70(m, 1H), 7.82(m, 1H), | |
| | f]indol-2-yl)-1H- | 8.11(m, 1H), 8.22(d, 1H), | |
| | indazol-5-yl]-amide | 8.77(m, 1H), 9.10(s, 1H), | |
| | | 10.73(br, 1H) | |
| 117 | N-[3-(5-Ethyl-7,7- | 1.21(t, 3H), 1.33(s, 3H), | 491.2(ESI−) |
| | dimethyl-6-oxo-1,5,6,7- | 1.34(s, 3H), 2.29(s, 3H), | |
| | tetrahydro-imidazo[4,5- | 3.64(s, 2H), 3.79(m, 2H), | |
| | f]indol-2-yl)-1H- | 7.02 and 7.36(s, 1H, two | |
| | indazol-5-yl]-2-p-tolyl- | tautomeric forms), 7.16(d, | |
| | acetamide | 2H), 7.28(d, 2H), 7.43 and | |
| | | 7.71(s, 1H, two tautomeric | |
| | | forms), 7.58(m, 1H), | |
| | | 7.68(m, 1H), 8.75(m, 1H), | |
| | | 10.29(br, 1H), 12.87 and | |
| | | 12.93(br, 1H, two | |
| | | tautomeric forms), 13.46 | |
| | | and 13.51(br, 1H, two | |
| | | tautomeric forms) | |
| 118 | 2-(3,5-Dimethoxy- | 1.21(m, 3H), 1.33(s, 3H), | 539.3(ESI+) |
| | phenyl)-N-[3-(5-ethyl- | 1.34(s, 3H), 3.61(s, 2H), | |
| | 7,7-dimethyl-6-oxo- | 3.75(s, 6H), 3.79(m, 2H), | |
| | 1,5,6,7-tetrahydro- | 6.41(s, 1H), 6.57(d, 2H), | |
| | imidazo[4,5-f]indol-2- | 7.02 and 7.36(s, 1H, two | |
| | yl)-1H-indazol-5-yl]- | tautomeric forms), 7.43 and | |
| | acetamide | 7.72(s, 1H, two tautomeric | |
| | | forms), 7.58(m, 1H), | |
| | | 7.69(m, 1H), 8.76(d, 1H), | |
| | | 10.28(s, 1H), 12.87 and 12.93(br, | |
| | | 1H, two tautomeric forms), | |
| | | 13.47 and 13.51(br, 1H, two | |
| | | tautomeric forms) | |
| 119 | N-[3-(5-Ethyl-7,7- | 1.21(t, 3H), 1.33(s, 3H), | 483.1(ESI+) |
| | dimethyl-6-oxo-1,5,6,7- | 1.34(s, 3H), 3.79(m, 2H), | |
| | tetrahydro-imidazo[4,5- | 7.04 and 7.36(s, 1H, | |
| | f]indol-2-yl)-1H- | tautomeric forms), 7.40(m, | |
| | indazol-5-yl]-4-fluoro- | 2H), 7.45 and 7.71(s, 1H, | |
| | benzamide | two tautomeric forms), | |
| | | 7.64(m, 1H), 7.85(m, 1H), | |
| | | 8.14(m, 2H), 8.91(d, 1H), | |
| | | 10.46(s, 1H), 12.91 and 12.97(br, | |
| | | 1H, two tautomeric forms), | |
| | | 13.53 and 13.57(br, 1H, two | |
| | | tautomeric forms) | |
| 120 | N-[3-(5-Ethyl-7,7- | 1.21(t, 3H), 1.33(s, 3H), | 497.1(ESI+) |
| | dimethyl-6-oxo-1,5,6,7- | 1.34(s, 3H), 3.75(s, 2H), | |
| | tetrahydro-imidazo[4,5- | 3.79(m, 2H), 7.02 and | |
| | f]indol-2-yl)-1H- | 7.35(s, 1H, two tautomeric | |
| | indazol-5-yl]-2-(4- | forms), 7.18(t, 2H), 7.42 | |
| | fluoro-phenyl)- | and 7.71(s, 1H, two | |
| | acetamide | tautomeric forms), 7.43(m, | |
| | | 2H), 7.58(m, 1H), 7.69(m, | |
| | | 1H), 8.76(d, 1H), 10.32(s, | |
| | | 1H), 12.86 and 12.92(br, | |
| | | 1H, two tautomeric forms), | |
| | | 13.46 and 13.51(br, 1H, two | |
| | | tautomeric forms) | |

### Example 121

### N-[3-(5-Ethyl-7,7-dimethyl-6-oxo-1,5,6,7-tetrahydro-imidazo[4,5-f]indol-2-yl)-1H-indazol-5-yl]-nicotinamide

To a solution of 2-(5-amino-1H-indazol-3-yl)-5-ethyl-7,7-dimethyl-5,7-dihydro-3H-imidazo[4,5-f]indol-6-one (example 111, 150 mg, 0.416 mmol) in absolute THF (2 ml) and absolute DMF (0.2 ml) at 0° C. were added nicotinyl chloride hydrochloride (65 mg, 0.459 mmol) and diisopropylethylamine (134 mg, 1.04 mmol) under a nitrogen atmosphere. After 5 h at room temperature the reaction mixture was treated with KOH (1M solution, 0.4 ml). After 15 minutes at room temperature the solvent was evaporated and the residue purified by HPLC chromatography to yield 115 mg N-[3-(5-Ethyl-7,7-dimethyl-6-oxo-1,5,6,7-tetrahydro-imidazo[4,5-f]indol-2-yl)-1H-indazol-5-yl]-nicotinamide (0.247 mmol, 59%).

MS: M=466.1 (ESI+)

¹H-NMR (400 MHz, DMSO): δ (ppm)=1.21 (t, 3H), 1.33 (s, 3H), 1.34 (s, 3H), 3.79 (m, 2H), 7.04 and 7.35 (s, 1H, tautomeric forms), 7.45 and 7.71 (s, 1H, two tautomeric forms), 7.59-7.67 (m, 2H), 7.85 (m, 1H), 8.39 (d, 1H), 8.79 (m, 1H), 8.94 (m, 1H), 9.20 (s, 1H), 10.64 (br, 1H), 12.92 and 12.98 (br, 1H, two tautomeric forms), 13.55 and 13.59 (br, 1H, two tautomeric forms)

In an analogous manner as described for example 121 the following examples 122-141 were prepared from the appropriate acyl chlorides, carbamoyl chlorides and sulfonyl chlorides:

| Example | | ¹H-NMR(400MHz, | |
|---|---|---|---|
| No | Systematic Name | DMSO): δ(ppm)= | MS: M= |
| 122 | N-[3-(5-Ethyl-7,7- | 1.14(t, 3H), 1.21(m, 3H), | 417.2(ESI+) |
| | dimethyl-6-oxo-1,5,6,7- | 1.33(s, 6H), 2.36(m, 2H), | |
| | tetrahydro-imidazo[4,5- | 3.78(m, 2H), 7.02 and | |
| | f]indol-2-yl)-1H- | 7.35(s, 1H), 7.43 and 7.65(s, | |
| | indazol-5-yl]- | 1H), 7.56(d, 1H), 7.68(m, | |
| | propionamide | 1H), 8.75(m, 1H), 10.00(s, | |
| | | 1H), 12.86 and 12.92(s, | |
| | | 1H), 13.45 and 13.49(s, 1H) | |
| 123 | Cyclopropanecarboxylic | 0.83(m, 4H), 1.21(m, 3H), | 429.6(ESI+) |
| | acid [3-(5-ethyl-7,7- | 1.33(s, 3H), 1.34(s, 3H), | |
| | dimethyl-6-oxo-1,5,6,7- | 1.84(m, 1H), 3.78(m, 2H), | |
| | tetrahydro-imidazo[4,5- | 7.02 and 7.34(s, 1H, two | |
| | f]indol-2-yl)-1H- | tautomeric forms), 7.43 and | |
| | indazol-5-yl]-amide | 7.69(s, 1H, two tautomeric | |
| | | forms), 7.56(m, 1H), | |
| | | 7.68(m, 1H), 8.77(d, 1H), | |
| | | 10.33(s, 1H), 12.86 and 12.92(br, | |
| | | 1H, two tautomeric forms), | |
| | | 13.45 and 13.49(br, 1H, two | |
| | | tautomeric forms) | |
| 124 | N-[3-(5-Ethyl-7,7- | 1.21(t, 3H), 1.33(s, 6H), | 465.6(ESI+) |
| | dimethyl-6-oxo-1,5,6,7- | 3.79(m, 2H), 7.19(br, 1H), | |
| | tetrahydro-imidazo[4,5- | 7.54-7.64(m, 5H), 7.83(m, | |
| | f]indol-2-yl)-1H- | 1H), 8.04(m, 1H), 8.06(s, | |
| | indazol-5-yl]-benzamide | 1H), 8.92(s, 1H), 10.43(br, | |
| | | 1H) | |
| 125 | Cyclohexanecarboxylic | 1.16-1.29(m, 5H), 1.27(m, | 491.4(ESI−) |
| | acid [3-(5-ethyl-7,7- | 2H), 1.34(m, 6H), 1.46(m, | |
| | dimethyl-6-oxo-1,5,6,7- | 2H), 1.68(m, 1H), | |
| | tetrahydro-imidazo[4,5- | 1.77-1.87(m, 3H), 2.34(m, 1H), | |
| | f]indol-2-yl)-1H- | 3.78(m, 2H), 7.02 and 7.36(s, | |
| | indazol-5-yl]-amide | 1H, tautomeric forms), 7.44 | |
| | | and 7.71(s, 1H, two | |
| | | tautomeric forms), 7.56(m, | |
| | | 1H), 7.65(m, 1H), 8.80(d, | |
| | | 1H), 9.94(s, 1H), 12.86 and | |
| | | 12.92(br, 1H, two | |
| | | tautomeric forms), 13.44 | |
| | | and 13.48(br, 1H, two | |
| | | tautomeric forms) | |
| 126 | 4-Methyl-piperazine-1- | 1.21(m, 3H), 1.33(s, 3H), | 487.3(ESI+) |
| | carboxylic acid [3-(5- | 1.34(s, 3H), 2.23(s, 3H), | |
| | ethyl-7,7-dimethyl-6- | 2.35(m, 4H), 3.49(m, 4H), | |
| | oxo-3,5,6,7-tetrahydro- | 3.79(m, 2H), 7.02 and | |
| | imidazo[4,5-f]indol-2- | 7.34(s, 1H, two tautomeric | |
| | yl)-1H-indazol-5-yl]- | forms), 7.43 and 7.69(s, 1H, | |
| | amide | two tautomeric forms), | |
| | | 7.51(m, 1H), 7.58(m, 1H), | |
| | | 8.48(m, 1H), 8.69(s, 1H), 12.85 | |
| | | and 12.91(br, 1H, two | |
| | | tautomeric forms), 13.38 | |
| | | and 13.43(br, 1H, 2 | |
| | | tautomeric forms) | |
| 127 | Piperidine-1-carboxylic | 1.21(t, 3H), 1.33(s, 6H), | 472.5(ESI+) |
| | acid [3-(5-ethyl-7,7- | 1.53(m, 4H), 1.61(m, 2H), | |
| | dimethyl-6-oxo-1,5,6,7- | 3.47(m, 4H), 3.78(q, 2H), | |
| | tetrahydro-imidazo[4,5- | 6.93-7.77(m, 2H), 7.50(d, | |
| | f]indol-2-yl)-1H- | 1H), 7.57(m, 1H), 8.47(s, | |
| | indazol-5-yl]-amide | 1H), 8.61(s, 1H), 12.89(bs, | |
| | | 1H), 13.40(s, 1H) | |
| 128 | Morpholine-4- | 1.21(t, 3H), 1.33(s, 6H), | 474.3(ESI+) |
| | carboxylic acid [3-(5- | 3.48(m, 4H), 3.65(m, 4H), | |
| | ethyl-7,7-dimethyl-6- | 3.78(m, 2H), 7.03 and | |
| | oxo-1,5,6,7-tetrahydro- | 7.32(bs, 1H), 7.44 and 7.67(bs, | |
| | imidazo[4,5-f]indol-2- | 1H), 7.52(d, 1H), 7.58(m, | |
| | yl)-1H-indazol-5-yl]- | 1H), 8.49(s, 1H), 8.72(s, | |
| | amide | 1H), 12.91(bs, 1H), | |
| | | 13.53(bs, 1H) | |
| 129 | Pyrrolidine-1-carboxylic | 1.21(t, 3H), 1.33(m, 6H), | 458.2(ESI+) |
| | acid [3-(5-ethyl-7,7- | 1.88(m, 4H), 3.42(m, 4H), | |
| | dimethyl-6-oxo-1,5,6,7- | 3.78(m, 2H), 7.02 and | |
| | tetrahydro-imidazo[4,5- | 7.34(s, 1H), 7.43 and 7.69(s, | |
| | f]indol-2-yl)-1H- | 1H), 7.50(m, 1H), 7.64(d, | |
| | indazol-5-yl]-amide | 1H), 8.30(s, 1H), 8.53(m, | |
| | | 1H), 12.83 and 12.89(s, | |
| | | 1H), 13.37 and 13.41(s, 1H) | |
| 130 | [3-(5-Ethyl-7,7- | 1.21(m, 3H), 1.33(m, 6H), | 495.2(ESI+) |
| | dimethyl-6-oxo-1,5,6,7- | 3.79(m, 2H), 5.21(s, 2H), | |
| | tetrahydro-imidazo[4,5- | 7.00-7.74(m, 2H), | |
| | f]indol-2-yl)-1H- | 7.32-7.51(m, 6H), 7.56(m, 1H), | |
| | indazol-5-yl]-carbamic | 8.72(m, 1H), 9.83(s, 1H), | |
| | acid benzyl ester | 12.85 and 12.91(s, 1H), | |
| | | 13.44 and 13.49(s, 1H) | |
| 131 | 4-Methyl-piperazine-1- | 1.21(m, 3H), 1.33(s, 6H), | 487.2(ESI+) |
| | carboxylic acid [3-(5- | 2.22(s, 3H), 2.35(m, 4H), | |
| | ethyl-7,7-dimethyl-6- | 3.49(m, 4H), 3.78(m, 2H), | |
| | oxo-1,5,6,7-tetrahydro- | 7.02 and 7.34(s, 1H), 7.43 | |
| | imidazo[4,5-f]indol-2- | and 7.69(s, 1H), 7.51(m, | |
| | yl)-1H-indazol-5-yl]- | 1H), 7.57(m, 1H), 8.48(m, | |
| | amide | 1H), 8.68(s, 1H), 12.84 and | |
| | | 12.90(s, 1H), 13.38 and | |
| | | 13.42(s, 1H) | |
| 132 | N-[3-(5-Ethyl-7,7- | 1.21(m, 3H), 1.33(s, 3H), | 501.1(ESI+) |
| | dimethyl-6-oxo-1,5,6,7- | 1.35(s, 3H), 3.79(m, 2H), | |
| | tetrahydro-imidazo[4,5- | 7.01 and 7.35(s, 1H, two | |
| | f]indol-2-yl)-1H- | tautomeric forms), 7.22(m, | |
| | indazol-5-yl]- | 1H), 7.42 and 7.74(s, 1H, | |
| | benzenesulfonamide | two tautomeric forms), | |
| | | 7.49-7.58(m, 4H), 7.73(m, | |
| | | 2H), 8.22(m, 1H), 10.19(s, | |
| | | 1H), 12.87 and 12.94(br, | |
| | | 1H, two tautomeric forms), | |
| | | 13.51 and 13.55(br, 1H, two | |
| | | tautomeric forms) | |
| 133 | N-[3-(5-Ethyl-7,7- | 1.21(m, 3H), 1.35(s, 6H), | 531.4(ESI+) |
| | dimethyl-6-oxo-1,5,6,7- | 3.74(s, 3H), 3.79(m, 2H), | |
| | tetrahydro-imidazo[4,5- | 7.02(d, 2H), 7.04 and | |
| | f]indol-2-yl)-1H- | 7.35(s, 1H, two tautomeric | |
| | indazol-5-yl]-4- | forms), 7.23(d, 1H), 7.43 | |
| | methoxy- | and 7.71(s, 1H, two | |
| | benzenesulfonamide | tautomeric forms), 7.51(d, | |
| | | 1H), 7.67(d, 2H), 8.20(br, | |
| | | 1H), 12.90(br, 1H), | |
| | | 13.52(br, 1H) | |
| 134 | N-[3-(5-Ethyl-7,7- | 1.22(m, 3H), 1.33(s, 3H), | 546.3(ESI+) |
| | dimethyl-6-oxo-1,5,6,7- | 1.35(s, 3H), 3.80(m, 2H), | |
| | tetrahydro-imidazo[4,5- | 7.02 and 7.36(s, 1H, two | |
| | f]indol-2-yl)-1H- | tautomeric forms), 7.26(m, | |
| | indazol-5-yl]-2-nitro- | 1H), 7.43 and 7.72(s, 1H, | |
| | benzenesulfonamide | two tautomeric forms), | |
| | | 7.58(dd, 1H), 7.77-7.84(m, 2H), | |
| | | 7.94-7.99(m, 2H), 8.28(dd, | |
| | | 1H), 10.60(s, 1H), 12.91 | |
| | | and 12.97(br, 1H, two | |
| | | tautomeric forms), 13.57 | |
| | | and 13.61(br, 1H, two | |
| | | tautomeric forms) | |
| 135 | N-[3-(5-Ethyl-7,7- | 1.22(m, 3H), 1.33(s, 3H), | 531.2(ESI+) |
| | dimethyl-6-oxo-1,5,6,7- | 1.35(s, 3H), 3.75-3.82(m, | |
| | tetrahydro-imidazo[4,5- | 5H), 7.02 and 7.34(s, 1H, | |
| | f]indol-2-yl)-1H- | two tautomeric forms), | |
| | indazol-5-yl]-3- | 7.12(m, 1H), 7.24(m, 1H), | |
| | methoxy- | 7.29-7.32(m, 2H), 7.40 and | |
| | benzenesulfonamide | 7.70(s, 1H, two tautomeric | |
| | | forms), 7.43(m, 1H), | |
| | | 7.54(dd, 1H), 8.27(dd, 1H), | |
| | | 10.19 and 10.21(br, 1H, two | |
| | | tautomeric forms), 12.89 | |
| | | and 12.95(br, 1H, two | |
| | | tautomeric forms), 13.52 | |
| | | and 13.57(br, 1H, two | |
| | | tautomeric forms) | |
| 136 | N-[3-(5-Ethyl-7,7- | 1.22(m, 3H), 1.33(s, 3H), | 585.3(ESI+) |
| | dimethyl-6-oxo-1,5,6,7- | 1.36(s, 3H), 3.80(m, 2H), | |
| | tetrahydro-imidazo[4,5- | 7.01 and 7.35(s, 1H, two | |
| | f]indol-2-yl)-1H- | tautomeric forms), 7.22(m, | |
| | indazol-5-yl]-2- | 1H), 7.43 and 7.74(s, 1H, | |
| | trifluoromethoxy- | two tautomeric forms), | |
| | benzenesulfonamide | 7.48-7.55(m, 3H), 7.71(m, | |
| | | 1H), 7.97(d, 1H), 8.24(d, | |
| | | 1H), 10.45(s, 1H), 12.88 | |
| | | and 12.94(br, 1H, two | |
| | | tautomeric forms), 13.51 | |
| | | and 13.56(br, 1H, two | |
| | | tautomeric forms) | |
| 137 | N-[3-(5-Ethyl-7,7- | 1.22(m, 3H), 1.33(s, 3H), | 516.9(ESI−) |
| | dimethyl-6-oxo-1,5,6,7- | 1.35(s, 3H), 3.80(m, 2H), | |
| | tetrahydro-imidazo[4,5- | 7.02 and 7.35(s, 1H, two | |
| | f]indol-2-yl)-1H- | tautomeric forms), 7.20(m, | |
| | indazol-5-yl]-4-fluoro- | 1H), 7.38(m, 2H), 7.43 and | |
| | benzenesulfonamide | 7.71(s, 1H, two tautomeric | |
| | | forms), 7.54(m, 1H), | |
| | | 7.79(m, 2H), 8.22(m, 1H), | |
| | | 10.20(s, 1H), 12.89 and | |
| | | 12.95(s, 1H, two tautomeric | |
| | | forms), 13.53 and 13.58(s, | |
| | | 1H, two tautomeric forms) | |
| 138 | 3-Chloro-N-[3-(5-ethyl- | 1.23(m, 3H), 1.34(m, 6H), | 535.0(ESI+) |
| | 7,7-dimethyl-6-oxo- | 3.79(m, 2H), 7.01 and | |
| | 1,5,6,7-tetrahydro- | 7.35(s, 1H), 7.19(m, 1H), 7.42 | |
| | imidazo[4,5-f]indol-2- | and 7.71(s, 1H), 7.53(m, | |
| | yl)-1H-indazol-5-yl]- | 2H), 7.65(m, 2H), 7.78(m, | |
| | benzenesulfonamide | 1H), 8.20(s, 1H), 10.32(bs, | |
| | | 1H), 12.88 and 12.93(s, | |
| | | 1H), 13.50 and 13.54(s, 1H) | |
| 139 | N-[3-(5-Ethyl-7,7- | 1.20(m, 3H), 1.34(m, 6H), | 515.1(ESI+) |
| | dimethyl-6-oxo-1,5,6,7- | 2.31(m, 3H), 3.79(m, 2H), | |
| | tetrahydro-imidazo[4,5- | 7.01 and 7.35(s, 1H), | |
| | f]indol-2-yl)-1H- | 7.22(m, 1H), 7.38(m, 2H), 7.43 | |
| | indazol-5-yl]-3-methyl- | and 7.71(s, 1H), 7.53(m, | |
| | benzenesulfonamide | 2H), 7.64(s, 1H), 8.24(m, | |
| | | 1H), 10.17(s, 1H), 12.88 | |
| | | and 12.94(s, 1H), 13.50 and | |
| | | 13.55(s, 1H) | |
| 140 | N-[3-(5-Ethyl-7,7- | 1.22(m, 3H), 1.34(m, 6H), | 579.4(ESI+) |
| | dimethyl-6-oxo-1,5,6,7- | 3.58(s, 3H), 3.80(m, 2H), | |
| | tetrahydro-imidazo[4,5- | 7.01 and 7.35(s, 1H), | |
| | f]indol-2-yl)-1H- | 7.17(m, 1H), 7.42 and 7.71(s, | |
| | indazol-5-yl]-2- | 1H), 7.52(d, 1H), 7.79(m, | |
| | methanesulfonyl- | 1H), 7.88(m, 1H), 7.98(d, | |
| | benzenesulfonamide | 1H), 8.27(m, 2H), 9.55(s, | |
| | | 1H), 12.89 and 12.95(s, | |
| | | 1H), 13.54 and 13.58(s, 1H) | |
| 141 | N-[3-(5-Ethyl-7,7- | 1.22(m, 3H), 1.34(m, 6H), | 537.4(ESI+) |
| | dimethyl-6-oxo-3,5,6,7- | 3.79(m, 2H), 7.01 and | |
| | tetrahydro-imidazo[4,5- | 7.35(s, 1H), 7.25(m, 1H), 7.43 | |
| | f]indol-2-yl)-1H- | and 7.71(s, 1H), | |
| | indazol-5-yl]-2,5- | 7.47-7.67(m, 4H), 8.25(m, 1H), | |
| | difluoro- | 10.69(s, 1H), 12.90 and | |
| | benzenesulfonamide | 12.95(s, 1H), 13.54 and | |
| | | 13.58(s, 1H) | |

In an analogous manner as described for example 121 the following examples 142-144 were prepared from 2-(5-Amino-1H-indazol-3-yl)-5-isopropyl-7,7-dimethyl-5,7-dihydro-1H-imidazo[4,5-f]indol-6-one (example 112) and the appropriate sulfonyl chlorides:

| Example | | ¹H-NMR(400MHz, | |
|---|---|---|---|
| No | Systematic Name | DMSO): δ(ppm)= | MS: M= |
| 142 | 4-Fluoro-N-[3-(5- | 1.31(s, 3H), 1.33(s, 3H), | 532.9(API+) |
| | isopropyl-7,7-dimethyl- | 1.47(d, 3H), 1.50(d, 3H), | |
| | 6-oxo-1,5,6,7- | 4.60(m, 1H), 7.12 and | |
| | tetrahydro-imidazo[4,5- | 7.37(s, 1H, two tautomeric | |
| | f]indol-2-yl)-1H- | forms), 7.20(t, 1H), | |
| | indazol-5-yl]- | 7.35-7.41(m, 2H), 7.42 and | |
| | benzenesulfonamide | 7.70(s, 1H, two tautomeric | |
| | | forms), 7.53(m, 1H), | |
| | | 7.79(dd, 2H), 8.20(s, 1H), 12.80 | |
| | | and 12.93(br, 1H, two | |
| | | tautomeric forms), 13.53 | |
| | | and 13.58(br, 1H, two | |
| | | tautomeric forms) | |
| 143 | N-[3-(5-Isopropyl-7,7- | 1.33(m, 6H), 1.49(m, 6H), | 593.2(ESI+) |
| | dimethyl-6-oxo-1,5,6,7- | 3.58(m, 3H), | |
| | tetrahydro-imidazo[4,5- | 4.51-4.69(m, 1H), 7.12 and 7.51(s, | |
| | f]indol-2-yl)-1H- | 1H), 7.16(m, 1H), 7.41(d, | |
| | indazol-5-yl]-2- | 1H), 7.53 and 7.70(s, 1H), | |
| | methanesulfonyl- | 7.79(m, 1H), 7.87(t, 1H), | |
| | benzenesulfonamide | 7.98(m, 1H), | |
| | | 8.21-8.31(m, 2H), 9.54(s, 1H), 12.79 | |
| | | and 12.92(s, 1H), 13.53 and | |
| | | 13.57(s, 1H) | |
| 144 | N-[3-(5-Isopropyl-7,7- | 1.32(s, 3H), 1.34(s, 3H), | 558.2(ESI−) |
| | dimethyl-6-oxo-1,5,6,7- | 1.47(d, 3H), 1.50(d, 3H), | |
| | tetrahydro-imidazo[4,5- | 4.60(m, 1H), 7.13 and | |
| | f]indol-2-yl)-1H- | 7.42(s, 1H, two tautomeric | |
| | indazol-5-yl]-2-nitro- | forms), 7.26(m, 1H), 7.42 | |
| | benzenesulfonamide | and 7.71(s, 1H, two | |
| | | tautomeric forms), 7.57(d, | |
| | | 1H), 7.80(m, 2H), 7.97(m, | |
| | | 2H), 8.28(m, 1H), | |
| | | 10.60(br, 1H), 12.81 and | |
| | | 12.93(br, 1H, two tautomeric | |
| | | forms), 13.55 and 13.59(br, | |
| | | 1H, two tautomeric forms) | |

### Example 145

### N-[3-(5-Ethyl-7,7-dimethyl-6-oxo-3,5,6,7-tetrahydro-imidazo[4,5-f]indol-2-yl)-1H-indazol-5-yl]-acetamide

To a solution of 2-(5-amino-1H-indazol-3-yl)-5-ethyl-7,7-dimethyl-5,7-dihydro-3H-imidazo[4,5-f]indol-6-one (example 111, 220 mg, 0.610 mmol) in pyridine (3 ml) was added acetic anhydride (623 mg, 576 μl, 6.10 mmol). After 12 h at room temperature the pyridine was evaporated and the residue was treated with CHCl₃ (5 ml), MeOH (10 ml) and KOH (1M, 3 ml). After 6 h at room temperature water was added and the aqueous phase extracted three times with ethyl acetate. The combined organic phases were washed with HCl solution (1M) and brine, were dried over MgSO₄ and the solvent was evaporated to yield 225 mg N-[3-(5-ethyl-7,7-dimethyl-6-oxo-3,5,6,7-tetrahydro-imidazo[4,5-f]indol-2-yl)-1H-indazol-5-yl]-acetamide (0.559 mmol, 92%).

MS: M=403.2 (ESI+)

¹H-NMR (400 MHz, DMSO): δ (ppm)=1.21 (m, 3H), 1.33 (s, 3H), 1.34 (s, 3H), 2.09 (s, 3H), 3.78 (m, 2H), 7.03 and 7.34 (s, 1H, two tautomeric forms), 7.44 and 7.69 (s, 1H, two tautomeric forms), 7.57 (m, 1H), 7.69 (m, 1H), 8.70 (m, 1H), 10.08 (br, 1H), 12.87 and 12.93 (s, 1H, two tautomeric forms), 13.46 and 13.50 (s, 1H, two tautomeric forms)

### Example 146

### 4-Acetyl-piperazine-1-carboxylic acid [3-(5-ethyl-7,7-dimethyl-6-oxo-1,5,6,7-tetrahydro-imidazo[4,5-f]indol-2-yl)-1H-indazol-5-yl]-amide

To a solution 2-(5-amino-1H-indazol-3-yl)-5-ethyl-7,7-dimethyl-5,7-dihydro-3H-imidazo[4,5-f]indol-6-one (example 111, 200 mg, 0.555 mmol) in absolute THF (15 ml) under a nitrogen atmosphere was added 1,1′-carbonyl-diimidazole (432 mg, 2.64 mmol). After heating under reflux for 12 h a solution of 1-acetylpiperazine (356 mg, 2.77 mmol) in THF (3 ml) was added and the reaction mixture was again heated under reflux for 12 h. The solvent was evaporated and methanol (5 ml) and KOH (1M solution, 1 ml) were added. After 4 h at room temperature water was added and the aqueous phase extracted three times with ethyl acetate. The combined organic phases were washed with HCl solution (1M) and brine, dried over MgSO₄ and the solvent was evaporated. The residue was purified by HPLC chromatography to yield 75 mg 4-acetyl-piperazine-1-carboxylic acid [3-(5-ethyl-7,7-dimethyl-6-oxo-1,5,6,7-tetrahydro-imidazo[4,5-f]indol-2-yl)-1H-indazol-5-yl]-amide (0.146 mmol, 26%).

MS: M=515.5 (ESI+)

¹H-NMR (400 MHz, DMSO): δ (ppm)=1.21 (m, 3H), 1.33 (m, 6H), 2.06 (s, 3H), 3.45-3.57 (m, 8H), 3.79 (m, 2H), 7.02 and 7.33 (s, 1H), 7.43 and 7.68 (s, 1H), 7.52 (m, 1H), 7.59 (m, 1H), 8.49 (m, 1H), 8.78 (s, 1H), 12.85 and 12.91 (s, 1H), 12.39 and 13.44 (s, 1H)

According to the described examples 1-146 and the schemes 1-4 the following examples 147-148 can be prepared from the appropriate starting materials:

| Example | |
|---|---|
| No | Systematic Name |
| 147 | 2-(5-Benzylamino-1H-indazol-3-yl)-5-ethyl-7,7-dimethyl-5,7- |
| | dihydro-1H-imidazo[4,5-f]indol-6-one |
| 148 | 2-(5-Benzyloxy-1H-indazol-3-yl)-5-ethyl-7,7-dimethyl-5,7- |
| | dihydro-1H-imidazo[4,5-f]indol-6-one |

Unless stated to the contrary, all compounds in the examples were prepared and characterized as described. All ranges recited herein encompass all combinations and subcombinations included within that range limit. All patents and publications cited herein are hereby incorporated by reference in their entirety for any purpose.

1. A compound of formula I and all pharmaceutically acceptable salts or esters thereof wherein formula I is: 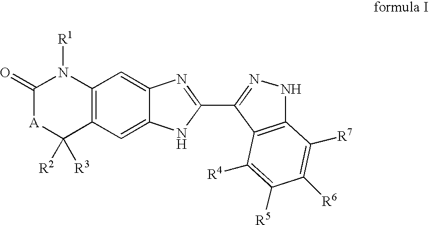
wherein:
(a) R¹ is selected from the group consisting of:
(1) hydrogen;
(2) alkyl, which is optionally substituted one or more times with halogen, hydroxy, alkoxy, amino, alkylamino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH—, or phenyl-S(O)₂—NH—;
(3) alkenyl, which is optionally substituted one or more times with halogen, hydroxy, alkoxy, amino, alkylamino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH—, or phenyl-S(O)₂—NH—;
(4) alkynyl, which is optionally substituted one or more times with halogen, hydroxy, alkoxy, amino, alkylamino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH—, or phenyl-S(O)₂—NH—;
(5) arylalkyl, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄)alkyl, halogenated (C₁-C₄)alkoxy or alkylsulfonyl;
(6) heteroarylalkyl,
wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl or halogen;
(7) heterocyclyl-C(O)—(CH₂)ₙ—;
(8) R⁸—NH—C(O)—(CH₂)ₙ—; and
(9) R⁹—C(O)—NH—(CH₂)ₙ—;
(b) R⁸ is selected from the group consisting of:
(1) hydroxyl;
(2) alkoxy;
(3) benzyloxy;
(4) alkyl, which is optionally substituted one to three times with halogen, hydroxy, alkoxy, amino, alkylamino or dialkylamino;
(5) phenyl-(CH₂)ₘ—, wherein the phenyl is optionally substituted one to three times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
(6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
(1) cycloalkyl, wherein said alkyl is optionally substituted one to three times with halogen, hydroxy, alkoxy, amino, alkylamino or dialkylamino;
(2) heterocyclyl;
(3) benzylamino;
(4) alkyl, wherein said alkyl is optionally substituted one to three times with halogen, hydroxy, alkoxy, amino, alkylamino or dialkylamino;
(5) phenyl-(CH₂)ₘ—, wherein the phenyl is optionally substituted one to three times with halogen, cyano, nitro, amino, hydroxy, (C₁-C₄)alkyl, (C₁-C₄)alkoxy, halogenated (C₁-C₄)alkyl or halogenated (C₁-C₄)alkoxy; and
(6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) n is 1, 2 or 3;
(e) m is 0 or 1;
(f) R² and R³ form together with the carbon atom to which they are attached a cycloalkyl ring; or alternatively, R² is hydrogen or alkyl and R³ is hydrogen or alkyl;
(g) R⁴ is hydrogen or halogen;
(h) R⁷ is hydrogen or halogen;
(i) R⁵ is selected from the group consisting of:
(1) hydrogen;
(2) halogen;
(3) cyano;
(4) nitro;
(5) amino;
(6) hydroxyl;
(7) sulfonic acid;
(8) carboxylic acid;
(9) CH₃O—C(O)—;
(10) H₂N—C(O)—;
(11) CH₃O—N(CH₃)—C(O)—;
(12) cycloalkyl-X—, wherein the alkyl is optionally substituted one or more times with halogen;
(13) heterocyclyl-X—;
(14) alkyl, which is optionally substituted one or more times with halogen;
(15) alkyl-X—, wherein the alkyl is optionally substituted one or more times with halogen;
(16) aryl-X—, wherein the aryl is optionally substituted one or more times with halogen, cyano, nitro, amino, hydroxy, (C₁-C₄)alkyl, (C₁-C₄)alkoxy, halogenated (C₁-C₄) alkyl, halogenated (C₁-C₄)alkoxy, or alkylsulfonyl;
(17) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
(18) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl; and
(19) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(j) R⁶ is selected from group consisting of:
(1) hydrogen;
(2) halogen;
(3) cyano;
(4) nitro;
(5) amino;
(6) hydroxy;
(7) sulfonic acid;
(8) carboxylic acid;
(9) CH₃O—C(O)—;
(10) H₂N—C(O)—;.
(11) CH₃O—N(CH₃)—C(O)—;
(12) cycloalkyl-X—, wherein the alkyl is optionally substituted one or more times with halogen;
(13) heterocyclyl-X—;
(14) alkyl, which is optionally substituted one or more times with halogen;
(15) alkyl-X—, wherein the alkyl is optionally substituted one or more times with halogen;
(16) aryl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, halogenated (C₁-C₄)alkoxy, or alkylsulfonyl;
(17) arylalkyl-X—, wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
(18) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl; and
(19) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(k) X is —NH—, —N(alkyl)-, —O—, —S(O)₂NH—, —NHS(O)₂—, —NHC(O)—, —N(alkyl)C(O)—, —C(O)—, —OC(O)NH—, —C(O)NH— or —C(O)N(alkyl)—; and
(l) A is a single bond or —CH₂—.

2. The compounds of claim 1, wherein:
(a) R¹ is selected from the group consisting of:
(1) hydrogen;
(2) alkly, which is optionally substituted one or more times with halogen, hydroxy, alkoxy, amino, alkylamino, dialkylamino, cycloalkyl, heterocyclyl, or dialkylphosphinoyl;
(3) alkenyl, which is optionally substituted one or more times with halogen, hydroxy, alkoxy, amino, alkylamino, dialkylamino, cycloalkyl, heterocyclyl, or dialkylphosphinoyl;
(4) alkynyl, which is optionally substituted one or more times with halogen, hydroxy, alkoxy, amino, alkylamino, dialkylamino, cycloalkyl, heterocyclyl, or dialkylphosphinoyl;
(5) arylalkyl, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
(6) heteroarylalkyl,
wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl or halogen;
(b) R⁵ is selected from the group consisting of:
(1) hydrogen;
(2) halogen;
(3) cyano;
(4) nitro;
(5) amino;
(6) hydroxyl;
(7) sulfonic acid;
(8) carboxylic acid;
(9) alkyl, which is optionally substituted one or more times with halogen;
(10) alkyl-X—, wherein the alkyl is optionally substituted one or more times with halogen;
(11) aryl-X—, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄) alkyl, or halogenated (C₁-C₄)alkoxy;
(12) arylalkyl-X—, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy; and
(13) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms, and wherein the heteroaryl is optionally substituted one or more times with alkyl; and
(c) R⁶ is selected from group consisting of:
(1) hydrogen;
(2) halogen;
(3) cyano;
(4) nitro;
(5) amino;
(6) hydroxy;
(7) sulfonic acid;
(8) carboxylic acid;
(9) alkyl, which is optionally substituted one or more times with halogen;
(10) alkyl-X—, wherein the alkyl is optionally substituted one or more times with halogen;
(11) aryl-X—, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄)alkyl, or halogenated (C₁-C₄)alkoxy;
(12) arylalkyl-X—, wherein the aryl is a mono- or bicyclic aromatic ring with 6 to 10 ring carbon atoms and wherein the aryl is optionally substituted one or more times with halogen, cyano, nitro, amino, hydroxy, (C₁-C₄)alkyl, (C₁-C₄)alkoxy, halogenated (C₁-C₄)alkyl or halogenated (C₁-C₄)alkoxy; and
(13) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms and wherein the heteroaryl is optionally substituted one or more times with alkyl;
(d) X is —NH—, —N(alkyl)-, —O—, —S(O)₂NH—, —NHS(O)₂—, —NHC(O)—,—N(alkyl)C(O)—, —C(O)NH— or —C(O)N(alkyl)-.

3. The compounds of claim 1, wherein:
(a) R¹ is selected from the group consisting of:
(1) hydrogen;
(2) alkyl, which is optionally substituted one or more times with hydroxy, alkoxy, amino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH— or phenyl-S(O)₂—NH—;
(3) alkenyl,
(4) arylalkyl, wherein the aryl is a mono- or bicyclic aromatic ring with 6 to 10 ring carbon atoms and wherein the aryl is optionally substituted one or more times with alkylsulfonyl,
(5) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(6) heterocyclyl-C(O)—(CH₂)ₙ—;
(7) R⁸—NH—C(O)—(CH₂)ₙ—; and
(8) R⁹—C(O)—NH—(CH₂)ₙ—;
(b) R⁸ is selected from the group consisting of:
(1) hydroxy,
(2) alkoxy,
(3) benzyloxy,
(4) alkyl, which is optionally substituted one to three times with hydroxy or dialkylamino;
(5) phenyl-(CH₂)ₘ—, wherein the phenyl is optionally substituted one to three times with halogen or (C₁-C₄)alkoxy; and
(6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
(1) cycloalkyl;
(2) heterocyclyl;
(3) benzylamino;
(4) alkyl;
(5) phenyl-(CH₂)ₘ—; and
(6)heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) n is 1, 2 or 3;
(e) m is 0 or 1;
(f) R⁴ and R⁷ each represent hydrogen;
(g) R⁵ is selected from the group consisting of:
(1) hydrogen;
(2) halogen;
(3) cyano;
(4) nitro;
(5) amino;
(6) carboxylic acid;
(7) CH₃O—C(O)—;
(8) H₂N—C(O)—;
(9) CH₃O—N(CH₃)—C(O)—;
(10) cycloalkyl-X—;
(11) heterocyclyl-X—;
(12) alkyl-X—, wherein the alkyl group is optionally substituted one or more times with halogen;
(13) aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
(14) arylalkyl-X—, wherein the aryl is a mono- or bicyclic aromatic ring with 6 to 10 ring carbon atoms and wherein the aryl is optionally substituted one or more times with halogen, (C₁-C₄)alkyl, (C₁-C₄)alkoxy or halogenated (C₁-C₄)alkoxy;
(15) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms; and
(16) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(h) R⁶ is selected from the group consisting of:
(1) hydrogen;
(2) halogen;
(3) carboxylic acid;
(4) H₂N—C(O)—;
(5) alkyl-X—;
(6) aryl-X—, wherein the aryl is a mono- or bicyclic aromatic ring with 6 to 10 ring carbon atoms; and
(7) arylalkyl-X—, wherein the aryl is a mono- or bicyclic aromatic ring with 6 to 10 ring carbon atoms; and
(i) X is selected from the group consisting of —NH—, —O—, —S(O)₂NH—, —NHC(O)—, —C(O)—, —OC(O)NH— or —C(O)NH—.

4. The compounds of claim 1, wherein A is a single bond.

5. The compounds of claim 4, wherein:
(a) R¹ is selected from the group consisting of:
(1) hydrogen;
(2) alkyl; wherein said alkyl is optionally substituted one or more times with hydroxy, alkoxy, amino, dialkylamino, cycloalkyl, heterocyclyl, dialkylphosphinoyl, alkoxyalkoxy, alkyl-O—C(O)—, cyano, alkylsulfanyl, alkylsulfinyl, alkylsulfonyl, (alkyl)₃Si—O—, H₂N—C(S)—, HO—C(O)—, H₂N—C(O)—, alkyl-S(O)₂—NH— or phenyl-S(O)₂—NH—;
(3) alkenyl;
(4) arylalkyl, wherein the aryl is a mono- or bicyclic aromatic ring with 6 to 10 ring carbon atoms and wherein the aryl is optionally substituted one or more times with alkylsulfonyl;
(5) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(6) heterocyclyl-C(O)—(CH₂)ₙ—;
(7) R⁸—NH—C(O)—(CH₂)ₙ—; and
(8) R⁹—C(O)—NH—(CH₂)ₙ—;
(b) R⁸ is selected from the group consisting of:
(1) hydroxyl;
(2) alkoxy;
(3) benzyloxy;
(4) alkyl, wherein said alkyl is optionally substituted one to three times with hydroxy or dialkylamino;
(5) phenyl-(CH₂)ₘ—, wherein the phenyl is optionally substituted one to three times with halogen or (C₁-C₄)alkoxy; and
(6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(c) R⁹ is selected from the group consisting of:
(1) cycloalkyl;
(2) heterocyclyl;
(3) benzylamino;
(4) alkyl;
(5) phenyl-(CH₂)ₘ—; and
(6) heteroaryl-(CH₂)ₘ—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) n is 1, 2 or 3; and
(e) m is 0 or 1.

6. The compounds of claim 4, wherein R¹ is hydrogen; and R⁴, R⁵, R⁶ and R⁷ each represent hydrogen.

7. A compound of claim 1 selected from the group consisting of:
2-(1H-Indazol-3-yl)-7,7-dimethyl-5,7-dihydro-3H-imidazo[4,5-f]indol-6-one;
2-(1H-Indazol-3-yl)-spiro[7,7-cyclopentan-5,7-dihydro-3H-imidazo[4,5-f]indol-6]-one;
2-(1H-Indazol-3-yl)-7-methyl-5,7-dihydro-3H-imidazo[4,5-f]indol-6-one; and
7-Ethyl-2-(1H-indazol-3-yl)-5,7-dihydro-3H-imidazo[4,5-f]indol-6-one.

8. The compounds of claim 4, wherein R¹ is alkyl or alkenyl; and R⁴, R⁵, R⁶ and R⁷ each represent hydrogen.

9. A compound of claim 1 selected from the group consisting of:
5-Allyl-2-(1H-indazol-3-yl)-7,7-dimethyl-5,7-dihydro-3H-imidazo[4,5-f]indol-6-one;
5-Ethyl-2-(1H-indazol-3-yl)-7,7-dimethyl-5,7-dihydro-3H-imidazo[4,5-f]indol-6-one;
2-(1H-Indazol-3-yl)-5,7,7-trimethyl-5,7-dihydro-3H-imidazo[4,5-f]indol-6-one;
2-(1H-Indazol-3-yl)-7,7-dimethyl-5-propyl-5,7-dihydro-3H-imidazo[4,5-f]indol-6-one;
2-(1H-Indazol-3-yl)-5-isopropyl-7,7-dimethyl-5,7-dihydro-3H-imidazo[4,5-f]indol-6-one;
5,7,7-Triethyl-2-(1H-indazol-3-yl)-5,7-dihydro-3H-imidazo[4,5-f]indol-6-one; and
5-But-3-enyl-2-(1H-indazol-3-yl)-7,7-dimethyl-5,7-dihydro-3H-imidazo[4,5-f]indol-6-one.

10. The compounds of claim 4, wherein:
(a) R¹ is alkyl, wherein said alkyl is substituted one to three times with hydroxy, alkoxy, amino, dialkylamino, dialkylphosphinoyl, alkoxyalkoxy, cyano, cycloalkyl, heterocyclyl, alkylsulfanyl, alkylsulfinyl or alkylsulfonyl; and
(b) R⁴, R⁵, R⁶ and R⁷ each represent hydrogen.

11. A compound of claim 1 selected from the group consisting of:
5-Cyclopropylmethyl-2-(1H-indazol-3-yl)-7,7-dimethyl-5,7-dihydro-3H-imidazo[4,5-f]indol-6-one;
2-(1H-Indazol-3-yl)-7,7-dimethyl-5-(3-morpholin-4-yl-propyl)-5,7-dihydro-3H-imidazo[4,5-f]indol-6-one;
2-(1H-Indazol-3-yl)-5-[2-(2-methoxy-ethoxy)-ethyl]-7,7-dimethyl-5,7-dihydro-3H-imidazo[4,5-f]indol-6-one;
2-(1H-Indazol-3-yl)-5-(2-methoxy-ethyl)-7,7-dimethyl-5,7-dihydro-3H-imidazo[4,5-f]indol-6-one;
2-(1H-Indazol-3-yl)-7,7-dimethyl-5-(3-piperidin-1-yl-propyl)-5,7-dihydro-3H-imidazo[4,5-f]indol-6-one;
5-(2-Diisopropylamino-ethyl)-2-(1H-indazol-3-yl)-7,7-dimethyl-5,7-dihydro-3H-imidazo[4,5-f]indol-6-one;
5-(3-Dimethylamino-propyl)-2-(1H-indazol-3-yl)-7,7-dimethyl-5,7-dihydro-3H-imidazo[4,5-f]indol-6-one;
5-(2-Diethylamino-ethyl)-2-(1H-indazol-3-yl)-7,7-dimethyl-5,7-dihydro-3H-imidazo[4,5-f]indol-6-one; and
[2-(1H-Indazol-3-yl)-7,7-dimethyl-6-oxo-6,7-dihydro-3H-imidazo[4,5-f]indol-5-yl]-acetonitrile;

12. A compound of claim 1 selected from the group consisting of:
2-(1H-Indazol-3-yl)-7,7-dimethyl-5-(2-methylsulfanyl-ethyl)-5,7-dihydro-3H-imidazo[4,5-f]indol-6-one;
5-(2-Hydroxy-3-morpholin-4-yl-propyl)-2-(1H-indazol-3-yl)-7,7-dimethyl-5,7-dihydro-3H-imidazo[4,5-f]indol-6-one;
5-(Dimethyl-phosphinoylmethyl)-2-(1H-indazol-3-yl)-7,7-dimethyl-5,7-dihydro-3H-imidazo[4,5-f]indol-6-one;
5-(2-Hydroxy-ethyl)-2-(1H-indazol-3-yl)-7,7-dimethyl-5,7-dihydro-3H-imidazo[4,5-f]indol-6-one;
5-(2,3-Dihydroxy-propyl)-2-(1H-indazol-3-yl)-7,7-dimethyl-5,7-dihydro-3H-imidazo[4,5-f]indol-6-one;
5-(2-Amino-ethyl)-2-(1H-indazol-3-yl)-7,7-dimethyl-5,7-dihydro-3H-imidazo[4,5-f]indol-6-one;
2-(1H-Indazol-3-yl)-5-(2-methanesulfinyl-ethyl)-7,7-dimethyl-5,7-dihydro-3H-imidazo[4,5-f]indol-6-one; and
2-(1H-Indazol-3-yl)-5-(2-methanesulfonyl-ethyl)-7,7-dimethyl-5,7-dihydro-3H-imidazo[4,5-f]indol-6-one.

13. The compounds of claim 4, wherein:
(a) R¹ is alkyl, which is substituted one or more times with alkyl-O—C(O)—, (alkyl)₃Si—O—, H₂N—C(S)—, HO—C(O)—, H₂N—C(O)—, alkyl-S(O)₂—NH— or phenyl-S(O)₂—NH—; and
(b) R⁴, R⁵, R⁶ and R⁷ each represent hydrogen.

14. A compound of claim 1 selected from the group consisting of:
[2-(1H-Indazol-3-yl)-7,7-dimethyl-6-oxo-6,7-dihydro-3H-imidazo[4,5-f]indol-5-yl]-acetic acid ethyl ester;
5-[2-(tert-Butyl-dimethyl-silanyloxy)-ethyl]-2-(1H-indazol-3-yl)-7,7-dimethyl-5,7-dihydro-3H-imidazo[4,5-f]indol-6-one;
2-[2-(1H-Indazol-3-yl)-7,7-dimethyl-6-oxo-6,7-dihydro-3H-imidazo[4,5-f]indol-5-yl]-thioacetamide;
[2-(1H-Indazol-3-yl)-7,7-dimethyl-6-oxo-6,7-dihydro-3H-imidazo[4,5-f]indol-5-yl]-acetic acid;
2-[2-(1H-Indazol-3-yl)-7,7-dimethyl-6-oxo-6,7-dihydro-3H-imidazo[4,5-f]indol-5-yl]-acetamide;
N-{2-[2-(1H-Indazol-3-yl)-7,7-dimethyl-6-oxo-6,7-dihydro-3H-imidazo[4,5-f]indol-5-yl]-ethyl}-benzenesulfonamide; compound with acetic acid; and
N-{2-[2-(1H-Indazol-3-yl)-7,7-dimethyl-6-oxo-6,7-dihydro-3H-imidazo[4,5-f]indol-5-yl]-ethyl}-methanesulfonamide.

15. The compounds of claim 4, wherein:
(a) R¹ is selected from the group consisting of:
(1) arylalkyl, wherein the aryl is a mono- or bicyclic aromatic ring with 6 to 10 ring carbon atoms and wherein the aryl is optionally substituted one or more times with alkylsulfonyl; and
(2) heteroarylalkyl, wherein the heteroaryl is a mono- or bicyclic aromatic ring with 5 to 10 ring atoms, which contains up to 4 heteroatoms selected independently from the group consisting of N, O and S with the remaining ring atoms being carbon atoms; and
(b) R⁴, R⁵, R⁶ and R⁷ each represent hydrogen.

16. A compound of claim 1 selected from the group consisting of:
2-(1H-Indazol-3-yl)-7,7-dimethyl-5-pyridin-3-ylmethyl-5,7-dihydro-3H-imidazo[4,5-f]indol-6-one;
5-Benzyl-2-(1H-indazol-3-yl)-7,7-dimethyl-5,7-dihydro-3H-imidazo[4,5-f]indol-6-one;
2-(1H-Indazol-3-yl)-5-(4-methanesulfonyl-benzyl)-7,7-dimethyl-5,7-dihydro-3H-imidazo[4,5-f]indol-6-one;
2-(1H-Indazol-3-yl)-7,7-dimethyl-5-thiazol-2-ylmethyl-5,7-dihydro-3H-imidazo[4,5-f]indol-6-one; and
2-(1H-Indazol-3-yl)-7,7-dimethyl-5-(1H-tetrazol-5-ylmethyl)-5,7-dihydro-3H-imidazo[4,5-f]indol-6-one.

17. The compounds of claim 4, wherein:
R¹ is heterocyclyl-C(O)—(CH₂)ₙ—; and
R⁴, R⁵, R⁶ and R⁷ each represent hydrogen.

18. A compound of claim 1 selected from the group consisting of,
2-(1H-Indazol-3-yl)-7,7-dimethyl-5-(2-morpholin-4-yl-2-oxo-ethyl)-5,7-dihydro-3H-imidazo[4,5-f]indol-6-one;
2-(1H-Indazol-3-yl)-7,7-dimethyl-5-[2-(4-methyl-piperazin-1-yl)-2-oxo-ethyl]-5,7-dihydro-3H-imidazo[4,5-f]indol-6-one; and
2-(1H-Indazol-3-yl)-7,7-dimethyl-5-(2-oxo-2-piperidin-1-yl-ethyl)-5,7-dihydro-3H-imidazo[4,5-f]indol-6-one.

19. The compounds of claim 4, wherein:
(a) R¹ is R⁸—NH—C(O)—(CH₂)ₙ—; and
(b) R⁴, R⁵, R⁶ and R⁷ each represent hydrogen.

20. A compound of claim 1 selected from the group consisting of:
N-(2-Dimethylamino-ethyl)-2-[2-(1H-indazol-3-yl)-7,7-dimethyl-6-oxo-6,7-dihydro-3H-imidazo[4,5-f]indol-5-yl]-acetamide;
N-Benzyl-2-[2-(1H-indazol-3-yl)-7,7-dimethyl-6-oxo-6,7-dihydro-3H-imidazo[4,5-f]indol-5-yl]-acetamide;
2-[2-(1H-Indazol-3-yl)-7,7-dimethyl-6-oxo-6,7-dihydro-3H-imidazo[4,5-f]indol-5-yl]-N-pyridin-3-ylmethyl-acetamide;
2-[2-(1H-Indazol-3-yl)-7,7-dimethyl-6-oxo-6,7-dihydro-3H-imidazo[4,5-f]indol-5-yl]-N-phenyl-acetamide;
N-(4-Fluoro-phenyl)-2-[2-(1H-indazol-3-yl)-7,7-dimethyl-6-oxo-6,7-dihydro-3H-imidazo[4,5-f]indol-5-yl]-acetamide;
N-(4-Fluoro-benzyl)-2-[2-(1H-indazol-3-yl)-7,7-dimethyl-6-oxo-6,7-dihydro-3H-imidazo[4,5-f]indol-5-yl]-acetamide;
N-(3,5-Dimethoxy-benzyl)-2-[2-(1H-indazol-3-yl)-7,7-dimethyl-6-oxo-6,7-dihydro-3H-imidazo[4,5-f]indol-5-yl]-acetamide;
N-(2,3-Dihydroxy-propyl)-2-[2-(1H-indazol-3-yl)-7,7-dimethyl-6-oxo-6,7-dihydro-3H-imidazo[4,5-f]indol-5-yl]-acetamide;
N-Hydroxy-2-[2-(1H-indazol-3-yl)-7,7-dimethyl-6-oxo-6,7-dihydro-3H-imidazo[4,5-f]indol-5-yl]-acetamide;
N-Benzyloxy-2-[2-(1H-indazol-3-yl)-7,7-dimethyl-6-oxo-6,7-dihydro-3H-imidazo[4,5-f]indol-5-yl]-acetamide; and
2-[2-(1H-Indazol-3-yl)-7,7-dimethyl-6-oxo-6,7-dihydro-3H-imidazo[4,5-f]indol-5-yl]-N-methoxy-acetamide.

21. The compounds of claim 4, wherein:
(a) R¹ is R⁹—C(O)—NH—(CH₂)ₙ—; and
(b) R⁴, R⁵, R⁶ and R⁷ each represent hydrogen.

22. A compound of claim 1 selected from the group consisting of:
N-{2-[2-(1H-Indazol-3-yl)-7,7-dimethyl-6-oxo-6,7-dihydro-3H-imidazo[4,5-f]indol-5-yl]-ethyl}-benzamide;
N-{2-[2-(1H-Indazol-3-yl)-7,7-dimethyl-6-oxo-6,7-dihydro-3H-imidazo[4,5-f]indol-5-yl]-ethyl}-2-phenyl-acetamide;
N-{2-[2-(1H-Indazol-3-yl)-7,7-dimethyl-6-oxo-6,7-dihydro-3H-imidazo[4,5-f]indol-5-yl]-ethyl}-nicotinamide;
Cyclopropanecarboxylic acid {2-[2-(1H-indazol-3-yl)-7,7-dimethyl-6-oxo-6,7-dihydro-3H-imidazo[4,5-f]indol-5-yl]-ethyl}-amide;
Morpholine-4-carboxylic acid {2-[2-(1H-indazol-3-yl)-7,7-dimethyl-6-oxo-6,7-dihydro-3H-imidazo[4,5-f]indol-5-yl]-ethyl}-amide;
Pyrrolidine-1-carboxylic acid {2-[2-(1H-indazol-3-yl)-7,7-dimethyl-6-oxo-6,7-dihydro-3H-imidazo[4,5-f]indol-5-yl]-ethyl}-amide;
4-Methyl-piperazine-1-carboxylic acid {2-[2-(1H-indazol-3-yl)-7,7-dimethyl-6-oxo-6,7-dihydro-3H-imidazo[4,5-f]indol-5-yl]-ethyl}-amide;
N-{2-[2-(1H-Indazol-3-yl)-7,7-dimethyl-6-oxo-6,7-dihydro-3H-imidazo[4,5-f]indol-5-yl]-ethyl}-acetamide; and
1-Benzyl-3-{2-[2-(1H-indazol-3-yl)-7,7-dimethyl-6-oxo-6,7-dihydro-3H-imidazo[4,5-f]indol-5-yl]-ethyl}-urea.

23. The compounds of claim 4, wherein
(a) R¹ is hydrogen or alkyl;
(b) R⁴ and R⁷ each represent hydrogen; and
(c) R⁶ is hydrogen.

24. The compounds of claim 4, wherein:
(a) R¹ is alkyl;
(b) R⁴ and R⁷ each represent hydrogen;
(c) R⁵ is halogen, cyano, nitro, amino, carboxylic acid, CH₃O—C(O)—, H₂N—C(O)— or CH₃O—N(CH₃)—C(O)—; and
(d) R⁶ is hydrogen.

25. A compound of claim 1 selected from the group consisting of:
5-Ethyl-2-(5-fluoro-1H-indazol-3-yl)-7,7-dimethyl-5,7-dihydro-3H-imidazo[4,5-f]indol-6-one;
2-(5-Chloro-1H-indazol-3-yl)-5-ethyl-7,7-dimethyl-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-1H-indazole-5-carboxylic acid;
5-Ethyl-7,7-dimethyl-2-(5-nitro-1H-indazol-3-yl)-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-1H-indazole-5-carbonitrile;
2-(5-Bromo-1H-indazol-3-yl)-5-ethyl-7,7-dimethyl-5,7-dihydro-3H-imidazo[4,5-f]indol-6-one;
3-(5-Isopropyl-7,7-dimethyl-6-oxo-3,5,6,7-tetrahydro-imidazo[4,5-f]indol-2-yl)-1H-indazole-5-carboxylic acid;
3-(5-Ethyl-7,7-dimethyl-6-oxo-3,5,6,7-tetrahydro-imidazo[4,5-f]indol-2-yl)-1H-indazole-5-carboxylic acid amide;
3-(5-Ethyl-7,7-dimethyl-6-oxo-3,5,6,7-tetrahydro-imidazo[4,5-f]indol-2-yl)-1H-indazole-5-carboxylic acid methyl ester;
3-(5-Ethyl-7,7-dimethyl-6-oxo-3,5,6,7-tetrahydro-imidazo[4,5-f]indol-2-yl)-1H-indazole-5-carboxylic acid methoxy-methyl-amide;
2-(5-Amino-1H-indazol-3-yl)-5-ethyl-7,7-dimethyl-5,7-dihydro-3H-imidazo[4,5-f]indol-6-one; and
2-(5-Amino-1H-indazol-3-yl)-5-isopropyl-7,7-dimethyl-5,7-dihydro-1H-imidazo[4,5-f]indol-6-one.

26. The compounds of claim 4, wherein
(a) R¹ is alkyl;
(b) R⁴ and R⁷ each represent hydrogen;
(c) R⁵ is selected from the group consisting of:
(1) alkyl-X, wherein the alkyl group is optionally substituted one or more times with halogen;
(2) heterocyclyl-X—; and
(3) aralkyl-X—, wherein the aryl is a mono- or bicyclic aromatic ring with 6 to 10 ring carbon atoms;
(d) R⁶ is hydrogen; and
(e) X is —NH—, —O— or —C(O)—.

27. A compound of claim 1 selected from the group consisting of:
5-Ethyl-7,7-dimethyl-2-(5-trifluoromethoxy-1H-indazol-3-yl)-5,7-dihydro-3H-imidazo[4,5-f]indol-6-one;
5-Ethyl-7,7-dimethyl-2-[5-(piperidine-1-carbonyl)-1H-indazol-3-yl]-5,7-dihydro-3H-imidazo[4,5-f]indol-6-one;
5-Ethyl-7,7-dimethyl-2-[5-(4-methyl-piperazine-1-carbonyl)-1H-indazol-3-yl]-5,7-dihydro-3H-imidazo[4,5-f]indol-6-one;
5-Ethyl-7,7-dimethyl-2-[5-(morpholine-4-carbonyl)-1H-indazol-3-yl]-5,7-dihydro-3H-imidazo[4,5-f]indol-6-one;
2-[5-(4-Acetyl-piperazine-1-carbonyl)-1H-indazol-3-yl]-5-ethyl-7,7-dimethyl-5,7-dihydro-3H-imidazo[4,5-f]indol-6-one;
5-Ethyl-2-[5-(4-isopropyl-piperazine-1-carbonyl)-1H-indazol-3-yl]-7,7-dimethyl-5,7-dihydro-3H-imidazo[4,5-f]indol-6-one; and
5-Ethyl-7,7-dimethyl-2-[5-(thiomorpholine-4-carbonyl)-1H-indazol-3-yl]-5,7-dihydro-3H-imidazo[4,5-f]indol-6-one.

28. A compound of claim 1 selected from the group consisting of:
5-Ethyl-7,7-dimethyl-2-[5-(thiazolidine-3-carbonyl)-1H-indazol-3-yl]-5,7-dihydro-3H-imidazo[4,5-f]indol-6-one;
5-Ethyl-2-[5-(4-methanesulfonyl-piperazine-1-carbonyl)-1H-indazol-3-yl]-7,7-dimethyl-5,7-dihydro-3H-imidazo[4,5-f]indol-6-one;
2-[5-(1,1 -Dioxo-1λ⁶-thiomorpholine-4-carbonyl)-1H-indazol-3-yl]-5-ethyl-7,7-dimethyl-5,7-dihydro-3H-imidazo[4,5-f]indol-6-one;
5-Ethyl-7,7-dimethyl-2-[5-(1-oxo-1λ⁴-thiomorpholine-4-carbonyl)-1H-indazol-3-yl]-5,7-dihydro-3H-imidazo[4,5-f]indol-6-one;
2-(5-Acetyl-1H-indazol-3-yl)-5-ethyl-7,7-dimethyl-5,7-dihydro-3H-imidazo[4,5-f]indol-6-one;
2-(5-Benzylamino-1H-indazol-3-yl)-5-ethyl-7,7-dimethyl-5,7-dihydro-1H-imidazo[4,5-f]indol-6-one; and
2-(5-Benzyloxy-1H-indazol-3-yl)-5-ethyl-7,7-dimethyl-5,7-dihydro-1H-imidazo[4,5-f]indol-6-one.

29. A compound of claim 4, wherein:
(a) R¹ is hydrogen or alkyl;
(b) R⁴ and R⁷ each represent hydrogen;
(c) R⁵ is selected from the group consisting of:
(1) alkyl-X—;
(2) aryl-X—, wherein the aryl is a mono- or bicyclic aromatic ring with 6 to 10 ring carbon atoms;
(3) arylalkyl-X—, wherein the aryl is a mono- or bicyclic aromatic ring with 6 to 10 ring carbon atoms and wherein the aryl is optionally substituted one or more times with halogen or halogenated (C₁-C₄)alkoxy; and
(4) heteroarylalkyl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) R⁶ is hydrogen; and
(e) X is —NHC(O)—.

30. A compound of claim 1 selected from the group consisting of:
3-(7,7-Dimethyl-6-oxo-3,5,6,7-tetrahydro-imidazo[4,5-f]indol-2-yl)-1H-indazole-5-carboxylic acid ethylamide;
3-(7,7-Dimethyl-6-oxo-3,5,6,7-tetrahydro-imidazo[4,5-f]indol-2-yl)-1H-indazole-5-carboxylic acid benzylamide;
3-(7,7-Dimethyl-6-oxo-3,5,6,7-tetrahydro-imidazo[4,5-f]indol-2-yl)-1H-indazole-5-carboxylic acid phenylamide;
3-(5-Ethyl-7,7-dimethyl-6-oxo-3,5,6,7-tetrahydro-imidazo[4,5-f]indol-2-yl)-1H-indazole-5-carboxylic acid benzylamide;
3-(5-Ethyl-7,7-dimethyl-6-oxo-3,5,6,7-tetrahydro-imidazo[4,5-f]indol-2-yl)-1H-indazole-5-carboxylic acid (pyridin-2-ylmethyl)-amide;
3-(5-Ethyl-7,7-dimethyl-6-oxo-3,5,6,7-tetrahydro-imidazo[4,5-f]indol-2-yl)-1H-indazole-5-carboxylic acid (pyridin-3-ylmethyl)-amide; compound with acetic acid; and
3-(5-Ethyl-7,7-dimethyl-6-oxo-3,5,6,7-tetrahydro-imidazo[4,5-f]indol-2-yl)-1H-indazole-5-carboxylic acid (pyridin-4-ylmethyl)-amide.

31. A compound of claim 1 selected from the group consisting of:
3-(5-Ethyl-7,7-dimethyl-6-oxo-3,5,6,7-tetrahydro-imidazo[4,5-f]indol-2-yl)-1H-indazole-5-carboxylic acid phenylamide;
3-(5-Ethyl-7,7-dimethyl-6-oxo-3,5,6,7-tetrahydro-imidazo[4,5-f]indol-2-yl)-1H-indazole-5-carboxylic acid ethylamide;
3-(5-Ethyl-7,7-dimethyl-6-oxo-3,5,6,7-tetrahydro-imidazo[4,5-f]indol-2-yl)-1H-indazole-5-carboxylic acid 2,4-difluoro-benzylamide;
3-(5-Ethyl-7,7-dimethyl-6-oxo-3,5,6,7-tetrahydro-imidazo[4,5-f]indol-2-yl)-1H-indazole-5-carboxylic acid 3-trifluoromethoxy-benzylamide;
3-(5-Ethyl-7,7-dimethyl-6-oxo-3,5,6,7-tetrahydro-imidazo[4,5-f]indol-2-yl)-1H-indazole-5-carboxylic acid 4-difluoromethoxy-benzylamide;
3-(5-Ethyl-7,7-dimethyl-6-oxo-3,5,6,7-tetrahydro-imidazo[4,5-f]indol-2-yl)-1H-indazole-5-carboxylic acid 3-chloro-benzylamide; and
3-(5-Ethyl-7,7-dimethyl-6-oxo-3,5,6,7-tetrahydro-imidazo[4,5-f]indol-2-yl)-1H-indazole-5-carboxylic acid 4-trifluoromethoxy-benzylamide.

32. The compounds of claim 4, wherein:
(a) R¹ is alkyl;
(b) R⁴ and R⁷ each represent hydrogen;
(c) R⁵ is selected from the group consisting of:
(1) cycloalkyl-X—;
(2) heterocyclyl-X—;
(3) alkyl-X—;
(4) aryl-X—, wherein the aryl is a mono- or bicyclic aromatic ring with 6 to 10 ring carbon atoms and wherein the aryl is optionally substituted one or more times with halogen;
(5) arylalkyl-X—, wherein the aryl is a mono- or bicyclic aromatic ring with 6 to 10 ring carbon atoms and wherein the aryl is optionally substituted one or more times with halogen, (C₁-C₄)alkyl, (C₁-C₄)alkoxy; and
(6) heteroaryl-X—, wherein the heteroaryl is a mono- or bicyclic aromatic ring with 5 to 10 ring atoms, which contains up to 4 heteroatoms selected independently from the group consisting of N, O and S with the remaining ring atoms being carbon atoms;
(d) R⁶ is hydrogen; and
(e) X is —C(O)NH—.

33. A compound of claim 1 selected from the group consisting of,
N-[3-(5-Ethyl-7,7-dimethyl-6-oxo-1,5,6,7-tetrahydro-imidazo[4,5-f]indol-2-yl)-1H-indazol-5-yl]-2-o-tolyl-acetamide;
N-[3-(5-Ethyl-7,7-dimethyl-6-oxo-3,5,6,7-tetrahydro-imidazo[4,5-f]indol-2-yl)-1H-indazol-5-yl]-2-phenyl-acetamide;
N-[3-(5-Ethyl-7,7-dimethyl-6-oxo-1,5,6,7-tetrahydro-imidazo[4,5-f]indol-2-yl)-1H-indazol-5-yl]-isonicotinamide;
Pyridine-2-carboxylic acid [3-(5-ethyl-7,7-dimethyl-6-oxo-1,5,6,7-tetrahydro-imidazo[4,5-f]indol-2-yl)-1H-indazol-5-yl]-amide;
N-[3-(5-Ethyl-7,7-dimethyl-6-oxo-1,5,6,7-tetrahydro-imidazo[4,5-f]indol-2-yl)-1H-indazol-5-yl]-2-p-tolyl-acetamide;
2-(3,5-Dimethoxy-phenyl)-N-[3-(5-ethyl-7,7-dimethyl-6-oxo-1,5,6,7-tetrahydro-imidazo[4,5-f]indol-2-yl)-1H-indazol-5-yl]-acetamide;
N-[3-(5-Ethyl-7,7-dimethyl-6-oxo-1,5,6,7-tetrahydro-imidazo[4,5-f]indol-2-yl)-1H-indazol-5-yl]-4-fluoro-benzamide;
N-[3-(5-Ethyl-7,7-dimethyl-6-oxo-1,5,6,7-tetrahydro-imidazo[4,5-f]indol-2-yl)-1H-indazol-5-yl]-2-(4-fluoro-phenyl)-acetamide;
N-[3-(5-Ethyl-7,7-dimethyl-6-oxo-1,5,6,7-tetrahydro-imidazo[4,5-f]indol-2-yl)-1H-indazol-5-yl]-nicotinamide; and
N-[3-(5-Ethyl-7,7-dimethyl-6-oxo-1,5,6,7-tetrahydro-imidazo[4,5-f]indol-2-yl)-1H-indazol-5-yl]-propionamide.

34. A compound of claim 1 selected from the group consisting of:
Cyclopropanecarboxylic acid [3-(5-ethyl-7,7-dimethyl-6-oxo-1,5,6,7-tetrahydro-imidazo[4,5-f]indol-2-yl)-1H-indazol-5-yl]-amide;
N-[3-(5-Ethyl-7,7-dimethyl-6-oxo-1,5,6,7-tetrahydro-imidazo[4,5-f]indol-2-yl)-1H-indazol-5-yl]-benzamide;
Cyclohexanecarboxylic acid [3-(5-ethyl-7,7-dimethyl-6-oxo-1,5,6,7-tetrahydro-imidazo[4,5-f]indol-2-yl)-1H-indazol-5-yl]-amide;
4-Methyl-piperazine-1-carboxylic acid [3-(5-ethyl-7,7-dimethyl-6-oxo-3,5,6,7-tetrahydro-imidazo[4,5-f]indol-2-yl)-1H-indazol-5-yl]-amide;
Piperidine-1-carboxylic acid [3-(5-ethyl-7,7-dimethyl-6-oxo-1,5,6,7-tetrahydro-imidazo[4,5-f]indol-2-yl)-1H-indazol-5-yl]-amide;
Morpholine-4-carboxylic acid [3-(5-ethyl-7,7-dimethyl-6-oxo-1,5,6,7-tetrahydro-imidazo[4,5-f]indol-2-yl)-1H-indazol-5-yl]-amide;
Pyrrolidine-1-carboxylic acid [3-(5-ethyl-7,7-dimethyl-6-oxo-1,5,6,7-tetrahydro-imidazo[4,5-f]indol-2-yl)-1H-indazol-5-yl]-amide;
4-Methyl-piperazine-1-carboxylic acid [3-(5-ethyl-7,7-dimethyl-6-oxo-1,5,6,7-tetrahydro-imidazo[4,5-f]indol-2-yl)-1H-indazol-5-yl]-amide;
N-[3-(5-Ethyl-7,7-dimethyl-6-oxo-3,5,6,7-tetrahydro-imidazo[4,5-f]indol-2-yl)-1H-indazol-5-yl]-acetamide; and
4-Acetyl-piperazine-1-carboxylic acid [3-(5-ethyl-7,7-dimethyl-6-oxo-1,5,6,7-tetrahydro-imidazo[4,5-f]indol-2-yl)-1H-indazol-5-yl]-amide.

35. The compounds of claim 4, wherein:
(a) R¹ is alkyl;
(b) R⁴ and R⁷ each represent hydrogen;
(c) R⁵ is aryl-X—, wherein the aryl is a mono- or bicyclic aromatic ring with 6 to 10 ring carbon atoms and wherein the aryl is optionally substituted one or more times with halogen, nitro, (C₁-C₄)alkyl, (C₁-C₄)alkoxy, halogenated (C₁-C₄)alkoxy or alkylsulfonyl;
(d) R⁶is hydrogen; and
(e) X is —S(O)₂NH—.

36. A compound of claim 1 selected from the group consisting of:
N-[3-(5-Ethyl-7,7-dimethyl-6-oxo-1,5,6,7-tetrahydro-imidazo[4,5-f]indol-2-yl)-1H-indazol-5-yl]-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-4-methoxy-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-2-nitro-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-3-methoxy-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-2-trifluoromethoxy-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-4-fluoro-benzenesulfonamide; and
3-Chloro-N-[3-(5-ethyl-7,7-dimethyl-6-oxo-1,5,6,7-tetrahydro-imidazo[4,5-f]indol-2-yl)-1H-indazol-5-yl]-benzenesulfonamide.

37. A compound of claim 1 selected from the group consisting of:
N-[3-(5-Ethyl-7,7-dimethyl-6-oxo-1,5,6,7-tetrahydro-imidazo[4,5-f]indol-2-yl)-1H-indazol-5-yl]-3-methyl-benzenesulfonamide;
N-[3-(5-Ethyl-7,7-dimethyl-6-oxo-1,5,6,7-tetrahydro-imidazo[4,5-f]indol-2-yl)-1H-indazol-5-yl]-2-methanesulfonyl-benzenesulfonamide;
N-[3-(5-Ethyl-7,7-dimethyl-6-oxo-3,5,6,7-tetrahydro-imidazo[4,5-f]indol-2-yl)-1H-indazol-5-yl]-2,5-difluoro-benzenesulfonamide;
4-Fluoro-N-[3-(5-isopropyl-7,7-dimethyl-6-oxo-1,5,6,7-tetrahydro-imidazo[4,5-f]indol-2-yl)-1H-indazol-5-yl]-benzenesulfonamide;
N-[3-(5-Isopropyl-7,7-dimethyl-6-oxo-1,5,6,7-tetrahydro-imidazo[4,5-f]indol-2-yl)-1H-indazol-5-yl]-2-methanesulfonyl-benzenesulfonamide; and
N-[3-(5-Isopropyl-7,7-dimethyl-6-oxo-1,5,6,7-tetrahydro-imidazo[4,5-f]indol-2-yl)-1H-indazol-5-yl]-2-nitro-benzenesulfonamide.

38. The compounds of claim 4, wherein:
(a) R¹ is alkyl;
(b) R⁴ and R⁷ each represent hydrogen;
(c) R⁵ is arylalkyl-X—, wherein the aryl is a mono- or bicyclic aromatic ring with 6 to 10 ring carbon;
(d) R⁶ is hydrogen; and
(e) X is —OC(O)NH—.

39. A compound of claim 1 which is [3-(5-Ethyl-7,7-dimethyl-6-oxo-1,5,6,7-tetrahydro-imidazo[4,5-f]indol-2-yl)-1H-indazol-5-yl]-carbamic acid benzyl ester.

40. The compounds of claim 4, wherein:
(a) R¹ is alkyl;
(b) R⁴ and R⁷ each represent hydrogen;
(c) R⁵is hydrogen;
(d) R⁶ is selected from the group consisting of:
(1) halogen;
(2) carboxylic acid;
(3) H₂N—C(O)—;
(4) alkyl-X—;
(5) aryl-X—, wherein the aryl is a mono- or bicyclic aromatic ring with 6 to 10 ring carbon atoms; and
(6) arylalkyl-X—, wherein the aryl is a mono- or bicyclic aromatic ring with 6 to 10 ring carbon atoms; and
(e) X is —NHC(O)—.

41. A compound of claim 1 selected from the group consisting of:
2-(6-Bromo-1H-indazol-3-yl)-5-ethyl-7,7-dimethyl-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-1H-indazole-6-carboxylic acid;
3-(7,7-Dimethyl-6-oxo-3,5,6,7-tetrahydro-imidazo[4,5-f]indol-2-yl)-1H-indazole-6-carboxylic acid benzylamide;
3-(7,7-Dimethyl-6-oxo-3,5,6,7-tetrahydro-imidazo[4,5-f]indol-2-yl)-1H-indazole-6-carboxylic acid ethylamide;
3-(7,7-Dimethyl-6-oxo-3,5,6,7-tetrahydro-imidazo[4,5-f]indol-2-yl)-1H-indazole-6-carboxylic acid phenylamide;
3-(5-Ethyl-7,7-dimethyl-6-oxo-3,5,6,7-tetrahydro-imidazo[4,5-f]indol-2-yl)-1H-indazole-6-carboxylic acid ethylamide;
3-(5-Ethyl-7,7-dimethyl-6-oxo-3,5,6,7-tetrahydro-imidazo[4,5-f]indol-2-yl)-1H-indazole-6-carboxylic acid phenylamide;
3-(5-Ethyl-7,7-dimethyl-6-oxo-3,5,6,7-tetrahydro-imidazo[4,5-f]indol-2-yl)-1H-indazole-6-carboxylic acid benzylamide; and
3-(5-Ethyl-7,7-dimethyl-6-oxo-3,5,6,7-tetrahydro-imidazo[4,5-f]indol-2-yl)-1H-indazole-6-carboxylic acid amide.

42. The compounds of claims 1 wherein A is —CH₂—.

43. The compounds of claim 42, wherein:
(a) R¹ is hydrogen or alkyl; and
(b) R⁴, R⁵, R⁶ and R⁷ each represent hydrogen.

44. A compound of claim 1 selected from the group consisting of:
2-(1H-Indazol-3-yl)-8,8-dimethyl-1,5,7,8-tetrahydro-imidazo[4,5-g]quinolin-6-one; and
5-Ethyl-2-(1H-indazol-3-yl)-8,8-dimethyl-3,5,7,8-tetrahydro-imidazo[4,5-g]quinolin-6-one.

45. The compounds of claim 1 wherein R¹ is alkyl.

46. The compounds of claim 1, wherein R⁴, R⁵, R⁶ and R⁷ each represent hydrogen.

47. A process for the preparation of the compounds of formula I, wherein:
a compound of formula II 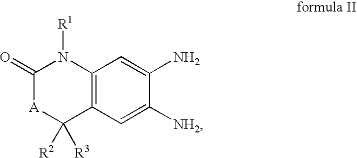
wherein R¹ to R³ and A are defined according to claim 1;
is reacted with-a compound of formula III, 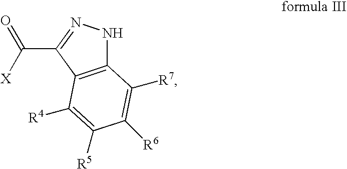
wherein X is —OH, —Cl, —H or —OMe and R⁴ to R⁷ are defined according to claim 1;
to obtain the compound of formula I, 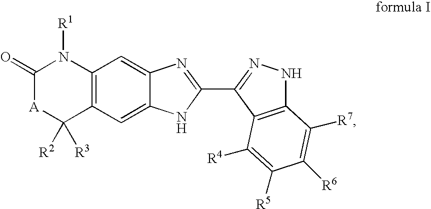
wherein R¹ to R⁷ and A are defined according to claim 1.

48. The process of claim 47 further comprising the step of isolating the compound of formula I from the reaction mixture.

49. The process of claim 47 further comprising the step of converting the compound of formula I into a pharmaceutically acceptable salt or ester.

50. A pharmaceutical composition comprising a therapeutically effective amount of a compound of claim 1 and a pharmaceutically acceptable carrier.

51. A method of preventing or treating a disease or condition mediated by an inappropriate activation of Aurora family tyrosine kinases comprising administering to a person in need thereof a therapeutically effective amount of a compound of claim 1.

52. The method of claim 51 wherein the disease or condition is colorectal cancer, breast cancer, lung cancer, prostate cancer, pancreatic cancer, gastric cancer, bladder cancer, ovarian cancer, melanoma, neuroblastoma, cervical cancer, renal cancer, leukemia or lymphoma.

53. The method of claim 51 wherein the disease or condition is acute-myelogenous leukemia acute lymphocytic leukemia, or gastrointestinal stromal tumor.
